(19) United States
(12) Patent Application Publication
(10) Pub. No.: US 2025/0241902 A1
(43) Pub. Date: Jul. 31, 2025

(54) METHODS, COMPOSITIONS AND SYSTEMS FOR EVALUATION OF VISUAL FUNCTION

(71) Applicant: BELITE BIO, INC, San Diego, CA (US)

(72) Inventors: Tom Yu-Hsin LIN, Taipei City (TW); Webber LIAO, Taipei City (TW)

(21) Appl. No.: 18/856,221

(22) PCT Filed: Apr. 11, 2023
(86) PCT No.: PCT/US2023/018133

§ 371 Date: Oct. 11, 2024

Related U.S. Application Data
(60) Provisional application No. 63/331,189, filed on Apr. 14, 2022.

(51) Int. Cl. A61K 31/454; A61P 27/02; G16H 50/50

(52) U.S. Cl. CPC A61K 31/454; A61P 27/02; G16H 50/50

(57) ABSTRACT

Provided herein is a method of treating a subject with an ocular disease. The method comprises administering to the subject a treatment. The method further comprises determining whether the treatment is therapeutically effective to treat the subject based on a composite biomarker. The composite biomarker is calculated based on at least two variables. At least one of the at least two variables relates to one or more selected from the group comprising vision, visual function, ocular anatomy, ocular physiology, ocular pathology, and a change thereof.

### CROSS REFERENCE TO RELATED APPLICATIONS

This application claims the benefit of U.S. Application No. 63/331,189, filed Apr. 14, 2022, which is hereby incorporated by reference in its entirety.

### BACKGROUND

A need exists for effective clinical evaluation of disease progression and treatment response of ocular diseases and disorders, such as Stargardt disease (STGD) and Age-Related Macular Degeneration (AMD).

### BRIEF SUMMARY OF THE INVENTION

Disclosed herein are methods and systems of treating a subject with an ocular disease. Disclosed herein are methods of treating a subject with an ocular disease, the method comprising: (a) administering to the subject a treatment; (b) determining whether the treatment is therapeutically effective to treat the subject based on a composite biomarker, wherein the composite biomarker is calculated based on at least two variables, wherein at least one of the at least two variables relates to one or more selected from the group comprising vision, visual function, ocular anatomy, ocular physiology, ocular pathology, and a change thereof.

Disclosed herein are methods of treating a subject with an ocular disease, the method comprising: (a) administering to the subject a treatment; (b) performing an assay on the subject to obtain at least two variables, wherein at least one of the at least two variables relates to one or more selected from the group comprising vision, visual function, ocular anatomy, ocular physiology, ocular pathology, and a change thereof, (c) calculating the at least two variables to yield a composite biomarker; and (d) determining whether the treatment is therapeutically effective to treat the subject based on the composite biomarker.

Disclosed herein are methods of treating a subject with an ocular disease, the method comprising: (a) administering to the subject a treatment; (b) performing a first assay on the subject to obtain a first set of at least two variables at a first time point, wherein at least one of the at least two variables relates to one or more selected from the group comprising vision, visual function, ocular anatomy, ocular physiology, ocular pathology, and a change thereof, (c) calculating the first set of the at least two variables to yield a first composite biomarker; (d) performing a second assay on the subject to obtain a second set of the at least two variables at a second time point; (e) calculating the second set of the at least two variables to yield a second composite biomarker; and (f) determining whether the treatment is therapeutically effective to treat the subject based on the first and second composite biomarkers.

Disclosed herein are methods of treating a subject with an ocular disease, the method comprising: (a) administering to the subject a treatment; (b) performing an assay on the subject to obtain at least two variables, wherein at least one of the at least two variables relates to one or more selected from the group comprising vision, visual function, ocular anatomy, ocular physiology, ocular pathology, and a change thereof, and (c) calculating the at least two variables to yield a composite biomarker, wherein the treatment is therapeutically effective to treat the subject when the composite biomarker exceeds a threshold value, wherein the treatment is not therapeutically effective to treat the subject when the composite biomarker does not exceed the threshold value.

Disclosed herein are methods of treating a subject with an ocular disease, the method comprising: (a) administering to the subject a first dosage of a treatment; (b) performing an assay on the subject to obtain at least two variables, wherein at least one of the at least two variables relates to one or more selected from the group comprising vision, visual function, ocular anatomy, ocular physiology, ocular pathology, and a change thereof, (c) calculating the at least two variables to yield a composite biomarker; (d) determining whether the first dosage of the treatment is therapeutically effective to treat the subject based on the composite biomarker; and (e) administering to the subject a second dosage of the treatment, wherein the second dosage is different than the first dosage when the first dosage is determined to be not therapeutically effective to treat the subject, wherein the second dosage is about the same as the first dosage when the first dosage is determined to be therapeutically effective to treat the subject.

Disclosed herein are methods of treating a subject with an ocular disease, the method comprising: (a) performing a first assay on the subject to obtain a first set of at least two variables, wherein at least one of the at least two variables relates to one or more selected from the group comprising vision, visual function, ocular anatomy, ocular physiology, ocular pathology, and a change thereof; (b) calculating the first set of the at least two variables to yield a first composite biomarker; (c) administering a treatment to the subject; (d) performing a second assay on the subject to obtain a second set of the at least two variables; (e) calculating the second set of the at least two variables to yield a second composite biomarker; and (f) determining whether the treatment is therapeutically effective to treat the subject based on the first and second composite biomarkers.

Disclosed herein are methods of treating a subject with an ocular disease, the method comprising: (a) performing an assay on the subject to obtain at least two variables, wherein at least one of the at least two variables relates to one or more selected from the group comprising vision, visual function, ocular anatomy, ocular physiology, ocular pathology, and a change thereof, (b) calculating the at least two variables to yield a composite biomarker; (c) selecting the subject for a treatment based on the composite biomarker; and (d) administering the treatment to the subject.

Disclosed herein are methods of treating a subject with an ocular disease, the method comprising: (a) performing a first assay on the subject to obtain a first set of at least two variables, wherein at least one of the at least two variables relates to one or more selected from the group comprising vision, visual function, ocular anatomy, ocular physiology, ocular pathology, and a change thereof; (b) calculating the first set of the at least two variables to yield a first composite biomarker; (c) selecting the subject for a treatment based on the first composite biomarker; (d) administering the treatment to the subject; (e) performing a second assay on the subject to obtain a second set of the at least two variables; (f) calculating the second set of the at least two variables to yield a second composite biomarker; and (g) determining whether the treatment is therapeutically effective to treat the subject based on the first and second composite biomarkers.

Disclosed herein are methods of treating a subject with an ocular disease, the method comprising: (a) performing an assay on the subject to obtain at least two variables, wherein at least one of the at least two variables relates to one or more selected from the group comprising vision, visual function, ocular anatomy, ocular physiology, ocular pathology, and a change thereof, (b) calculating the at least two variables to yield a composite biomarker; (c) selecting the subject for the treatment when the composite biomarker exceeds a threshold value, or optionally, not selecting the subject for the treatment when the composite biomarker does not exceed the threshold value; and (d) administering to the subject the treatment when the subject is selected for the treatment, or optionally, not administering to the subject a treatment when the subject is not selected for the treatment.

Disclosed herein are methods of treating a subject with an ocular disease, the method comprising: (a) performing a first assay on the subject to obtain a first set of at least two variables, wherein at least one of the at least two variables relates to one or more selected from the group comprising vision, visual function, ocular anatomy, ocular physiology, ocular pathology, and a change thereof; (b) calculating the first set of the at least two variables to yield a first composite biomarker; (c) selecting the subject for a treatment based on the first composite biomarker; (d) administering to the subject a first dosage of the treatment; (e) performing a second assay on the subject to obtain a second set of the at least two variables; (f) calculating the second set of the at least two variables to yield a second composite biomarker; (g) determining whether the first dosage of the treatment is therapeutically effective to treat the subject based on the first and second composite biomarkers; and (h) administering to the subject a second dosage of the treatment, wherein the second dosage is different than the first dosage when the first dosage is determined to be not therapeutically effective to treat the subject, wherein the second dosage is about the same as the first dosage when the first dosage is determined to be therapeutically effective to treat the subject.

Disclosed herein are methods of treating a subject with an ocular disease, the method comprising: (a) performing a first assay on the subject to obtain a first set of at least two variables, wherein at least one of the at least two variables relates to one or more selected from the group comprising vision, visual function, ocular anatomy, ocular physiology, ocular pathology, and a change thereof; (b) calculating the first set of the at least two variables to yield a first composite biomarker; (d) administering to the subject a first dosage of a treatment; (e) performing a second assay on the subject to obtain a second set of the at least two variables; (f) calculating the second set of the at least two variables to yield a second composite biomarker; (g) determining that the first dosage of the treatment is therapeutically effective to treat the subject when the difference between the first and second composite biomarkers exceeds a threshold value, or optionally, determining that the first dosage of the treatment is not therapeutically effective to treat the subject when the difference between the first and second composite biomarkers does not exceed the threshold value; and (h) administering to the subject a second dosage of the treatment, wherein the second dosage is different than the first dosage when the first dosage is determined to be not therapeutically effective to treat the subject, wherein the second dosage is about the same as the first dosage when the first dosage is determined to be therapeutically effective to treat the subject.

Disclosed herein are methods of monitoring the outcome of a treatment for an ocular disease in a subject, the method comprising determining that a dosage of the treatment administered to the subject is therapeutically effective to treat the subject by calculating at least two variables of the subject to yield a composite biomarker, wherein at least one of the at least two variables relates to one or more selected from the group comprising vision, visual function, ocular anatomy, ocular physiology, ocular pathology, and a change thereof.

Disclosed herein are methods of treating a subject with an ocular disease comprising administering to the subject a therapeutically effective dosage of a treatment, wherein the subject is selected for the treatment based on a composite biomarker, wherein the composite biomarker is calculated based on at least two variables of the subject, wherein at least one of the at least two variables relates to one or more selected from the group comprising vision, visual function, ocular anatomy, ocular physiology, ocular pathology, and a change thereof.

Disclosed herein are methods comprising selecting a subject with an ocular disease for a treatment based on a composite biomarker, wherein the composite biomarker is calculated based on at least two variables of the subject, wherein at least one of the at least two variables relates to one or more selected from the group comprising vision, visual function, ocular anatomy, ocular structure, ocular physiology, ocular pathology, and a change thereof.

Disclosed herein are methods of selecting a subject with an ocular disease for a treatment, the method comprising: (a) calculating at least two variables of the subject to yield a composite biomarker, wherein at least one of the at least two variables relates to one or more selected from the group comprising vision, visual function, ocular anatomy, ocular physiology, ocular pathology, and a change thereof; and (b) selecting the subject for the treatment when the composite biomarker exceeds a threshold value, or optionally, not selecting the subject for the treatment when the composite biomarker does not exceed the threshold value.

Disclosed herein are methods of selecting a subject with an ocular disease for a treatment, the method comprising: (a) performing an assay on the subject to obtain at least two variables, wherein at least one of the at least two variables relates to one or more selected from the group comprising vision, visual function, ocular anatomy, ocular physiology, ocular pathology, and a change thereof; (b) calculating the at least two variables to yield a composite biomarker; and (c) selecting the subject for the treatment when the composite biomarker exceeds a threshold value, or optionally, not selecting the subject for the treatment when the composite biomarker does not exceed the threshold value.

Disclosed herein are methods of predicting a subject's therapeutic response to a treatment for an ocular disease, the method comprising: (a) calculating at least two variables of the subject to yield a composite biomarker, wherein at least one of the at least two variables relates to one or more selected from the group comprising vision, visual function, ocular anatomy, ocular physiology, ocular pathology, and a change thereof, and (b) predicting the subject's therapeutic response to the treatment based on the composite biomarker.

Disclosed herein are methods of predicting a subject's therapeutic response to a treatment for an ocular disease, the method comprising: (a) performing an assay on the subject to obtain at least two variables, wherein at least one of the at least two variables relates to one or more selected from the group comprising vision, visual function, ocular anatomy, ocular physiology, ocular pathology, and a change thereof; (b) calculating the at least two variables to yield a composite biomarker; and (c) determining that the subject is likely to have therapeutic response to the treatment when the composite biomarker exceeds a threshold value, or optionally, determining that the subject is likely not to have therapeutic response to the treatment when the composite biomarker does not exceed the threshold value.

Disclosed herein are methods comprising determining a dosage of a treatment for a subject with an ocular disease based on a composite biomarker, wherein the composite biomarker is calculated based on at least two variables of the subject, wherein at least one of the at least two variables relates to one or more selected from the group comprising vision, visual function, ocular anatomy, ocular physiology, ocular pathology, and a change thereof.

Disclosed herein are methods of determining a dosage of a treatment for a subject with an ocular disease, the method comprising: (a) performing an assay on the subject to obtain at least two variables, wherein at least one of the at least two variables relates to one or more selected from the group comprising vision, visual function, ocular anatomy, ocular physiology, ocular pathology, and a change thereof; (b) calculating the at least two variables to yield the composite biomarker; and (c) determining a dosage of the treatment for the subject based on the composite biomarker.

Disclosed herein are methods of determining a dosage of a treatment for an ocular disease in a subject, the method comprising: (a) performing a first assay on the subject to obtain a first set of at least two variables, wherein at least one of the at least two variables relates to one or more selected from the group comprising vision, visual function, ocular anatomy, ocular physiology, ocular pathology, and a change thereof, (b) calculating the first set of the at least two variables to yield a first composite biomarker; (b) administering the treatment to the subject; (b) performing a second assay on the subject to obtain a second set of the at least two variables; (c) calculating the second set of the at least two variables to yield a second composite biomarker; and (d) determining a dosage of the treatment for the subject based on the first and second composite biomarkers.

Disclosed herein are methods comprising administering to a subject with an ocular disease a second dosage of a treatment, wherein a first dosage of the treatment previously administered to the subject was determined to lack therapeutic efficacy to treat the ocular disease in the subject based on a composite biomarker calculated from at least two variables of the subject, wherein at least one of the at least two variables relates to one or more selected from the group comprising vision, vision, visual function, ocular anatomy, ocular physiology, ocular pathology, and a change thereof, wherein the second dosage is different than the first dosage.

Disclosed herein are methods of determining a dosage of a treatment for an ocular disease in a subject, the method comprising: (a) administering to the subject a dosage of the treatment; (b) performing an assay on the subject to obtain at least two variables, wherein at least one of the at least two variables relates to one or more selected from the group comprising vision, visual function, ocular anatomy, ocular physiology, ocular pathology, and a change thereof, (c) calculating the at least two variables to yield a composite biomarker; (d) determining whether the dosage of the treatment is therapeutically effective to treat the subject with the ocular disease based on the composite biomarker.

Disclosed herein are methods of determining a dosage of a treatment for a subject with an ocular disease, the method comprising: (a) performing a first assay on the subject to obtain a first set of at least two variables, wherein at least one of the at least two variables relates to one or more selected from the group comprising vision, visual function, ocular anatomy, ocular physiology, ocular pathology, and a change thereof, (b) calculating the first set of the at least two variables to yield a first composite biomarker; (c) administering to the subject a dosage of the treatment; (d) performing a second assay on the subject to obtain a second set of the at least two variables; (e) calculating the second set of the at least two variables to yield a second composite biomarker; and (f) determining that the dosage of the treatment is therapeutically effective to treat the subject when the difference between the first and second composite biomarkers exceeds a threshold value, or optionally, determining that the dosage of the treatment is not therapeutically effective to treat the subject when the difference between the first and second composite biomarkers does not exceed the threshold value.

In some embodiments, the ocular disease comprises maculopathy, retinopathy, retina atrophy, macular atrophy, macular degeneration, age-related macular degeneration (AMD), Stargardt's Disease (STGD), RP (Retinitis pigmentosa), an ABCA4 gene mutation, or a combination thereof. In some embodiments, a sample from the subject with the ocular disease has an expression level of retinol binding protein 4 (RBP4) of at least a threshold value. In some embodiments, the threshold value is about 25 μg/ml. In some embodiments, the threshold value is about 35 μg/ml. In some embodiments, the threshold value is about 25 μg/ml to about 100 μg/ml. In some embodiments, the expression level of RBP4 is measured by an assay comprising an antibody assay, an electrophoresis assay, an immunoassay, a radioimmunoassay, a chromatographic assay, a mass spectrometry assay, a microarray-based detection assay, a polymerase chain reaction assay, a sequencing assay, an immunohistochemistry assay, or any combination thereof. In some embodiments, the antibody assay comprises ELISA. In some embodiments, a sample from the subject with the ocular disease has an expression level of vitamin A of at least a threshold value. In some embodiments the presence or absence of one or more genomic variants indicates that the subject has the ocular disease. In some embodiments the presence or absence of one or more genomic variants indicates that the subject has Stargardt disease. In some embodiments, the threshold value is about 150 ng/mL. In some embodiments, wherein the threshold value is about 225 ng/mL or about 390 ng/mL. In some embodiments, the threshold value is about 150 ng/mL to about 500 ng/mL. In some embodiments, the expression level of vitamin is measured by an assay comprising an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. In some embodiments, the sample comprises a blood sample. In some embodiments, the expression level of RBP4 or vitamin A is measured from plasma or serum derived from the blood sample. In some embodiments, the presence or absence of one or more genomic variants selected from the group comprising rs4147863, rs2275029, rs1800739, rs4147857, rs4147856, rs1801555, and rs1801574 indicates that the subject has the ocular disease. In some embodiments, the one or more genomic variants comprises at least four of rs4147863, rs2275029, rs1800739, rs4147857, rs4147856, rs1801555, or rs1801574. In some embodiments, the one or more genomic variants comprises at least five of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555, rs1762114, rs55860151, rs1800549, rs3112831, rs4147830, rs2297634, or rs4847281.

In some embodiments, the treatment comprises a pharmaceutical composition comprising an RBP4 inhibitor or a compound that is configured to reduce blood RBP4 concentration in the subject. In some embodiments, the pharmaceutical composition comprises a compound having the structure of Formula (I):

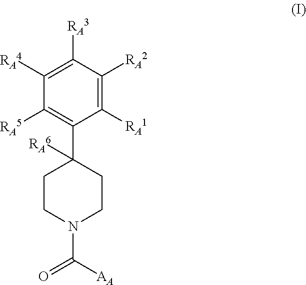

wherein: R_{A}¹, R_{A}², R_{A}³, R_{A}⁴, and R_{A}⁵ are each independently H, halogen, CF₃, or C₁-C₄ alkyl, wherein two or more of R_{A}¹, R_{A}², R_{A}³, R_{A}⁴, and R_{A}⁵ are other than H; R_{A}⁶ is H, OH, or halogen; and AA has the structure:

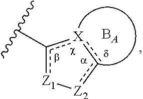

wherein α, β, χ, and δ are each independently absent or present, and when present each is a bond; X is C or N; Z₁ is N; Z₂ is N or NR_{A}⁹, wherein R_{A}⁹ is H, C₁-C₄ alkyl, or oxetane; B_{A} is a substituted or unsubstituted 5, 6, or 7 membered ring structure; or a pharmaceutically acceptable salt thereof.

In some embodiments, the compound has the structure

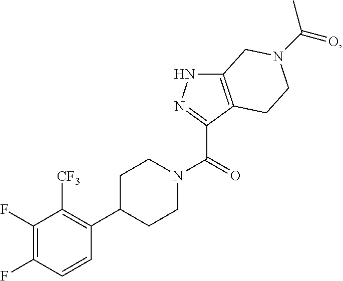 [SMILES: CC(=O)N1CCc2c(C(=O)N3CCC(c4ccc(F)c(F)c4C(F)(F)F)CC3)n[nH]c2C1]

or a pharmaceutically acceptable salt thereof. In some embodiments, the compound has the structure

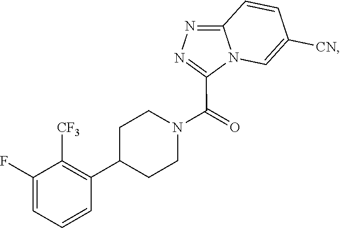 [SMILES: N#Cc1ccc2nnc(C(=O)N3CCC(c4cccc(F)c4C(F)(F)F)CC3)n2c1]

or a pharmaceutically acceptable salt thereof.

In some embodiments, the pharmaceutical composition comprises a compound having the structure of Formula (II):

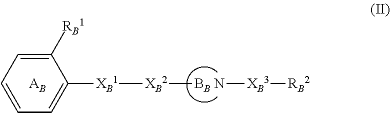

wherein: ring A_{B} is benzene optionally further substituted; R_{B}¹ is an optionally substituted branched C₃-C₆ alkyl group; X_{B}¹ is O, S, SO, SO₂, or NH; X_{B}² is a bond or a C₁-C₃ alkylene group; ring B_{B} is azetidine or piperidine; X_{B}³ is CO or SO₂; R_{B}² is an optionally substituted hydrocarbon group, an optionally substituted heterocyclic group, an optionally substituted hydroxy group, an optionally substituted mercapto group, a cyano group, a nitro group, an acyl group, or a halogen atom; or a pharmaceutically acceptable salt thereof.

In some embodiments, the compound is 4-(3-(2-tert-butylphenoxy)azetidin-1-yl)-4-oxobutanoic acid, 3-{3-[(2-tert-butyl-4-fluorophenoxy)methyl]azetidin-1-yl}-3-oxopropanoic acid, 2-{[3-(2-tert-butyl-4-chlorophenoxy)azetidin-1-yl]carbonyl}pyridine, 4-[3-(2-tert-butyl-4-chlorophenoxy)azetidin-1-yl]-4-oxobutanoic acid, {3-[(2-tert-butyl-4-chlorophenoxy)methyl]azetidin-1-yl}(oxo)acetic acid, {3-[(2-tert-butylphenoxy)methyl]azetidin-1-yl}(oxo)acetic acid, 3-{3-[(2-tert-butylphenoxy)methyl]azetidin-1-yl}-3-oxopropanoic acid, {4-[(2-tert-butyl-4-chlorophenoxy)methyl]piperidin-1-yl}(oxo)acetic acid, [4-(2-tert-butylphenoxy)piperidin-1-yl](oxo)acetic acid, or {4-[(2-tert-butylphenoxy)methyl]piperidin-1-yl}(oxo)acetic acid, or a pharmaceutically acceptable salt thereof.

In some embodiments, the pharmaceutical composition comprises a compound having the structure of Formula (III):

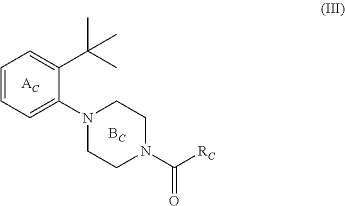

wherein: ring A_{C} is a benzene ring optionally substituted by 1 to 3 substituents selected from the group consisting of (a) a halogen atom, and (b) a C₁-C₆ alkyl group; ring B_{C} is a piperazine ring optionally substituted by 1 to 3 substituents selected from the group consisting of (a) a halogen atom, (b) a C₁-C₆ alkyl group optionally substituted by 1 to 3 halogen atoms, and (c) a C₁-C₆ alkoxy group optionally substituted by 1 to 3 halogen atoms; and R_{C} is (1) an optionally substituted C₁-C₁₀ alkyl group, (2) an optionally substituted C₆-C₁₄ aryl group, (3) an optionally substituted 5- or 6-membered aromatic heterocyclic group, (4) an optionally substituted amino group, (5) an optionally substituted carboxy group, or (6) an optionally substituted carbamoyl group, or a pharmaceutically acceptable salt thereof.

In some embodiments, the compound is N-{[4-(2-tert-butylphenyl)piperazin-1-yl]carbonyl}glycine, 3-[4-(2-tert-butylphenyl)piperazin-1-yl]-3-oxopropanoic acid, [4-(2-tert-butyl-4-chlorophenyl)piperazin-1-yl](oxo)acetic acid, 5-{2-[4-(2-tert-butylphenyl)piperazin-1-yl]-2-oxoethyl}imidazolidine-2,4-dione, [(5-{[4-(2-tert-butylphenyl)piperazin-1-yl]carbonyl}isoxazol-3-yl)oxy]acetic acid, or a pharmaceutically acceptable salt thereof.

In some embodiments, the pharmaceutical composition comprises a compound having the structure of Formula (IV):

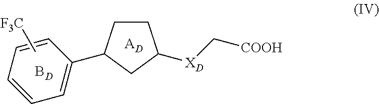

wherein: ring A_{D} is a 5-membered non-aromatic heterocycle optionally further substituted by one oxo group; ring B_{D} is a benzene ring optionally further substituted by 1 to 4 substituents; and X_{D} is 0, CH₂O, OCH₂, CH₂, (CH₂)₂, S, CH₂S, SCH₂, S(O), CH₂S(O), S(O)CH₂, S(O)₂, CH₂S(O)₂, or S(O)₂CH₂; or a pharmaceutically acceptable salt thereof.

In some embodiments, the compound is ({(3S)-1-[3,5-bis(trifluoromethyl)phenyl]pyrrolidin-3-yl}oxy)acetic, ({1-[4-chloro-3-(trifluoromethyl)phenyl]pyrrolidin-3-yl}sulfanyl)acetic acid, 3-{(2R,5S)-5-[3,5-bis(trifluoromethyl)phenyl]tetrahydrofuran-2-yl}propanoic acid, or a pharmaceutically acceptable salt thereof.

In some embodiments, the pharmaceutical composition comprises a compound having the structure of Formula (V):

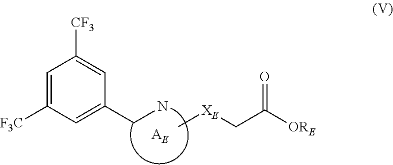

wherein, ring A_{E} is a pyrazole ring, a pyridine ring, an oxazole ring, an imidazole ring, or a pyrimidine ring; X_{E} is S, optionally substituted alkylene, or O; and R_{E} is a hydrogen atom or a C₁-C₆ alkyl group; or a pharmaceutically acceptable salt thereof.

In some embodiments, the compound is ((4-(3,5-bis(trifluoromethyl)phenyl)-1,3-oxazol-2-yl)sulfanyl)acetic acid, ethyl ((6-(3,5-bis(trifluoromethyl)phenyl)-pyridin-3-yl)sulfanyl)acetic acid, ((6-(3,5-bis(trifluoromethyl)-phenyl)pyridine-3-yl)sulfanyl)acetic acid, or 3-(3-(3,5-bis(trifluoromethyl)phenyl)-1H-pyrazol-1-yl)butanoic acid, or 3-{3-[3,5-bis(trifluoromethyl)phenyl]-1H-pyrazol-1-yl}propanoic acid (also known as STG-001).

In some embodiments, the at least one of the at least two variables relates to one or more selected from the group comprising visual acuity, average score of functional visual questionnaire, and a measurement obtained from an imaging technology. In some embodiments, the at least one of the at least two variables relates to one or more selected from the group comprising a change in visual acuity, a change in average score of functional visual questionnaire, and a change in a measurement obtained from an imaging technology for detecting or quantifying a change in ocular structure or visual function. In some embodiments, the at least one of the at least two variables relates to a change in visual acuity, a change in average score of functional visual questionnaire, and a change in a measurement obtained from an imaging technology for detecting or quantifying a change in optic atrophy or visual function. In some embodiments, the at least two variables relate to a change in visual acuity, a change in average score of functional visual questionnaire, and a change in a measurement obtained from an imaging technology for detecting or quantifying a change in optic atrophy or visual function. In some embodiments, the at least two variables relate to a change in visual acuity and a change in average score of functional visual questionnaire. In some embodiments, the at least two variables relate to a change in visual acuity and a change in a measurement obtained from an imaging technology for detecting or quantifying a change in optic atrophy or visual function. In some embodiments, the at least two variables relate to a change in average score of functional visual questionnaire and a change in a measurement obtained from an imaging technology for detecting or quantifying a change in optic atrophy or visual function. In some embodiments, the at least two variables all relate to a change in visual acuity. In some embodiments, the at least two variables all relate to a change in average score of functional visual questionnaire. In some embodiments, the at least two variables all relate to a change in a measurement obtained from an imaging technology for detecting or quantifying a change in optic atrophy or visual function. In some embodiments, the at least one of the at least two variables relates a change in ocular structure, a change in ocular pathology, a change in retinal atrophy, a change in macular atrophy, a change in macular degeneration, or a combination thereof. In some embodiments, two of the at least two variables correlate with each other. In some embodiments, one of the at least two variables correlates with visual acuity. In some embodiments, one of the at least two variables correlates with a change in visual acuity. In some embodiments, all of the at least two variables correlate with a change in visual acuity. In some embodiments, one of the at least two variables correlates with a state of the ocular disease. In some embodiments, one of the at least two variables correlates with progression of the ocular disease. In some embodiments, the at least two variables comprise a value obtained from a logarithm of the minimum angle of resolution (log MAR) chart, a Snellen chart, a best corrected visual acuity (BCVA) test, an early treatment diabetic retinopathy study (ETDRS) letter test, or a combination thereof. In some embodiments, the at least two variables comprise a value obtained from a functional visual questionnaire best comprising one or more questions related to the following: (a) the subject's confidence of moving to different places in the daytime or nighttime; (b) the subject's ability to recognize people, to take part in sport or to get information; and (c) the subject's ability for social interaction or work efficiency, wherein each of the one or more question s is scored with respect to the subject. In some embodiments, the each of the one or more questions is scored in a severity scale of 0-4 with respect to the subject according to the following: (a) an answer of “NEVER” is scored 0; (b) an answer of “ALMOST NEVER” is scored 1; (c) an answer of “SOMETIMES” is scored 2; (d) an answer of “ALMOST ALWAYS” is scored 3; and (e) an answer of “ALWAYS” is scored 4. In some embodiments, the at least two variables comprise a value obtained from microperimetry (MP), perimetry, or both. In some embodiments, the MP comprises measuring a spatial map of retinal sensitivity of the subject. In some embodiments, the MP comprises measuring a mean retina sensitivity of the subject, or a change thereof. In some embodiments, the at least two variables comprise a value obtained from one or more selected from the group comprising fundus photography, fundus angiography (FA), fundus autofluorescence (FAF), spectral domain-optical coherence tomography (SD-OCT), quantitative Autofluorescence (qAF), infrared (IR) imaging, optical coherence tomography-angiography (OCT-A), and widefield imaging. In some embodiments, the at least two variables comprise one or more selected from the group comprising an area of questionable decreased autofluorescence (QDAF), an area of definite decreased autofluorescence (DDAF), an area of decreased autofluorescence (DAF), a retina thickness, an ellipsoid zone (EZ) defect width, a central subfield retina thickness (CST), an outer subfield retina thickness (OST), a middle subfield retina thickness (MST), and a change thereof. In some embodiments, the area of decreased autofluorescence (DAF) comprises the area of questionable decreased autofluorescence (QDAF) and the area of definite decreased autofluorescence (DDAF). In some embodiments, the at least two variables comprise two or more selected from the group comprising visual acuity, QDAF, DAF, EZ defect width, or a change thereof. In some embodiments, the at least two variables comprise visual acuity, QDAF, DAF, EZ defect width, or a change thereof. In some embodiments, the EZ defect width correlates with the visual acuity. In some embodiments, the EZ defect width correlates with the QDAF. In some embodiments, the EZ defect width correlates with the DAF. In some embodiments, the visual acuity correlates with the QDAF. In some embodiments, the EZ defect width correlates with the visual acuity, QDAF, and DAF. In some embodiments, the visual acuity correlates with the EZ defect width and QDAF. In some embodiments, the EZ defect width correlates with the visual acuity, QDAF, and DAF with a correlation coefficient of more than about 0.8 and a p value of less than about 0.05. In some embodiments, the visual acuity correlates with the EZ defect width and QDAF with a p value of less than about 0.05. In some embodiments, the at least two variables of the subject are obtained from both the right and left eyes of the subject. In some embodiments the method further comprises predicting whether prognostic potentials of treatment improve based on the one more composite biomarkers. In some embodiments the treatment is therapeutically effective to treat the subject when the composite biomarker exceeds the threshold value. In some embodiments the treatment is not therapeutically effective to treat the subject when the composite biomarker does not exceed the threshold value.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A and FIG. 1B illustrate a decrease in autofluorescence (AF) using qAF in the right (FIG. 1A) and left (FIG. 1B) eyes of subject 3 after treatment with RBP4 inhibitor. The left image in each figure is labeled Phase 2 Baseline, and the right image in each figure is labeled Phase 2 Month 6. qDAF values are labeled for each image (left to right): 335, 275, 351, and 268.

FIG. 1C illustrates fundus autofluorescence (FAF) of the right eye of subject 3 at phase 2 baseline (left) and phase 2 month 6 (right). Lesion areas are labeled 2.7 mm² (left) and 2.7 mm² (right).

FIG. 1D illustrates an EZ defect analysis of subject 3's left eye at Phase 2 Baseline (left) and Phase 2 Month 6 (right). The red lines are labeled 2.91 mm (left) and 2.71 mm (right).

FIG. 1E illustrates changes of MST and OST (retinal thickness) in the left eye of subject 3 after treatment with RBP4 inhibitor. The top images are labeled Phase 2 Baseline, and the bottom images are labeled Phase 2 Month 6. The left side of each image shows regions of the eye and average thickness. For phase 2 baseline, the average thickness values in the legend are (top to bottom): marker (97 micron); center (97 micron); central min (97 micron); and central max (202 micron). For phase 2 month 6, the average thickness values in the legend are (top to bottom): marker (112 micron); center (112 micron); central min (104 micron); and central max (216 micron). The right side of each image shows regions of the eye and retinal thickness; the retinal thickness scales on the right are labeled 0 to 800 at 100 unit interval; the size scale inset in the image is labeled 200 micron.

FIG. 2A illustrates stabilization of QDAF in size in the left eye of subject 4 after treatment with RBP4 inhibitor. The left image is labeled Phase 2 Baseline, and the right image is labeled Phase 2 Month 6.

FIG. 2B illustrates changes of MST and OST (retinal thickness) in the left eye of subject 4 after treatment with RBP4 inhibitor. The top images are labeled Phase 2 Baseline, and the bottom images are labeled Phase 2 Month 6. The left side of each image shows regions of the eye and average thickness. For phase 2 baseline, the average thickness values in the legend are (top to bottom): marker (60 micron); center (63 micron); central min (57 micron); and central max (218 micron). For phase 2 month 6, the average thickness values in the legend are (top to bottom): marker (56 micron); center (58 micron); central min (54 micron); and central max (199 micron). The right side of each image shows regions of the eye and retinal thickness; the retinal thickness scales on the right are labeled 0 to 800 at 100 unit interval; the size scale inset in the image is labeled 200 micron.

FIG. 2C illustrates an EZ defect analysis of subject 4's right eye at Phase 2 Baseline (left) and Phase 2 Month 6 (right). The red lines are labeled 1.79 mm (left) and 1.85 mm (right).

FIG. 3A illustrates changes of SD-OCT in the right eye of subject 11 after treatment with RBP4 inhibitor. The left image is labeled Phase 2 Baseline, and the right image is labeled Phase 2 Month 6.

FIG. 3B illustrates the change of autofluorescence in the right eye of subject 11 after treatment with RBP4 inhibitor. The left image is labeled Phase 2 Baseline, and the right image is labeled Phase 2 Month 6.

FIG. 3C illustrates an EZ defect analysis of subject 11's left eye at Phase 2 Baseline (left) and Phase 2 Month 6 (right). The red lines are labeled 2.20 mm (left) and 2.29 mm (right).

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Ocular Disease and Treatment

Provided herein are methods for evaluation of disease progression and disease treatment of an ocular disease in a subject. The ocular disease comprises a disease that affects visual function, ocular structure, ocular anatomy, retina structure, retina function, retina pathology, macular structure, macular function, macular pathology, or a combination thereof. For example, the ocular disease comprises, but not limited to, maculopathy, retinopathy, retina atrophy, macular atrophy, macular degeneration, diabetic retinopathy, an inherited retina disease, age-related macular degeneration (AMD), Stargardt's Disease (STGD), RP (Retinitis pigmentosa), an ABCA4 gene mutation, or a combination thereof. Patients with retina degenerative diseases may experience loss or decline of visual acuity (VA) or pathological or structural changes in retina. Age-Related Macular Degeneration (AMD) is a common eye condition and a leading cause of vision loss among people age 50 and older. It causes damage to the macula, a small spot near the center of the retina and the part of the eye needed for sharp, central vision. As AMD progresses, a blurred area near the center of vision is a common symptom. Over time, the blurred area may grow larger and the subject may develop blank spots in his or her central vision. Stargardt's Disease (STGD) is a juvenile maculopathy that shares similar pathology as AMD. STGD is the more prevalent form of inherited macular degeneration and STGD1 is the most common subtype. STGD affects 1 in 10,000 people around the world. Early onset Stargardt disease (<20 years old) is usually associated with worse severity. In retinal degenerative diseases including STGD and AMD, accumulation of lipofuscin which is associated with tissue autofluorescence (AF) in the retinal pigmented epithelium (RPE) has been observed and bisretinoid N-retinyl-N-retinylidene ethanolamine (A2E) is a component of lipofuscin. Accumulation of A2E is likely a cause of retinal atrophy. Excessive accumulation of lipofuscin has been recognized as a common pathogenetic pathway in various retinal diseases, including AMD, that precedes photoreceptor degeneration. Appearance of AF correlates with disease pathology. Quantitative AF may be a direct assessment of the relative level of A2E and related bisretinoids in retinal tissue. It has been shown that there were significantly elevated AF levels in childhood-onset STGD 1 compared to adult-onset STGD 1. AF in childhood-onset STGD 1 is up to 8-fold higher than AF in healthy eyes. Childhood-onset STGD 1 has been shown to be characterized by a rapid decline in visual acuity. Adult-onset STGD 1 has been shown to be a milder phenotype with foveal-sparing which allows stable visual acuity for several years. Mutation in the ATP-binding cassette, subfamily A, member 4 (ABCA4) gene may lead to excessive accumulation of the cytotoxic bisretinoid A2E. A2E is the primary source of aberrant autofluorescence (AF) in the retina. The steady increase of AF in STGD 1 and AMD may lead to retinal atrophy and subsequent photoreceptor cell loss which causes a decrease in AF. These retinal lesions are referred to as areas of definitely decreased AF (DDAF) and can be quantified using imaging techniques. See Tanna, P., et al. (2016) British Journal of Ophthalmology, 101(1):25-30; Invest Ophthalmol Vis Sci. 2011; 52:9379-9390; Invest Ophthalmol Vis Sci. 2015; 56:8179-8186; Invest Ophthalmol Vis Sci. 2014; 55:2841-2852; Ophthalmology 2015; 122:335-344; Br J Ophthalmol 2017; 101:25-30; AM J Ophthalmol 2020; 211:159-175; JAMA Ophthalmol 2017; 135:1232-1241; Invest Ophthalmol Vis Sci. 2016; 57:5186-5191. Retinol-binding protein 4 (RBP4), the retinol transporter in blood, is secreted from adipocytes and the liver. Lowering levels of RBP4 can lead to reduction in the accumulation of lipofuscin and the accumulation of lipofuscin results in vision loss in diseases like AMD, dry (atrophic) AMD, and STGD. Lowering RBP4 may reduce the accumulation of lipofuscin in the retina and thus delay or stop vision loss in AMD or STGD resulting from excessive accumulation of lipofuscin in the retina. RBP4 inhibitors disclosed herein may inhibit RBP4 to slow down bisretinoids/A2E accumulation.

### Evaluation of Visual Function

There are a number of approaches for evaluating ocular disease or visual function for various purposes including, but not limited to, selection of a subject for a treatment, assessment of disease progression in a subject, assessment of potential therapeutic effect of a treatment on a subject, assessment of a subject's therapeutic or clinical response to a treatment, determination of a dosage of a treatment for a subject, or a combination thereof. These approaches comprises, but not limited to, retinal imaging methods, imaging modalities and functional tests. Imaging modalities usually use specialized cameras with/without non-invasive laser light or fluorescent dye. Imaging modalities comprises, but not limited to, fundus photography, fundus angiography (FA), fundus autofluorescence (FAF), spectral domain-optical coherence tomography (SD-OCT), quantitative autofluorescence (qAF), infrared (IR) imaging, optical coherence tomography-angiography (OCT-A), and widefield imaging. Functional tests usually evaluate visual function or impact on daily activities or behavior. Functional tests comprises test of visual acuity, functional visual questionnaire, microperimetry, perimetry, and other behavioral function tests. These evaluation approaches is used to measure a number of variables related to vision, visual function, visual structure, ocular anatomy, retinal atrophy, retinal degeneration, macular degeneration, retina physiology or pathology, macular physiology or pathology, or a change thereof. For examples, these variables comprises, but not limited to, visual acuity (VA), a functional visual questionnaire score, a logarithm of the minimum angle of resolution (log MAR) chart score, a Snellen chart score, a best corrected visual acuity (BCVA) test score, an early treatment diabetic retinopathy study (ETDRS) letter test score, retinal sensitivity, a size of questionable decreased autofluorescence (QDAF), a size of definite decreased autofluorescence (DDAF), a size of decreased autofluorescence (DAF), a retina thickness, an ellipsoid zone (EZ) defect width, a central subfield retina thickness (CST), an outer subfield retina thickness (OST), a middle subfield retina thickness (MST), or a change thereof, or any other variable related to visual function.

Disclosed herein are methods, compositions and systems for evaluation of visual function. In some embodiments, the methods, compositions or systems disclosed herein may combine at least two variables to generate a composite biomarker. In some embodiments, the composite biomarker is used for evaluation of an ocular disease or visual function. In some embodiments, the composite biomarker is used for selection of a subject with an ocular disease for a treatment, assessment of ocular disease progression in a subject, assessment of potential therapeutic effect of a treatment for a subject with an ocular disease, assessment of a subject's therapeutic response to a treatment for an ocular disease, determination of a dosage of a treatment for a subject, or any other purpose related to treatment or evaluation of an ocular disease, or a combination thereof. In some embodiments, the at least two variables are obtained from at least two evaluation approaches disclosed herein for an ocular disease including, but not limited to, imaging methods, imaging modalities or functional tests. In some embodiments, the at least two variables comprises measurements, images, analyses, test scores, values, or any other form of data that are obtained from the evaluation approaches for ocular disease. In some embodiments, the composite biomarker may be in the form of a score, a predictive score, a predictive model, or a combination thereof. In some embodiments, the composite biomarker comprises a threshold value. In some embodiments, whether the composite biomarker exceeds the threshold value correlates with a certain outcome of a method disclosed herein. In some embodiments, the composite biomarker is used for selection of a subject for a certain clinical treatment. In some embodiments, the composite biomarker is used for assessment of disease progression in a subject. In some embodiments, the composite biomarker is used for assessment of potential therapeutic effect of a certain treatment for a subject before treatment. In some embodiments, the composite biomarker is used for determination of a dosage of a treatment for a subject. In some embodiments, the composite biomarker is used for assessment of therapeutic effect for a subject during the treatment. In some embodiments, the composite biomarker is used for a combination of purposes disclosed herein thereof. In some embodiments, when the composite biomarker exceeds the threshold value, the subject may be selected for a certain clinical treatment. In some embodiments, when the composite biomarker does not exceed the threshold value, the subject may be not selected for a certain clinical treatment. In some embodiments, when the composite biomarker exceeds the threshold value, the subject may be determined to likely have a certain disease state. In some embodiments, when the composite biomarker does not exceed the threshold value, the subject may be determined not likely to have a certain disease state. In some embodiments, when the composite biomarker exceeds the threshold value, the subject may be determined to likely benefit therapeutically from a treatment before being given the treatment. In some embodiments, when the composite biomarker does not exceed the threshold value, the subject may be determined not likely to benefit therapeutically from a treatment before being given the treatment. In some embodiments, when the composite biomarker exceeds the threshold value, the subject may be determined to likely have a certain therapeutic response to a treatment during the course of the treatment. In some embodiments, when the composite biomarker does not exceed the threshold value, the subject may be determined not likely to have a certain therapeutic response to a treatment during the course of the treatment. In some embodiments, a dosage of a treatment for an ocular disease can be calculated based on the composite biomarker.

In some embodiments, at least one of the at least two variables correlates with a state of an ocular disease. In some embodiments, the at least two variables comprises at least one objective variable. In some embodiments, the at least one objective variable correlates with a state of an ocular disease. In some embodiments, the at least one objective variable comprises a change in ocular structure. In some embodiments, the at least one objective variable comprises an ocular pathological change. In some embodiments, at least one of the at least two variables correlates with another of the at least two variables. In some embodiments, each of the at least two variables correlates with the rest of the at least two variables. In some embodiments, at least one of the at least two variables correlates with visual acuity. In some embodiments, visual acuity may be an end point of a clinical evaluation or clinical treatment. In some embodiments, visual acuity may be a subjective measurement. In some embodiments, the at least two variables comprises visual acuity, a visual questionnaire score, a measurement obtained from an imaging technology for evaluation of an ocular disease, a change thereof, or a combination thereof. In some embodiments, the imaging technology is used to detect and/or quantify a change in retinal atrophy. In some embodiments, the at least two variables comprises a change in visual acuity, a change in a visual questionnaire average score, a measurement from an imaging technology used to detect and/or quantify a change in retinal atrophy. In some embodiments, one of the at least two variables are obtained from a subject's right eye, left eye, or both. In some embodiments, the at least two variables are obtained from at least two subjects with different gender, ethnicity, age, or a combination thereof. In some embodiments, the at least two variables are obtained during at least two clinical phases.

There are a number of feature variables for evaluation of an ocular disease including, but not limited to, a change in visual acuity, a change in functional visual questionnaire average score, a change in measurement obtained from an imaging modality used to detect and/or quantify change in retinal or macular structure/anatomy, such as retinal atrophy, macular atrophy, or macular degeneration in AMD and STGD. These feature variables is used to select a patient for treatment, monitor disease progression and/or evaluate treatment effects.

In some embodiments, visual acuity may be evaluated with a logarithm of the minimum angle of resolution chart (log MAR chart). A log MAR chart comprises rows of letters that are used to estimate visual acuity. Zero log MAR may indicate standard vision, positive values may indicate poor vision and negative values may indicate good vision. In some embodiments, the best corrected visual acuity (BCVA) early treatment diabetic retinopathy study (ETDRS) letters score can be calculated with the following formula:

$$Log MAR score = - 0.02 * Letter Score + 1.7$$

In some embodiments, a modifier of −1 may be added to the change in visual acuity for interpretation of visual acuity improvement. In some embodiments, the Snellen chart may also be used to evaluate visual acuity.

In some embodiments, a functional visual questionnaire may be carried out in a number of questions scored in a severity scale. An average score are obtained from one or more functional visual questionnaire. In some embodiments, the score scale of a visual questionnaire may be any range between (or including) two numbers. In some embodiments, the score scale may be 0 to 4. In some embodiments, an answer of “NEVER” to a question is assigned a score according to the score scale. In some embodiments, an answer of “ALMOST NEVER” to a question is assigned a score according to the score scale. In some embodiments, an answer of “SOMETAIES” to a question is assigned a score according to the score scale. In some embodiments, an answer of “ALMOST ALWAYS” to a question is assigned a score according to the score scale. In some embodiments, an answer of “ALWAYS” to a question is assigned a score according to the score scale. In some embodiments, the answers, “NEVER,” “ALMOST NEVER,” “SOMETIMES,” “ALMOST ALWAYS,” and “ALWAYS,” to a question is assigned a score of 0, 1, 2, 3, 4, respectively, according to the 0-4 score scale. In some embodiments, the answers, “NEVER,” “ALMOST NEVER,” “SOMETIMES,” “ALMOST ALWAYS,” and “ALWAYS,” to a question is assigned a score of 4, 3, 2, 1, 0, respectively, according to the 0-4 score scale.

In some embodiments, the functional visual questionnaire comprises any number of questions. In some embodiments, the functional visual questionnaire comprises any number of questions about 1 to about 200, e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or about 200, or any number therebetween. In some embodiments, the functional visual questionnaire comprises about 24 questions.

In some embodiments, a question in the visual questionnaire relate to a visual function. In some embodiments, the visual function comprises a subject's confidence of moving to different places in the daytime. In some embodiments, the visual function comprises a subject's confidence of moving to different places in the nighttime. In some embodiments, the visual function comprises a subject's confidence of moving to different places in the daytime and nighttime. In some embodiments, the visual function comprises a subject's confidence of moving to different places in the daytime or nighttime. In some embodiments, the visual function comprises a subject's ability to recognize people. In some embodiments, the visual function comprises a subject's ability to take part in sports. In some embodiments, the visual function comprises a subject's ability to get information. In some embodiments, the visual function comprises a subject's ability to recognize people, take part in sports, or get information. In some embodiments, the visual function comprises a subject's social interaction. In some embodiments, the visual function comprises a subject's work efficiency. In some embodiments, the visual function comprises a subject's social interaction and work efficiency. In some embodiments, the visual function comprises a subject's social interaction or work efficiency.

Fundus autofluorescence (FAF) imaging is used as an endpoint in clinical trials. FAF is used to detect lesion growth with more than one timepoint of measurements. The change in lesion area measured by FAF can be plotted against time to calculate lesion growth rate, e.g., how fast or slow a patient's macula or retina is degenerating as compared to placebo. FAF imaging is used to compare the rate of anatomical or structural deterioration or change to evaluate a drug or treatment's therapeutic effect, e.g., reduced worsening of the retina degeneration.

In some embodiments, fundus autofluorescence (FAF) is used for evaluation of ocular disease or visual function. In some embodiments, one or more retina images of FAF is used for detection of atrophic areas. In some embodiments, one or more retina images of FAF is used to gain detailed insight into the health of the retinal pigment epithelium (RPE). In some embodiments, one or more images of FAF may be high-contrast retina images. In some embodiments, two types of decreased autofluorescence (DAF) may be quantified. In some embodiments, one type of DAF may be definite DAF (DDAF) which includes an area having a level of darkness being defined as qualitatively definite. In some embodiments, the other type of DAF may be questionable DAF (QDAF) which includes an area having a level of darkness being defined as qualitatively questionable. In some embodiments, the size of decreased autofluorescence (DAF) lesion may be the sum of all DDAF and QDAF. In some embodiments, DAF may be expressed as an area in square millimeters (mm²).

In some embodiments, spectral domain-optical coherence tomography (SD-OCT) is used for evaluation of ocular disease or visual function. In some embodiments, SD-OCT provides in vivo cross-sectional and enface visualization of the retina and the RPE. In some embodiments, SD-OCT is used to detect the presence of RPE atrophy. In some embodiments, SD-OCT is used to evaluate ellipsoid zone (EZ) defect width. In some embodiments, the EZ line provides information on the integrity of the photoreceptors. In some embodiments, the EZ line provides useful information in early disease. In some embodiments, an image of SD-OCT is used to evaluate the central subfield retina thickness (CST). In some embodiments, an image of SD-OCT is used to evaluate the middle subfield retina thickness (MST). In some embodiments, an image of SD-OCT is used to evaluate the outer subfield retina thickness (OST).

In some embodiments, microperimetry is used for evaluation of ocular disease or visual function. In some embodiments, microperimetry is used to measure the mean retinal sensitivity. In some embodiments, retinal sensitivity may be a level of retina response to a light stimulus. In some embodiments, microperimetry is used to spatially map retinal sensitivity. In some embodiments, microperimetry may be a psychophysical visual function test. In some embodiments, microperimetry is used for identification of correlation of anatomic feature with visual function.

In some embodiments, a method for measuring quantitative autofluorescence (qAF) is used or evaluation of ocular disease or visual function. In some embodiments, the qAF method may incorporate an internal fluorescent reference to account for variable laser power and detector sensitivity. In some embodiments, an intensity of AF may be calculated against the calibrated internal fluorescence reference standard in the optical pathway.

### Methods for Evaluation of Visual Function Using a Composite Biomarker

In one aspect, a method of treating a subject with an ocular disease is disclosed herein. In some embodiments, the method comprises one or more steps disclosed herein. In some embodiments, a method comprises one or more steps of a different method disclosed herein. In some embodiments, one or more steps of a method disclosed herein may be combined with one or more steps of another method disclosed herein. In some embodiments, the method comprises a step of administering to the subject a treatment for an ocular disease. In some embodiments, the treatment comprises a pharmaceutical composition disclosed herein. In some embodiments, the treatment may be administered in a dosage disclosed herein. In some embodiments, the method comprises a step of performing an assay on the subject to obtain at least two variables. In some embodiments, at least one of the at least two variables relate to one or more selected from the group comprising vision, visual function, ocular anatomy, ocular physiology, and ocular pathology. In some embodiments, at least one of the at least two variables relate to one or more selected from the group comprising a change in vision, a change in visual function, a change in ocular anatomy, a change in ocular physiology, and a change in ocular pathology. In some embodiments, the assay comprises at least two assays. In some embodiments, the at least two assays may be performed at two or more time points to obtain the at least two variables. In some embodiments, the at least two assays may be performed at a first time point and a second time point. In some embodiments, the assay comprises any number of assays. In some embodiments, the assay comprises any number of assays in range 1 to 100, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or any number therebetween. In some embodiments, the number of time points of performing the assay comprises any number in range 1 to 100, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or any number therebetween. In some embodiments, at least one of the at least two variables is a variable disclosed herein. In some embodiments, the at least two variables comprises all the variables disclosed herein. In some embodiments, the method comprises a step of calculating the at least two variables to yield a composite biomarker. In some embodiments, the composite biomarker may be calculated based on the at least two variables. In some embodiments, the composite biomarker may be calculated based on a formula or model disclosed herein. In some embodiments, the method comprises a step of determining whether the treatment is therapeutically effective to treat the subject based on the composite biomarker. In some embodiments, the treatment is therapeutically effective to treat the subject when the composite biomarker exceeds a threshold value. In some embodiments, the treatment is not therapeutically effective to treat the subject when the composite biomarker does not exceed the threshold value.

In some embodiments, the method of treating a subject with an ocular disease comprises, after the step of administering a treatment, a step of performing a first assay on the subject to obtain a first set of the at least two variables at a first time point. In some embodiments, the method comprises a step of calculating the first set of the at least two variables to yield a first composite biomarker. In some embodiments, a first composite biomarker may be calculated based on the first set of the at least two variables. In some embodiments, the method comprises a step of performing a second assay on the subject to obtain a second set of the at least two variables at a second time point. In some embodiments, the method comprises a step of calculating the second set of the at least two variables to yield a second composite biomarker. In some embodiments, a second composite biomarker may be calculated based on the second set of the at least two variables. In some embodiments, the method comprises a step of determining whether the treatment is therapeutically effective to treat the subject based on the first and second composite biomarkers. In some embodiments, the method comprises a step of calculating a composite biomarker based on the first and second sets of the at least two variables. In some embodiments, the method comprises a step of determining whether the treatment is therapeutically effective to treat the subject based on the composite biomarker calculated from the first and second sets of the at least two variables. In some embodiments, the composite biomarker may be calculated from the first and second sets of the at least two variables based on a formula or model disclosed herein. In some embodiments, the method comprises a step of comparing the first and second composite biomarkers to obtain a difference between the first and second composite biomarkers. In some embodiments, the treatment is therapeutically effective to treat the subject when the difference between the first and second composite biomarkers exceeds a threshold value. In some embodiments, the treatment is not therapeutically effective to treat the subject when the difference between the first and second composite biomarkers does not exceed the threshold value.

In some embodiments, the method of treating a subject with an ocular disease comprises a step of administering to the subject a first dosage of a treatment. In some embodiments, the method comprises a step of performing an assay on the subject to obtain the at least two variables after administering the first dosage of the treatment. In some embodiments, the method comprises a step of calculating the at least two variables to yield a composite biomarker. In some embodiments, a composite biomarker may be calculated based on the at least two variables. In some embodiments, the method comprises a step of determining whether the first dosage of the treatment is therapeutically effective to treat the subject based on the composite biomarker. In some embodiments, the method comprises a step of administering to the subject a second dosage of the treatment. In some embodiments, the second dosage may be different than the first dosage when the first dosage is determined to be not therapeutically effective to treat the subject. In some embodiments, the second dosage may be the same as or about the same as the first dosage when the first dosage is determined to be therapeutically effective to treat the subject. In some embodiments, the first dosage of the treatment is therapeutically effective to treat the subject when the composite biomarker exceeds a threshold value. In some embodiments, the first dosage of the treatment is not therapeutically effective to treat the subject when the composite biomarker does not exceed the threshold value.

In some embodiments, the method comprises, after the step of the administering the first dosage of the treatment, a step of performing a first assay on the subject to obtain a first set of the at least two variables at a first time point. In some embodiments, the method comprises a step of performing a second assay on the subject to obtain a second set of the at least two variables at a second time point. In some embodiments, a first composite biomarker may be calculated based on the first set of the at least two variables. In some embodiments, a second composite biomarker may be calculated based on the second set of the at least two variables. In some embodiments, the method comprises a step of determining whether the treatment is therapeutically effective to treat the subject based on the first and second composite biomarkers. In some embodiments, the method comprises a step of calculating a composite biomarker based on the first and second sets of the at least two variables. In some embodiments, the method comprises a step of determining whether the treatment is therapeutically effective to treat the subject based on the composite biomarker calculated from the first and second sets of the at least two variables. In some embodiments, the composite biomarker may be calculated from the first and second sets of the at least two variables based on a formula or model disclosed herein. In some embodiments, the method comprises a step of administering to the subject a second dosage of the treatment. In some embodiments, the second dosage may be different than the first dosage when the first dosage is determined to be not therapeutically effective to treat the subject. In some embodiments, the second dosage may be the same as or about the same as the first dosage when the first dosage is determined to be therapeutically effective to treat the subject. In some embodiments, the method comprises a step of comparing the first and second composite biomarkers to obtain a difference between the first and second composite biomarkers. In some embodiments, the treatment is therapeutically effective to treat the subject when the difference between the first and second composite biomarkers exceeds a threshold value. In some embodiments, the treatment is not therapeutically effective to treat the subject when the difference between the first and second composite biomarkers does not exceed the threshold value. In some embodiments, the treatment is therapeutically effective to treat the subject when the composite biomarker calculated from the first and second sets of the at least two variables exceed s a threshold value. In some embodiments, the treatment is not therapeutically effective to treat the subject when the composite biomarker calculated from the first and second sets of the at least two variables does not exceed the threshold value.

In some embodiments, the method of treating a subject with an ocular disease comprises, before the step of administering a treatment, a step of performing a first assay on the subject at a first time point to obtain a first set of the at least two variables disclosed herein. In some embodiments, the method comprises, before the step of administering a treatment, a step of calculating the first set of the at least two variables to yield a first composite biomarker. In some embodiments, the method comprises the step of administering the treatment to the subject. In some embodiments, the method comprises, after the step of the administering the treatment, a step of performing a second assay on the subject at a second time point to obtain a second set of the at least two variables. In some embodiments, the method comprises a step of calculating the second set of the at least two variables to yield a second composite biomarker. In some embodiments, the second composite biomarker may be calculated based on the second set of the at least two variables. In some embodiments, the method comprises a step of determining whether the treatment is therapeutically effective to treat the subject based on the first and second composite biomarkers. In some embodiments, the method comprises a step of comparing the first and second composite biomarkers to obtain a difference between the first and second composite biomarkers. In some embodiments, the treatment is therapeutically effective to treat the subject when the difference between the first and second composite biomarkers exceeds a threshold value. In some embodiments, the treatment is not therapeutically effective to treat the subject when the difference between the first and second composite biomarkers does not exceed the threshold value.

In some embodiments, the method of treating a subject with an ocular disease comprises, before the step of administering a treatment, a step of performing a first assay on the subject to obtain a first set of the at least two variables disclosed herein. In some embodiments, the method comprises a step of calculating the first set of the at least two variables to yield a first composite biomarker. In some embodiments, the method comprises, before the step of administering a treatment, a step of performing a second assay on the subject at a second time point to obtain a second set of the at least two variables. In some embodiments, the method comprises a step of calculating the second set of the at least two variables to yield a second composite biomarker. In some embodiments, the method comprises a step of comparing the first and second composite biomarkers to obtain a difference between the first and second composite biomarkers. In some embodiments, the method comprises a step of calculating a fifth composite biomarker based on the first and second sets of the at least two variables. In some embodiments, the method comprises the step of administering the treatment to the subject. In some embodiments, the method comprises, after the step of the administering the treatment, a step of performing a third assay on the subject at a third time point to obtain a third set of the at least two variables. In some embodiments, the method comprises, after the step of administering the treatment, a step of performing a fourth assay on the subject at a fourth time point to obtain a fourth set of the at least two variables. In some embodiments, the method comprises a step of calculating the third set of the at least two variables to yield a third composite biomarker. In some embodiments, the method comprises a step of calculating the fourth set of the at least two variables to yield a fourth composite biomarker. In some embodiments, the method comprises a step of comparing the third and fourth composite biomarkers to obtain a difference between the third and fourth composite biomarkers. In some embodiments, the method comprises a step of calculating a sixth composite biomarker based on the third and fourth sets of the at least two variables. In some embodiments, the method comprises a step of determining whether the treatment is therapeutically effective to treat the subject based on the first, second, third and fourth composite biomarkers. In some embodiments, the method comprises a step of comparing the difference between the first and second composite biomarkers and the difference between the third and fourth composite biomarkers to produce a differential value. In some embodiments, the treatment is therapeutically effective to treat the subject when the differential value exceeds a threshold value. In some embodiments, the treatment is not therapeutically effective to treat the subject when the differential value does not exceed the threshold value. In some embodiments, the method comprises a step of determining whether the treatment is therapeutically effective to treat the subject based on the fifth and sixth composite biomarkers. In some embodiments, the method comprises a step of comparing the fifth and sixth composite biomarkers to obtain their difference. In some embodiments, the treatment is therapeutically effective to treat the subject when the difference between the fifth and sixth composite biomarkers exceeds a threshold value. In some embodiments, the treatment is not therapeutically effective to treat the subject when the difference between the fifth and sixth composite biomarkers does not exceed the threshold value.

In some embodiments, the duration between the first and second time points of performing the first and second assays, or between the third and fourth time points of performing the third and fourth assays, may be any period of time suitable for measurement of the at least two variables disclosed herein. In some embodiments, the duration may be any period of time of about 1 day to about 1 year, e.g., about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days, 140 days, 141 days, 142 days, 143 days, 144 days, 145 days, 146 days, 147 days, 148 days, 149 days, 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 181 days, 182 days, 183 days, 184 days, 185 days, 186 days, 187 days, 188 days, 189 days, 190 days, 191 days, 192 days, 193 days, 194 days, 195 days, 196 days, 197 days, 198 days, 199 days, 200 days, 201 days, 202 days, 203 days, 204 days, 205 days, 206 days, 207 days, 208 days, 209 days, 210 days, 211 days, 212 days, 213 days, 214 days, 215 days, 216 days, 217 days, 218 days, 219 days, 220 days, 221 days, 222 days, 223 days, 224 days, 225 days, 226 days, 227 days, 228 days, 229 days, 230 days, 231 days, 232 days, 233 days, 234 days, 235 days, 236 days, 237 days, 238 days, 239 days, 240 days, 241 days, 242 days, 243 days, 244 days, 245 days, 246 days, 247 days, 248 days, 249 days, 250 days, 251 days, 252 days, 253 days, 254 days, 255 days, 256 days, 257 days, 258 days, 259 days, 260 days, 261 days, 262 days, 263 days, 264 days, 265 days, 266 days, 267 days, 268 days, 269 days, 270 days, 271 days, 272 days, 273 days, 274 days, 275 days, 276 days, 277 days, 278 days, 279 days, 280 days, 281 days, 282 days, 283 days, 284 days, 285 days, 286 days, 287 days, 288 days, 289 days, 290 days, 291 days, 292 days, 293 days, 294 days, 295 days, 296 days, 297 days, 298 days, 299 days, 300 days, 301 days, 302 days, 303 days, 304 days, 305 days, 306 days, 307 days, 308 days, 309 days, 310 days, 311 days, 312 days, 313 days, 314 days, 315 days, 316 days, 317 days, 318 days, 319 days, 320 days, 321 days, 322 days, 323 days, 324 days, 325 days, 326 days, 327 days, 328 days, 329 days, 330 days, 331 days, 332 days, 333 days, 334 days, 335 days, 336 days, 337 days, 338 days, 339 days, 340 days, 341 days, 342 days, 343 days, 344 days, 345 days, 346 days, 347 days, 348 days, 349 days, 350 days, 351 days, 352 days, 353 days, 354 days, 355 days, 356 days, 357 days, 358 days, 359 days, 360 days, 361 days, 362 days, 363 days, 364 days, or 365 days, or any period of time therebetween. In some embodiments, the duration may be any period of time of about 1 week to about 530 weeks, e.g., about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, 56 weeks, 57 weeks, 58 weeks, 59 weeks, 60 weeks, 61 weeks, 62 weeks, 63 weeks, 64 weeks, 65 weeks, 66 weeks, 67 weeks, 68 weeks, 69 weeks, 70 weeks, 71 weeks, 72 weeks, 73 weeks, 74 weeks, 75 weeks, 76 weeks, 77 weeks, 78 weeks, 79 weeks, 80 weeks, 81 weeks, 82 weeks, 83 weeks, 84 weeks, 85 weeks, 86 weeks, 87 weeks, 88 weeks, 89 weeks, 90 weeks, 91 weeks, 92 weeks, 93 weeks, 94 weeks, 95 weeks, 96 weeks, 97 weeks, 98 weeks, 99 weeks, 100 weeks, 101 weeks, 102 weeks, 103 weeks, 104 weeks, 105 weeks, 106 weeks, 107 weeks, 108 weeks, 109 weeks, 110 weeks, 111 weeks, 112 weeks, 113 weeks, 114 weeks, 115 weeks, 116 weeks, 117 weeks, 118 weeks, 119 weeks, 120 weeks, 121 weeks, 122 weeks, 123 weeks, 124 weeks, 125 weeks, 126 weeks, 127 weeks, 128 weeks, 129 weeks, 130 weeks, 131 weeks, 132 weeks, 133 weeks, 134 weeks, 135 weeks, 136 weeks, 137 weeks, 138 weeks, 139 weeks, 140 weeks, 141 weeks, 142 weeks, 143 weeks, 144 weeks, 145 weeks, 146 weeks, 147 weeks, 148 weeks, 149 weeks, 150 weeks, 151 weeks, 152 weeks, 153 weeks, 154 weeks, 155 weeks, 156 weeks, 157 weeks, 158 weeks, 159 weeks, 160 weeks, 161 weeks, 162 weeks, 163 weeks, 164 weeks, 165 weeks, 166 weeks, 167 weeks, 168 weeks, 169 weeks, 170 weeks, 171 weeks, 172 weeks, 173 weeks, 174 weeks, 175 weeks, 176 weeks, 177 weeks, 178 weeks, 179 weeks, 180 weeks, 181 weeks, 182 weeks, 183 weeks, 184 weeks, 185 weeks, 186 weeks, 187 weeks, 188 weeks, 189 weeks, 190 weeks, 191 weeks, 192 weeks, 193 weeks, 194 weeks, 195 weeks, 196 weeks, 197 weeks, 198 weeks, 199 weeks, 200 weeks, 201 weeks, 202 weeks, 203 weeks, 204 weeks, 205 weeks, 206 weeks, 207 weeks, 208 weeks, 209 weeks, 210 weeks, 211 weeks, 212 weeks, 213 weeks, 214 weeks, 215 weeks, 216 weeks, 217 weeks, 218 weeks, 219 weeks, 220 weeks, 221 weeks, 222 weeks, 223 weeks, 224 weeks, 225 weeks, 226 weeks, 227 weeks, 228 weeks, 229 weeks, 230 weeks, 231 weeks, 232 weeks, 233 weeks, 234 weeks, 235 weeks, 236 weeks, 237 weeks, 238 weeks, 239 weeks, 240 weeks, 241 weeks, 242 weeks, 243 weeks, 244 weeks, 245 weeks, 246 weeks, 247 weeks, 248 weeks, 249 weeks, 250 weeks, 251 weeks, 252 weeks, 253 weeks, 254 weeks, 255 weeks, 256 weeks, 257 weeks, 258 weeks, 259 weeks, 260 weeks, 261 weeks, 262 weeks, 263 weeks, 264 weeks, 265 weeks, 266 weeks, 267 weeks, 268 weeks, 269 weeks, 270 weeks, 271 weeks, 272 weeks, 273 weeks, 274 weeks, 275 weeks, 276 weeks, 277 weeks, 278 weeks, 279 weeks, 280 weeks, 281 weeks, 282 weeks, 283 weeks, 284 weeks, 285 weeks, 286 weeks, 287 weeks, 288 weeks, 289 weeks, 290 weeks, 291 weeks, 292 weeks, 293 weeks, 294 weeks, 295 weeks, 296 weeks, 297 weeks, 298 weeks, 299 weeks, 300 weeks, 301 weeks, 302 weeks, 303 weeks, 304 weeks, 305 weeks, 306 weeks, 307 weeks, 308 weeks, 309 weeks, 310 weeks, 311 weeks, 312 weeks, 313 weeks, 314 weeks, 315 weeks, 316 weeks, 317 weeks, 318 weeks, 319 weeks, 320 weeks, 321 weeks, 322 weeks, 323 weeks, 324 weeks, 325 weeks, 326 weeks, 327 weeks, 328 weeks, 329 weeks, 330 weeks, 331 weeks, 332 weeks, 333 weeks, 334 weeks, 335 weeks, 336 weeks, 337 weeks, 338 weeks, 339 weeks, 340 weeks, 341 weeks, 342 weeks, 343 weeks, 344 weeks, 345 weeks, 346 weeks, 347 weeks, 348 weeks, 349 weeks, 350 weeks, 351 weeks, 352 weeks, 353 weeks, 354 weeks, 355 weeks, 356 weeks, 357 weeks, 358 weeks, 359 weeks, 360 weeks, 361 weeks, 362 weeks, 363 weeks, 364 weeks, 365 weeks, 366 weeks, 367 weeks, 368 weeks, 369 weeks, 370 weeks, 371 weeks, 372 weeks, 373 weeks, 374 weeks, 375 weeks, 376 weeks, 377 weeks, 378 weeks, 379 weeks, 380 weeks, 381 weeks, 382 weeks, 383 weeks, 384 weeks, 385 weeks, 386 weeks, 387 weeks, 388 weeks, 389 weeks, 390 weeks, 391 weeks, 392 weeks, 393 weeks, 394 weeks, 395 weeks, 396 weeks, 397 weeks, 398 weeks, 399 weeks, 400 weeks, 401 weeks, 402 weeks, 403 weeks, 404 weeks, 405 weeks, 406 weeks, 407 weeks, 408 weeks, 409 weeks, 410 weeks, 411 weeks, 412 weeks, 413 weeks, 414 weeks, 415 weeks, 416 weeks, 417 weeks, 418 weeks, 419 weeks, 420 weeks, 421 weeks, 422 weeks, 423 weeks, 424 weeks, 425 weeks, 426 weeks, 427 weeks, 428 weeks, 429 weeks, 430 weeks, 431 weeks, 432 weeks, 433 weeks, 434 weeks, 435 weeks, 436 weeks, 437 weeks, 438 weeks, 439 weeks, 440 weeks, 441 weeks, 442 weeks, 443 weeks, 444 weeks, 445 weeks, 446 weeks, 447 weeks, 448 weeks, 449 weeks, 450 weeks, 451 weeks, 452 weeks, 453 weeks, 454 weeks, 455 weeks, 456 weeks, 457 weeks, 458 weeks, 459 weeks, 460 weeks, 461 weeks, 462 weeks, 463 weeks, 464 weeks, 465 weeks, 466 weeks, 467 weeks, 468 weeks, 469 weeks, 470 weeks, 471 weeks, 472 weeks, 473 weeks, 474 weeks, 475 weeks, 476 weeks, 477 weeks, 478 weeks, 479 weeks, 480 weeks, 481 weeks, 482 weeks, 483 weeks, 484 weeks, 485 weeks, 486 weeks, 487 weeks, 488 weeks, 489 weeks, 490 weeks, 491 weeks, 492 weeks, 493 weeks, 494 weeks, 495 weeks, 496 weeks, 497 weeks, 498 weeks, 499 weeks, 500 weeks, 501 weeks, 502 weeks, 503 weeks, 504 weeks, 505 weeks, 506 weeks, 507 weeks, 508 weeks, 509 weeks, 510 weeks, 511 weeks, 512 weeks, 513 weeks, 514 weeks, 515 weeks, 516 weeks, 517 weeks, 518 weeks, 519 weeks, 520 weeks, 521 weeks, 522 weeks, 523 weeks, 524 weeks, 525 weeks, 526 weeks, 527 weeks, 528 weeks, 529 weeks, or 530 weeks, or any period of time therebetween. In some embodiments, the duration disclosed above may be applicable to any two time points in any method disclosed herein that includes a step of performing at least two assays to obtain at least two sets of the least two variables disclosed herein.

In some embodiments, the method of treating a subject with an ocular disease comprises, before the step of administering a treatment, a process of selecting a subject for a treatment. In some embodiments, the method comprises a step of performing an assay on the subject to obtain the at least two variables disclosed herein. In some embodiments, the method comprises a step of calculating the at least two variables to yield a composite biomarker. In some embodiments, the method comprises a step of determining whether to select the subject for a treatment based on the composite biomarker. In some embodiments, the method comprises a step of selecting the subject for a treatment when the composite biomarker exceeds a threshold value. In some embodiments, the method comprises a step of not selecting the subject for the treatment when the composite biomarker does not exceed the threshold value. In some embodiments, the method comprises a step of administering to the subject the treatment when the subject is selected for the treatment. In some embodiments, the method comprises a step of not administering to the subject a treatment when the subject is not selected for the treatment.

In some embodiments, the process of selecting a subject for a treatment comprises a step of performing a first assay on the subject to obtain a first set of the at least two variables. In some embodiments, the process of selecting a subject for a treatment comprises a step of performing a second assay on the subject to obtain a second set of the at least two variables. In some embodiments, the process of selecting a subject for a treatment comprises calculating the first set and second sets of the at least two variables to obtain a first composite biomarker and a second composite biomarker. In some embodiments, the process of selecting a subject comprises calculating the first and second sets of the at least two variables to obtain a composite biomarker. In some embodiments, the process of selecting a subject for a treatment comprises determining whether to select the subject for a treatment based on the first and second composite biomarkers. In some embodiments, the process of selecting a subject for a treatment comprises determining whether to select the subject for a treatment based on the composite biomarker calculated from the first and second sets of the at least two variables. In some embodiments, the process of selecting a subject for a treatment comprises comparing the first and second composite biomarkers to obtain their difference. In some embodiments, the process of selecting a subject for a treatment comprises selecting the subject for a treatment when the difference between the first and second composite biomarkers exceeds a threshold value. In some embodiments, the process of selecting a subject for a treatment comprises not selecting the subject for a treatment when the difference between the first and second composite biomarkers does not exceeds the threshold value. In some embodiments, the method comprises selecting the subject for a treatment when the composite biomarker calculated from the first and second sets of the at least two variables exceeds a threshold value. In some embodiments, the method comprises a step of not selecting the subject for the treatment when the composite biomarker calculated from the first and second sets of the at least two variables does not exceed the threshold value. In some embodiments, the threshold value of the difference between the first and second composite biomarkers may be the same or different than the threshold value of the composite biomarker calculated from the first and second sets of the at least two variables.

In some embodiments, the process of selecting a subject for a treatment for an ocular disease may be combined with one or more steps of a method disclosed herein including, but not limited to, a process of determining whether a treatment or a dosage of a treatment is therapeutically effective to treat a subject based on one or more composite biomarkers calculated from the at least two variables disclosed herein, and a process of administering to a subject a therapeutically effective dosages of a treatment based on one or more composite biomarkers calculated from the at least two variables measured after the subject received a certain dosage of the treatment.

In some embodiments, one or more steps of a method disclosed herein is used to monitor the outcome of a treatment for an ocular disease in a subject. In some embodiments, a method comprises determining whether a dosage of a treatment administered to the subject is therapeutically effective to treat the subject by calculating at least two variables of the subject disclosed herein to yield one or more composite biomarkers disclosed herein. In some embodiments, the method comprises other steps including, but not limited to, performing one or more assays on the subject to obtain at least two variables, and calculating the at least two variables of the subject to yield one or more composite biomarkers.

In some embodiments, one or more steps of a method disclosed herein is used to predict a subject's therapeutic response to a treatment for an ocular disease. In some embodiment, the method comprises predicting the subject's therapeutic response to a treatment based on one or more composite biomarkers calculated from the at least two variables disclosed herein. In some embodiments, the method comprises determining whether the subject is likely to have therapeutic response to the treatment based on the one or more composite biomarkers. In some embodiments, the subject is likely to have therapeutic response to the treatment when the composite biomarker exceeds a threshold value. In some embodiments, the subject is likely not to have therapeutic response to the treatment when the composite biomarker does not exceed the threshold value. In some embodiments, the subject is likely to have therapeutic response to the treatment when the difference between two or more composite biomarkers disclosed herein exceeds a threshold value. In some embodiments, the subject is likely not to have therapeutic response to the treatment when the difference between two or more composite biomarkers disclosed herein does not exceed the threshold value. In some embodiments, the method comprises other steps including, but not limited to, performing one or more assays on the subject to obtain at least two variables, and calculating the at least two variables of the subject to yield one or more composite biomarkers.

In some embodiments, one or more steps of a method disclosed herein is used to determine a dosage of a treatment for a subject with an ocular disease based on one or more composite biomarkers calculated from the at least two variables disclosed herein. In some embodiments, a dosage of a treatment may be determined by calculating a composite biomarker or the difference between two or more composite biomarkers. In some embodiments, the dosage of a treatment correlates with the value of a composite biomarker or the difference in value between two or more composite biomarkers. In some embodiments, the at least two variables disclosed herein are obtained after administering a treatment to the subject. In some embodiments, the at least two variables disclosed herein are obtained before administering a treatment to the subject.

In some embodiments, one or more steps of a method disclosed herein is used to monitor the progression of an ocular disease in a subject based on one or more composite biomarkers calculated from the at least two variables disclosed herein. In some embodiments, a state of the ocular disease may be determined by calculating a composite biomarker or the difference between two or more composite biomarkers. In some embodiments, the state of the ocular disease correlates with the value of a composite biomarker or the difference in value between two or more composite biomarkers. In some embodiments, the at least two variables disclosed herein are obtained after administering a treatment to the subject. In some embodiments, the at least two variables disclosed herein are obtained before administering a treatment to the subject.

In some embodiments, the at least two variables comprises any number of variables suitable for the method disclosed herein. In some embodiments, the at least two variables comprises a number of variables in the range 2 to 50, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or any number therebetween. In some embodiments, the at least one of the at least two variables disclosed herein relate to one or more selected from the group comprising visual acuity (VA), a score of functional visual questionnaire, and a measurement obtained from an imaging technology disclosed herein. In some embodiments, the at least one of the at least two variables relate a change in ocular structure, a change in ocular pathology, a change in retinal atrophy, a change in macular atrophy, a change in macular degermation, or a combination thereof. In some instances, the at least two variables comprises a treatment (e.g., drug type, dose, frequency, administration method), such as treatment with an RBP4 inhibitor. In some instances, a biomarker provided herein predicts a therapeutic response to a treatment.

In some embodiments, the functional visual questionnaire score comprises an average score of functional visual questionnaire. In some embodiments, the imaging technology comprises one or more imaging technologies disclosed herein. In some embodiments, the at least one of the at least two variables relate to one or more selected from the group comprising a change in visual acuity, a change in functional visual questionnaire score, and a change in measurement obtained from an imaging technology. In some embodiments, a change in functional visual questionnaire score comprises a change in an average score of functional visual questionnaire. In some embodiments, the imaging technology comprises a technology for evaluating ocular structure or visual function. In some embodiments, the imaging technology comprises a technology for detecting or quantifying a change in ocular structure or visual function. In some embodiments, the at least one of the at least two variables relate to a change in visual acuity, a change in average score of functional visual questionnaire, and a change in a measurement obtained from an imaging technology for detecting or quantifying a change in optic atrophy or visual function. In some embodiments, the at least two variables relate to a change in visual acuity, a change in average score of functional visual questionnaire, and a change in a measurement obtained from an imaging technology for detecting or quantifying a change in optic atrophy or visual function.

In some embodiments, the at least two variables relate to visual acuity and average score of functional visual questionnaire. In some embodiments, the at least two variables relate to a change in visual acuity and a change in average score of functional visual questionnaire. In some embodiments, the at least two variables relate to visual acuity and a measurement obtained from an imaging technology for detecting or quantifying a change in optic atrophy or visual function. In some embodiments, the at least two variables relate to a change in visual acuity and a change in a measurement obtained from an imaging technology for detecting or quantifying a change in optic atrophy or visual function. In some embodiments, the at least two variables relate to average score of functional visual questionnaire and a measurement obtained from an imaging technology for detecting or quantifying a change in optic atrophy or visual function. In some embodiments, the at least two variables relate to a change in average score of functional visual questionnaire and a change in a measurement obtained from an imaging technology for detecting or quantifying a change in optic atrophy or visual function. In some embodiments, the at least two variables all relate to visual acuity. In some embodiments, the at least two variables all relate to a change in visual acuity. In some embodiments, the at least two variables all relate to average score of functional visual questionnaire. In some embodiments, the at least two variables all relate to a change in average score of functional visual questionnaire. In some embodiments, the at least two variables all relate to a measurement obtained from an imaging technology for detecting or quantifying a change in optic atrophy or visual function. In some embodiments, the at least two variables all relate to a change in a measurement obtained from an imaging technology for detecting or quantifying a change in optic atrophy or visual function.

In some embodiments, two of the at least two variables correlates with each other. In some embodiments, a correlation between two of the at least two variables may be expressed using a correlation coefficient. In some embodiments, a correlation coefficient formula is used to evaluate the strength of a correlation relationship between two of the at least two variables. In some embodiments, the correlation coefficient formula may generate a value between −1 and 1. In some embodiments, the value of one (1) may indicate a strong positive relationship. In some embodiments, the value of minus one (−1) may indicate a strong negative relationship. In some embodiments, the value of zero (0) may indicate no relationship. In some embodiments, one of the at least two variables correlates with visual acuity. In some embodiments, one of the at least two variables correlates with a change in visual acuity. In some embodiments, one of the at least two variables correlates with a score of functional visual questionnaire. In some embodiments, one of the at least two variables correlates with a change in functional visual questionnaire score. In some embodiments, one of the at least two variables correlates with an average score of functional visual questionnaire. In some embodiments, one of the at least two variables correlates with a change in average score of functional visual questionnaire. In some embodiments, one of the at least two variables correlates with a measurement obtained from an imaging technology for detecting or quantifying a change in optic atrophy or visual function. In some embodiments, one of the at least two variables correlates with a change in measurement obtained from an imaging technology for detecting or quantifying a change in optic atrophy or visual function. In some embodiments, the at least two variables comprises two variables of visual acuity that correlate with each other. In some embodiments, the at least two variables comprises two variables of a change in visual acuity that correlate with each other. In some embodiments, the at least two variables comprises two variables of average score of functional visual questionnaire that correlate with each other. In some embodiments, the at least two variables comprises two variables of a change in average score of functional visual questionnaire that correlate with each other. In some embodiments, the at least two variables comprises two variables of a measurement obtained from an imaging technology for detecting or quantifying a change in optic atrophy or visual function that correlate with each other. In some embodiments, the at least two variables comprises two variables of a change in a measurement obtained from an imaging technology for detecting or quantifying a change in optic atrophy or visual function that correlate with each other. In some embodiments, all of the at least two variables correlates with visual acuity. In some embodiments, all of the at least two variables correlates with a change in visual acuity. In some embodiments, all of the at least two variables correlates with average score of functional visual questionnaire. In some embodiments, all of the at least two variables correlates with a change in average score of functional visual questionnaire. In some embodiments, all of the at least two variables correlates with a measurement obtained from an imaging technology for detecting or quantifying a change in optic atrophy or visual function. In some embodiments, all of the at least two variables correlates with a change in measurement obtained from an imaging technology for detecting or quantifying a change in optic atrophy or visual function. In some embodiments, one of the at least two variables correlates with two selected from the group comprising visual acuity, average score of functional visual questionnaire, and a measurement obtained from an imaging technology for detecting or quantifying a change in optic atrophy or visual function. In some embodiments, one of the at least two variables correlates with two selected from the group comprising a change in visual acuity, a change in average score of functional visual questionnaire, and a change in measurement obtained from an imaging technology for detecting or quantifying a change in optic atrophy or visual function. In some embodiments, one of the at least two variables correlates with each of visual acuity, average score of functional visual questionnaire, and a measurement obtained from an imaging technology for detecting or quantifying a change in optic atrophy or visual function. In some embodiments, one of the at least two variables correlates with each of a change in visual acuity, a change in average score of functional visual questionnaire, and a change in measurement obtained from an imaging technology for detecting or quantifying a change in optic atrophy or visual function. In some embodiments, one of the at least two variables correlates with a state of the ocular disease. In some embodiments, one of the at least two variables correlates progression of the ocular disease.

In some embodiments, the at least two variables comprises a value obtained from a logarithm of the minimum angle of resolution (log MAR) chart, a Snellen chart, a best corrected visual acuity (BCVA) test, an early treatment diabetic retinopathy study (ETDRS) letter test, or a combination thereof. In some embodiments, the at least two variables comprises two values obtained from a logarithm of the minimum angle of resolution (log MAR) chart, a Snellen chart, a best corrected visual acuity (BCVA) test, and an early treatment diabetic retinopathy study (ETDRS) letter test.

In some embodiments, the at least two variables comprises a score of functional visual questionnaire comprising one or more questions that relate to one or more from the group comprising the subject's confidence of moving to different places in the daytime, the subject's confidence of moving to different places in the nighttime, the subject's ability to recognize people, the subject's ability to take part in sport, the subject's ability to get information, the subject's ability for social interaction, and the subject's ability for work efficiency. In some embodiments, the at least two variables comprises a score of functional visual questionnaire comprising one or more questions that relate to one or more from the group comprising the subject's confidence of moving to different places in the daytime or nighttime, the subject's ability to recognize people, to take part in sport or to get information, and the subject's ability for social interaction or work efficiency. In some embodiments, each of the one or more questions may be scored with respect to the subject. In some embodiments, an average score are obtained from two or more questions of a visual questionnaire test, two or more functional visual questionnaire comprising one or more questions, or both. In some embodiments, the score scale of a visual questionnaire may be any range between two numbers. In some embodiments, the score scale may be 0 to 4. In some embodiments, an answer of “NEVER” to a question is assigned a score according to the score scale. In some embodiments, an answer of “ALMOST NEVER” to a question is assigned a score according to the score scale. In some embodiments, an answer of “SOMETIMES” to a question is assigned a score according to the score scale. In some embodiments, an answer of “ALMOST ALWAYS” to a question is assigned a score according to the score scale. In some embodiments, an answer of “ALWAYS” to a question is assigned a score according to the score scale. In some embodiments, the answers, “NEVER,” “ALMOST NEVER,” “SOMETIMES,” “ALMOST ALWAYS,” and “ALWAYS,” to a question is assigned a score of 0, 1, 2, 3, 4, respectively, according to the 0-4 score scale. In some embodiments, the answers, “NEVER,” “ALMOST NEVER,” “SOMETIMES,” “ALMOST ALWAYS,” and “ALWAYS,” to a question is assigned a score of 4, 3, 2, 1, 0, respectively, according to the 0-4 score scale. In some embodiments, the answers, “NEVER,” “ALMOST NEVER,” “SOMETIMES,” “ALMOST ALWAYS,” and “ALWAYS,” to a question is assigned a score according to any scale.

In some embodiments, the at least two variables comprises two or more values obtained from microperimetry (MP), perimetry, or both. In some embodiments, the MP comprises measuring a spatial map of retinal sensitivity of the subject. In some embodiments, the MP comprises measuring a mean retina sensitivity of the subject, a change thereof, or both. In some embodiments, the at least two variables comprises two or more values obtained from two or more selected from the group comprising fundus photography, fundus angiography (FA), fundus autofluorescence (FAF), spectral domain-optical coherence tomography (SD-OCT), quantitative Autofluorescence (qAF), infrared (IR) imaging, optical coherence tomography-angiography (OCT-A), and widefield imaging. In some embodiments, the at least two variables comprises at least two values each obtained from one of the group comprising fundus photography, fundus angiography (FA), fundus autofluorescence (FAF), spectral domain-optical coherence tomography (SD-OCT), quantitative Autofluorescence (qAF), infrared (IR) imaging, optical coherence tomography-angiography (OCT-A), and widefield imaging. In some embodiments, the at least two variables comprises two or more values selected from the group comprising an area of questionable decreased autofluorescence (QDAF), an area of definite decreased autofluorescence (DDAF), an area of decreased autofluorescence (DAF), a retina thickness, an ellipsoid zone (EZ) defect width, a central subfield retina thickness (CST), an outer subfield retina thickness (OST), and a middle subfield retina thickness (MST). In some embodiments, the at least two variables comprises two or more values selected from the group comprising a change in area of questionable decreased autofluorescence (QDAF), a change in area of definite decreased autofluorescence (DDAF), a change in area of decreased autofluorescence (DAF), a change in retina thickness, a change in ellipsoid zone (EZ) defect width, a change in central subfield retina thickness (CST), a change in outer subfield retina thickness (OST), and a change in middle subfield retina thickness (MST). In some embodiments, the at least two variables comprises DAF and EZ defect width. In some embodiments, the at least two variables comprises a change in DAF, and a change in EZ defect width. In some embodiments, an area of decreased autofluorescence (DAF) comprises an area of questionable decreased autofluorescence (QDAF) and an area of definite decreased autofluorescence (DDAF). In some embodiments, a change in area of decreased autofluorescence (DAF) comprises a change in area of questionable decreased autofluorescence (QDAF) and a change in area of definite decreased autofluorescence (DDAF). In some embodiments, the at least two variables comprises two or more selected from the group comprising visual acuity, QDAF, DAF, and EZ defect width. In some embodiments, the at least two variables comprises two or more selected from the group comprising a change in visual acuity, a change in QDAF, a change in DAF, and a change in EZ defect width. In some embodiments, the at least two variables comprises visual acuity, QDAF, DAF, and EZ defect width. In some embodiments, the at least two variables comprises a change in visual acuity, a change in QDAF, a change in DAF, and a change in EZ defect width. In some embodiments, EZ defect width correlates with visual acuity. In some embodiments, a change in EZ defect width correlates with a change in visual acuity. In some embodiments, EZ defect width correlates with QDAF. In some embodiments, a change in EZ defect width correlates with a change in QDAF. In some embodiments, EZ defect width correlates with DAF. In some embodiments, a change in EZ defect width correlates with a change in DAF. In some embodiments, visual acuity correlates with QDAF. In some embodiments, QDAF comprises a measure of autofluorescence. In some embodiments, a change in visual acuity correlates with a change in QDAF. In some embodiments, a change in QDAF comprises a measure of autofluorescence.

In some embodiments, EZ defect width correlates with visual acuity, QDAF, and DAF. In some embodiments, a change in EZ defect width correlates with a change in visual acuity, a change in QDAF, and a change in DAF. In some embodiments, EZ defect width correlates with visual acuity, QDAF, and DAF with a correlation coefficient of more than about 0.8 and a p value of less than about 0.05. In some embodiments, a change in EZ defect width correlates with a change in visual acuity, a change in QDAF, and a change in DAF with a correlation coefficient of more than about 0.8 and a p value of less than about 0.05. In some embodiments, visual acuity correlates with EZ defect width and QDAF. In some embodiments, a change in visual acuity correlates with a change in EZ defect width and a change in QDAF. In some embodiments, visual acuity correlates with EZ defect width and QDAF with a p value of less than about 0.05. In some embodiments, a change in visual acuity correlates with a change in EZ defect width and a change in QDAF with a p value of less than about 0.05. In some embodiments, the at least two variables are obtained from both the right and left eyes of the subject. In some embodiments, the at least two variables are obtained from at least two subjects with different gender, ethnicity, age, or a combination thereof. In some embodiments, the at least two variables are obtained from a clinical trial. In some embodiments, the at least two variables are obtained from at least two phases of a clinical trial.

In some embodiments, the one or more composite biomarkers disclosed herein is used a surrogate biomarker or endpoint for detecting or predicting visual functional improvement. In some embodiments, a method disclosed herein is used for detecting and/or predicting visual function change in clinical practice or clinical trial. In some embodiments, a method disclosed herein is used for detecting and/or predicting visual improvement in clinical practice or clinical trial. In some embodiments, a method disclosed herein is used to develop an algorithm or software. In some embodiments, a method disclosed herein may be applied to a medical device, virtual reality goggle, metaverse, video game, or a combination thereof.

In some embodiments, a method disclosed herein may be applied to detect and measure a multidimensional visual function. In some embodiments, the multidimensional visual function comprises spatial awareness, eye-body coordination, or visual motion. In some embodiments, a method disclosed herein may be applied to detect and measure visual loss or improvement as a multidimensional or multifactorial measurement. In some embodiments, a method disclosed herein may be applied to develop an algorithm, software, or a scoring system for diagnosis of ocular disease, detection of visual function improvement or loss, or detection of therapeutic response during clinical trial.

In some embodiments, a method disclosed herein may be applied to develop a prediction model related to the variable disclosed herein. In some embodiments, the prediction model may be constructed with a link function based on the magnitude of the variance of each response variable as a function of its predicted value. In some embodiments, the prediction model comprises three components. In some embodiments, the three components comprises a predictor based on a function of regressors (ηᵢ). In some embodiments, the three components comprises a model structure best fitting the conditional distribution of the response variable, Yᵢ, given the values of the explanatory variables in the model. In some embodiments, the three components comprises a smooth and invertible link function g(⋅), which transforms the expectation of the response variable, μᵢ=E(Yᵢ), to the predictor: g(μᵢ)=ηᵢ=α+β₁ Xᵢ₁+β₂ Xᵢ₂+ . . . + βₖ Xᵢₖ. In some embodiments, the prediction model may be constructed using visual acuity (VA) and EZ width as follows: VA=−0.0896 EZ WIDTH+0.0701, Z-score=−2.211, P value=0.027, and CI 95% [−0.169, −0.010]. In some embodiments, another univariate or multivariate model may be constructed using visual acuity and EZ width.

In some embodiments, a method disclosed herein is used for any ocular disease. In some embodiments, the ocular disease comprises one or more selected from the group comprising maculopathy, retinopathy, retina atrophy, macular atrophy, retina degeneration, macular degeneration, age-related macular degeneration (AMD), RP (Retinitis pigmentosa), an ABCA4 gene mutation, Stargardt's Disease (STGD), and STGD 1. In some embodiments, the ocular disease comprises retina degeneration. In some embodiments, the ocular disease comprises macular degeneration. In some embodiments, the ocular disease comprises AMD. In some embodiments, the ocular disease comprises STGD. In some embodiments, the ocular disease comprises STGD 1.

In some embodiments, the ocular disease may be diagnosed according to on one or more factors disclosed herein. In some embodiments, a sample from the subject with the ocular disease may have an expression level of retinol binding protein 4 (RBP4) of at least a threshold value. In some embodiments, the threshold value of RBP4 may be any value for the diagnosis of a macular degeneration disease disclosed herein. In some embodiments, the threshold level of RBP4 is at least g/mL. In some embodiments, the threshold level of RBP4 is at least 30 μg/mL. In some embodiments, the threshold level of RBP4 is at least 35 μg/mL. In some embodiments, the threshold value is about 25 μg/mL to about 100 μg/mL. In some embodiments, the threshold value is about 25 g/mL to about 30 μg/mL, about 25 μg/mL to about 35 μg/mL, about 25 μg/mL to about 40 μg/mL, about 25 μg/mL to about 50 μg/mL, about g/mL to about g/mL, about g/mL to about μg/mL, about 25 μg/mL to about 80 μg/mL, about 25 μg/mL to about 90 μg/mL, about 25 μg/mL to about 100 μg/mL, about 30 μg/mL to about 35 μg/mL, about 30 μg/mL to about 40 μg/mL, about 30 g/mL to about 50 μg/mL, about 30 μg/mL to about 60 μg/mL, about 30 μg/mL to about 70 μg/mL, about 30 μg/mL to about 80 μg/mL, about 30 μg/mL to about 90 μg/mL, about 30 μg/mL to about 100 μg/mL, about 35 μg/mL to about 40 μg/mL, about 35 μg/mL to about 50 μg/mL, about 35 g/mL to about 60 μg/mL, about 35 μg/mL to about 70 μg/mL, about 35 μg/mL to about 80 μg/mL, about 35 μg/mL to about 90 μg/mL, or about 35 μg/mL to about 100 μg/mL. In some embodiments, the threshold value is about 25 μg/mL, about 30 μg/mL, or about 35 μg/mL. In some embodiments, the threshold value is at least about 25 μg/mL, about 30 μg/mL, or about 35 μg/mL. In some embodiments, the threshold level of RBP4 disclosed herein may be applicable to an ocular disease disclosed herein. In some embodiments, the threshold level of RBP4 disclosed herein may be applicable to AMD, STGD, or STGD 1. In some embodiments, the threshold value is at most about g/mL, about 35 μg/mL, about 40 μg/mL, about 50 μg/mL, about 60 μg/mL, about 70 μg/mL, about 80 μg/mL, about 90 μg/mL, or about 100 μg/mL. In some embodiments, the level of RBP4 in the subject may be assessed by an assay. Any suitable assay for measuring the level of RBP4 can be employed (e.g. antibody assay, mass spectrometry based assay (e.g. LC/MS), liquid chromatography assay (e.g. HPLC, UPLC), etc.). In some embodiments, the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. In some embodiments, the assay comprises an antibody assay. In some embodiments, the antibody assay comprises an enzyme-linked immunosorbent assay (ELISA).

In some embodiments, the threshold value of RBP4 may vary based on various characteristics of the individual. Non-limiting examples of such characteristics comprises demographic data, gender, age, medical history, previous diagnosis with an eye condition, body-mass index (BMI), height, weight, a systemic condition including obesity, hyperlipidemia, hyperglycemia, hypertension, diabetes, mellitus, cardiovascular disease, or non-alcoholic fatty liver diseases.

In some embodiments, a diagnosis of an ocular disease is made if the level of RBP4 is above the threshold value. In some embodiments, a risk score is calculated if the level of RBP4 is above the threshold value.

In some embodiments, a therapy is administered if the level of RBP4 is above the threshold value. In some embodiments, the therapy is administered to reduce the level of RBP4. In some embodiments, the therapy comprises administering a pharmaceutical composition to the individual. In some embodiments, the therapy comprises administering a pharmaceutical compound to the individual. In some embodiments, the pharmaceutical composition comprises an RBP4 inhibitor. The RBP4 inhibitor may be any of the RBP4 inhibitors provided herein.

In some embodiments, a sample from the subject with the ocular disease may have an expression level of Vitamin A of at least a threshold value. In some embodiments, the threshold level of Vitamin A is at least about 150 ng/mL. In some embodiments, the threshold level of Vitamin A is at least about 175 ng/mL. In some embodiments, the threshold level of Vitamin A is at least about 200 ng/mL. In some embodiments, the threshold level of Vitamin A is at least about 220 ng/mL. In some embodiments, the threshold level of Vitamin A is at least about 221 ng/mL. In some embodiments, the threshold level of Vitamin A is at least about 222 ng/mL. In some embodiments, the threshold level of Vitamin A is at least about 223 ng/mL. In some embodiments, the threshold level of Vitamin A is at least about 224 ng/mL. In some embodiments, the threshold level of Vitamin A is at least about 225 ng/mL. In some embodiments, the threshold level of Vitamin A is at least about 250 ng/mL. In some embodiments, the threshold level of Vitamin A is at least about 300 ng/mL. In some embodiments, the threshold level of Vitamin A is at least about 350 ng/mL. In some embodiments, the threshold level of Vitamin A is at least about 390 ng/mL. In some embodiments, the threshold level of Vitamin A is at least about 391 ng/mL. In some embodiments, the threshold level of Vitamin A is at least about 392 ng/mL. In some embodiments, the threshold level of Vitamin A is at least about 393 ng/mL. In some embodiments, the threshold level of Vitamin A is at least about 394 ng/mL. In some embodiments, the threshold level of Vitamin A is at least about 395 ng/mL. In some embodiments, the threshold level of vitamin A is about 150 ng/mL to about 500 ng/mL. In some embodiments, the threshold level of vitamin A is about 150 ng/mL to about 175 ng/mL, about 150 ng/mL to about 200 ng/mL, about 150 ng/mL to about 225 ng/mL, about 150 ng/mL to about 250 ng/mL, about 150 ng/mL to about 300 ng/mL, about 150 ng/mL to about 350 ng/mL, about 150 ng/mL to about 400 ng/mL, about 150 ng/mL to about 450 ng/mL, about 150 ng/mL to about 500 ng/mL, about 175 ng/mL to about 200 ng/mL, about 175 ng/mL to about 225 ng/mL, about 175 ng/mL to about 250 ng/mL, about 175 ng/mL to about 300 ng/mL, about 175 ng/mL to about 350 ng/mL, about 175 ng/mL to about 400 ng/mL, about 175 ng/mL to about 450 ng/mL, about 175 ng/mL to about 500 ng/mL, about 200 ng/mL to about 225 ng/mL, about 200 ng/mL to about 250 ng/mL, about 200 ng/mL to about 300 ng/mL, about 200 ng/mL to about 350 ng/mL, about 200 ng/mL to about 400 ng/mL, about 200 ng/mL to about 450 ng/mL, about 200 ng/mL to about 500 ng/mL, about 225 ng/mL to about 250 ng/mL, about 225 ng/mL to about 300 ng/mL, about 225 ng/mL to about 350 ng/mL, about 225 ng/mL to about 400 ng/mL, about 225 ng/mL to about 450 ng/mL, about 225 ng/mL to about 500 ng/mL, about 250 ng/mL to about 300 ng/mL, about 250 ng/mL to about 350 ng/mL, about 250 ng/mL to about 400 ng/mL, about 250 ng/mL to about 450 ng/mL, about 250 ng/mL to about 500 ng/mL, about 300 ng/mL to about 350 ng/mL, about 300 ng/mL to about 400 ng/mL, about 300 ng/mL to about 450 ng/mL, about 300 ng/mL to about 500 ng/mL, about 350 ng/mL to about 400 ng/mL, about 350 ng/mL to about 450 ng/mL, about 350 ng/mL to about 500 ng/mL, about 400 ng/mL to about 450 ng/mL, about 400 ng/mL to about 500 ng/mL, or about 450 ng/mL to about 500 ng/mL. In some embodiments, the threshold level of vitamin A is about 150 ng/mL, about 175 ng/mL, about 200 ng/mL, about 225 ng/mL, about 250 ng/mL, about 300 ng/mL, about 350 ng/mL, about 400 ng/mL, about 450 ng/mL, or about 500 ng/mL. In some embodiments, the threshold level of vitamin A is at least about 150 ng/mL, about 175 ng/mL, about 200 ng/mL, about 225 ng/mL, about 250 ng/mL, about 300 ng/mL, about 350 ng/mL, about 400 ng/mL, or about 450 ng/mL. In some embodiments, the threshold level of vitamin A is at most about 175 ng/mL, about 200 ng/mL, about 225 ng/mL, about 250 ng/mL, about 300 ng/mL, about 350 ng/mL, about 400 ng/mL, about 450 ng/mL, or about 500 ng/mL. In some embodiments, the threshold level of vitamin A disclosed herein may be applicable to an ocular disease disclosed herein. In some embodiments, the threshold level of vitamin A disclosed herein may be applicable to AMD, STGD, or STGD 1.

In some embodiments, the threshold value may vary based on various characteristics of the individual. Non-limiting examples of such characteristics comprises demographic data, gender, age, medical history, previous diagnosis with an eye condition, body-mass index (BMI), height, weight, a systemic condition including obesity, hyperlipidemia, hyperglycemia, hypertension, diabetes, mellitus, cardiovascular disease, or non-alcoholic fatty liver diseases.

In some embodiments, the threshold level of vitamin A depends on the age of the individual. In some embodiments, the threshold level of vitamin A is lesser for an adolescent (e.g., an individual with an age of 12-17) than for an adult (e.g., an age of 18 or older). In some embodiments, the threshold level of vitamin A for an adolescent is at least about 150 ng/mL, at least about 175 ng/mL, at least about 200 ng/mL, at least about 210 ng/mL, at least about 220 ng/mL, at least about 221 ng/mL, at least about 222 ng/mL, at least about 223 ng/mL, at least about 224 ng/mL, at least about 225 ng/mL, at least about 230 ng/mL, or at least about 250 ng/mL. In some embodiments, the threshold for an adult is at least about 300 ng/mL, at least about 325 ng/mL, at least about 350 ng/mL, at least about 375 ng/mL, at least about 380 ng/mL, at least about 385 ng/mL, at least about 390 ng/mL, at least about 391 ng/mL, at least about 392 ng/mL, at least about 393 ng/mL, at least about 394 ng/mL, at least about 395 ng/mL, or at least about 400 ng/mL.

In some embodiments, a diagnosis of an ocular disease is made if the level of vitamin A is above the threshold value. In some embodiments, a risk score is calculated if the level of vitamin A is above the threshold value.

In some embodiments, a treatment is administered if the level of vitamin A is above the threshold value. In some embodiments, the therapy is administered to reduce the level of vitamin A. In some embodiments, the therapy comprises administering a pharmaceutical composition to the individual. In some embodiments, the therapy comprises administering a pharmaceutical compound to the individual. In some embodiments, the pharmaceutical composition comprises an RBP4 inhibitor. The RBP4 inhibitor may be any of the RBP4 inhibitors provided herein.

In some embodiments, the level of vitamin A in the individual is assessed by an assay. Any suitable assay for measuring the level of vitamin A can be employed (e.g., antibody assay, mass spectrometry based assay (e.g., LC/MS), liquid chromatography assay (e.g., HPLC, UPLC), etc.). In some embodiments, the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. In some embodiments, the assay comprises an antibody assay. In some embodiments, the antibody assay comprises an enzyme-linked immunosorbent assay (ELISA). In some embodiments, the assay is a chromatographic assay. In some embodiments, the assay comprises high performance liquid chromatography (HPLC), ultra-performance liquid chromatography (UPLC), or liquid chromatography/mass spectrometry (LC-MS).

In some embodiments, the sample comprises a blood sample from the subject. The blood sample may be further processed, such as to remove impurities or to leave behind the serum and/or plasma. In some embodiments, the sample is a plasma or serum sample. In some embodiments, the sample is a plasma sample. In some embodiments, the sample is a serum sample. In some embodiments, the level is measured from plasma or serum derived from the blood sample. In some embodiments, the level is measured from plasma derived from the blood sample. In some embodiments, the level is measured from serum derived from the blood sample.

In some embodiments, a method disclosed herein comprises a step of assessing whether a subject has an ocular disease disclosed herein, is likely to develop an ocular disease disclosed herein, such as AMD or STGD. In some embodiments, the method comprises determining by an assay the presence or absence of one or more genomic variants. In some embodiments, the one or more genomic variants comprises at least one of rs4147863, rs2275029, rs1800739, rs4147857, rs4147856, rs1801555, or rs1801574. In some embodiments, the method comprises calculating a risk score for an ocular disease using the presence or absence of the one or more genomic variants. In some embodiments, the method comprises diagnosing an ocular disease using the presence or absence of the one or more genomic variants. In some embodiments, the method comprises determining by an assay the presence or absence of one or more genomic variants. In some embodiments, the one or more genomic variants comprises at least one of rs4147863, rs2275029, rs1800739, rs4147857, rs4147856, rs1801555, or rs1801574. In some embodiments, the one or more genomic variants comprises at least two of rs4147863, rs2275029, rs1800739, rs4147857, rs4147856, rs1801555, or rs1801574. In some embodiments, the one or more genomic variants comprises at least three of rs4147863, rs2275029, rs1800739, rs4147857, rs4147856, rs1801555, or rs1801574. In some embodiments, the one or more genomic variants comprises at least four of rs4147863, rs2275029, rs1800739, rs4147857, rs4147856, rs1801555, or rs1801574. In some embodiments, the one or more genomic variants comprises at least five of rs4147863, rs2275029, rs1800739, rs4147857, rs4147856, rs1801555, or rs1801574. In some embodiments, the one or more genomic variants comprises at least six of rs4147863, rs2275029, rs1800739, rs4147857, rs4147856, rs1801555, or rs1801574. In some embodiments, the one or more genomic variants comprises each of rs4147863, rs2275029, rs1800739, rs4147857, rs4147856, rs1801555, or rs1801574. In some embodiments, the one or more genomic variants comprises rs4147863. In some embodiments, the one or more genomic variants comprises rs2275029. In some embodiments, the one or more genomic variants comprises rs1800739. In some embodiments, the one or more genomic variants comprises rs4147857. In some embodiments, the one or more genomic variants comprises rs4147856. In some embodiments, the one or more genomic variants comprises rs1801555. In some embodiments, the one or more genomic variants comprises rs1801574. In some embodiments, the one or more genomic variants comprises at least one of rs3747961, rs6666652, rs1800717, rs763108716, rs185601596, rs17110761, rs6l 748519, rs1801359, rs145766145, rs76258939, rs200551567, rs754765164, rs201602424, rs564661476, rs4147831, rs6657239, rs2297632, rs1801555, rs1762114, rs55860151, rs1800549, rs3112831, rs4147830, rs2297634, or rs4847281. In some embodiments, the one or more genomic variants comprises at least five of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555, rs1762114 rs55860151, rs1800549, rs3112831, rs4147830, rs2297634, or rs4847281. In some embodiments, the one or more genomic variants comprises at least 1, 2, 3, 4, 5, 6, 7, 8, 9, 10, 11, 12, 13, 14, 15, 16, 17, 18, 19, or 20 of the genomic variants. In some embodiments, the one or more genomic variants comprises at least 1 genomic variant from Table A. In some embodiments, the one or more genomic variants comprises at least 5 genomic variants from Table A. In some embodiments, the one or more genomic variants comprises at least 7 genomic variants from Table A. In some embodiments, one or more genomic variants comprises at least 10 genomic variants from Table A. one or more genomic variants comprises at least 15 genomic variants from Table A. In some embodiments, the one or more genomic variants comprises at least 1, 2, 3, 4, 5, 6, 7, 8, 9, 10, 11, 12, 13, 14, 15, 16, 17, 18, 19, or 20 of the genomic variants from Table A.

In some embodiments, the treatment for an ocular disease disclosed herein comprises a pharmaceutical composition comprising an RBP4 inhibitor or a compound which reduces blood RBP4 concentration in the individual. Further provided herein are methods wherein the pharmaceutical composition comprises a compound having the structure of Formula (I):

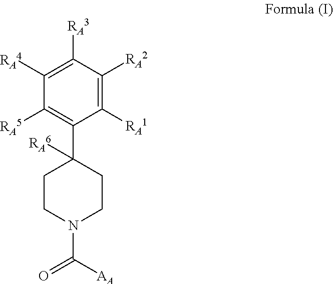

wherein: R_{A}¹, R_{A}², R_{A}³, R_{A}⁴, and R_{A}⁵ are each independently H, halogen, CF₃, or C₁-C₄ alkyl, wherein two or more of R_{A}¹, R_{A}², R_{A}³, R_{A}⁴, and R_{A}⁵ are other than H; R_{A}⁶ is H, OH, or halogen; and A_{A} has the structure:

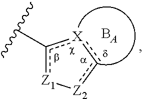

wherein, α, β, χ, and δ are each independently absent or present, and when present each is a bond; X is C or N; Z₁ is N; Z₂ is N or NR_{A}⁹, wherein R_{A}⁹ is H, C₁-C₄ alkyl, or oxetane; B_{A} is a substituted or unsubstituted 5, 6, or 7 membered ring structure; or a pharmaceutically acceptable salt thereof.

Further provided herein are methods wherein the compound has the structure

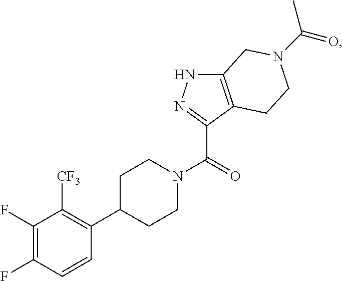 [SMILES: CC(=O)N1CCc2c(C(=O)N3CCC(c4ccc(F)c(F)c4C(F)(F)F)CC3)n[nH]c2C1]

or a pharmaceutically acceptable salt thereof.

Further provided herein are methods wherein the compound has the structure

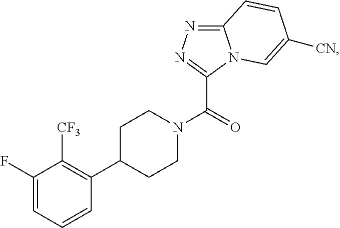 [SMILES: N#Cc1ccc2nnc(C(=O)N3CCC(c4cccc(F)c4C(F)(F)F)CC3)n2c1]

or a pharmaceutically acceptable salt thereof.

Further provided herein are methods wherein the pharmaceutical composition comprises a compound having the structure of Formula (II):

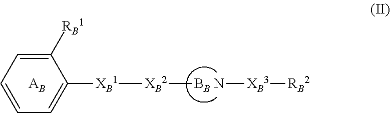

wherein: ring A_{B} is benzene optionally further substituted; R_{B}¹ is an optionally substituted branched C₃-C₆ alkyl group X_{B}¹ is O, S, SO, SO₂, or NH; X_{B}² is a bond or a C₁-C₃ alkylene group; ring B_{B} is azetidine or piperidine; X_{B}³ is CO or SO₂; R_{B}² is an optionally substituted hydrocarbon group, an optionally substituted heterocyclic group, an optionally substituted hydroxy group, an optionally substituted mercapto group, a cyano group, a nitro group, an acyl group, or a halogen atom; or a pharmaceutically acceptable salt thereof.

Further provided herein are methods wherein the compound is 4-(3-(2-tert-butylphenoxy)azetidin-1-yl)-4-oxobutanoic acid, 3-{3-[(2-tert-butyl-4-fluorophenoxy)methyl]azetidin-1-yl}-3-oxopropanoic acid, 2-{[3-(2-tert-butyl-4-chlorophenoxy)azetidin-1-yl]carbonyl}pyridine, 4-[3-(2-tert-butyl-4-chlorophenoxy)azetidin-1-yl]-4-oxobutanoic acid, {3-[(2-tert-butyl-4-chlorophenoxy)methyl]azetidin-1-yl}(oxo)acetic acid, {3-[(2-tert-butylphenoxy)methyl]azetidin-1-yl}(oxo)acetic acid, 3-{3-[(2-tert-butylphenoxy)methyl]azetidin-1-yl}-3-oxopropanoic acid, {4-[(2-tert-butyl-4-chlorophenoxy)methyl]piperidin-1-yl}(oxo)acetic acid, [4-(2-tert-butylphenoxy)piperidin-1-yl](oxo)acetic acid, or {4-[(2-tert-butylphenoxy)methyl]piperidin-1-yl}(oxo)acetic acid, or a pharmaceutically acceptable salt thereof.

Further provided herein are methods wherein the pharmaceutical composition comprises a compound having the structure of Formula (III):

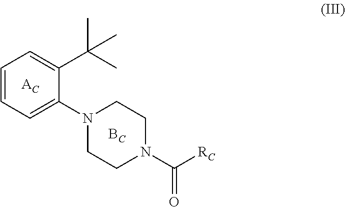

wherein, ring A_{C} is a benzene ring optionally substituted by 1 to 3 substituents selected from the group consisting of (a) a halogen atom, and (b) a C₁₋₆ alkyl group; ring B_{C} is a piperazine ring optionally substituted by 1 to 3 substituents selected from the group consisting of (a) a halogen atom, (b) a C₁-C₆ alkyl group optionally substituted by 1 to 3 halogen atoms, and (c) a C₁-C₆ alkoxy group optionally substituted by 1 to 3 halogen atoms; and R_{C} is (1) an optionally substituted C₁-C₁₀ alkyl group, (2) an optionally substituted C₆-C₁₄ aryl group, (3) an optionally substituted 5- or 6-membered aromatic heterocyclic group, (4) an optionally substituted amino group, (5) an optionally substituted carboxy group, or (6) an optionally substituted carbamoyl group, or a pharmaceutically acceptable salt thereof.

Further provided herein are methods wherein the compound is N-{[4-(2-tert-butylphenyl)piperazin-1-yl]carbonyl}glycine, 3-[4-(2-tert-butylphenyl)piperazin-1-yl]-3-oxopropanoic acid, [4-(2-tert-butyl-4-chlorophenyl)piperazin-1-yl](oxo)acetic acid, 5-{2-[4-(2-tert-butylphenyl)piperazin-1-yl]-2-oxoethyl}imidazolidine-2,4-dione, [(5-{[4-(2-tert-butylphenyl)piperazin-1-yl]carbonyl}isoxazol-3-yl)oxy]acetic acid, or a pharmaceutically acceptable salt thereof.

Further provided herein are methods wherein the pharmaceutical composition comprises a compound having the structure of Formula (IV):

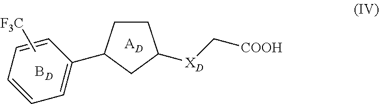

wherein: ring A_{D} is a 5-membered non-aromatic heterocycle optionally further substituted by one oxo group; ring B_{D} is a benzene ring optionally further substituted by 1 to 4 substituents; and X_{D} is 0, CH₂O, OCH₂, CH₂, (CH₂)₂, S, CH₂S, SCH₂, S(O), CH₂S(O), S(O)CH₂, S(O)₂, CH₂S(O)₂, or S(O)₂CH₂; or a pharmaceutically acceptable salt thereof.

Further provided herein are methods wherein the compound is ({(3S)-1-[3,5-bis(trifluoromethyl)phenyl]pyrrolidin-3-yl}oxy)acetic, ({1-[4-chloro-3-(trifluoromethyl)phenyl]pyrrolidin-3-yl}sulfanyl)acetic acid, 3-{(2R,5S)-5-[3,5-bis(trifluoromethyl)phenyl]tetrahydrofuran-2-yl}propanoic acid, or a pharmaceutically acceptable salt thereof.

Further provided herein are methods wherein the pharmaceutical composition comprises a compound having the structure of Formula (V):

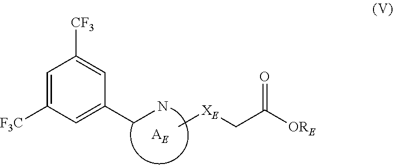

wherein: ring A_{E} is a pyrazole ring, a pyridine ring, an oxazole ring, an imidazole ring, or a pyrimidine ring; X_{E} is S, optionally substituted alkylene, or O; and R_{E} is a hydrogen atom or a C₁. C₆ alkyl group; or a pharmaceutically acceptable salt thereof.

Further provided herein are methods wherein the compound is ((4-(3,5-bis(trifluoromethyl)phenyl)-1,3-oxazol-2-yl)sulfanyl)acetic acid, ethyl ((6-(3,5-bis(trifluoromethyl)phenyl)-pyridin-3-yl)sulfanyl)acetic acid, ((6-(3,5-bis(trifluoromethyl)-phenyl)pyridine-3-yl)sulfanyl)acetic acid, 3-(3-(3,5-bis(trifluoromethyl)phenyl)-1H-pyrazol-1-yl)butanoic acid, or 3-{3-[3,5-bis(trifluoromethyl)phenyl]-1H-pyrazol-1-yl}propanoic acid (also known as STG-001).

In some embodiments, the treatments containing the compound(s) described herein are administered for prophylactic and/or therapeutic treatments. In certain therapeutic applications, the treatments are administered to a patient already suffering from a disease or condition in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, in which the mammal previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Oral doses typically range from about 1.0 mg to about 1000 mg, one to four times, or more, per day. In general, however, doses employed for adult human treatment are typically in the range of 0.01 mg to 5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Once improvement of the patient's conditions has occurred based on clinical evaluation methods describe herein, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED50 with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal. In some embodiments, an RBP4 inhibitor, a pharmaceutically acceptable salt, solvate, polymorph, prodrug, metabolite, N-oxide, stereoisomer, or isomer thereof is administered orally or parenterally to the subject in need thereof. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration. In some embodiments, an RBP4 inhibitor, a pharmaceutically acceptable salt, solvate, polymorph, prodrug, metabolite, N-oxide, stereoisomer, or isomer thereof is administered orally or intravenously to a subject in need thereof. In some embodiments, an RBP4 inhibitor, a pharmaceutically acceptable salt, solvate, polymorph, prodrug, metabolite, N-oxide, stereoisomer, or isomer thereof is administered orally to a subject in need thereof. In some embodiments, an RBP4 inhibitor, a pharmaceutically acceptable salt, solvate, polymorph, prodrug, metabolite, N-oxide, stereoisomer, or isomer thereof is administered intravenously to a subject in need thereof.

In any of the aforementioned aspects are further embodiments comprising single administrations of the effective amount of the compound, including further embodiments in which (i) the compound is administered once a day; or (ii) the compound is administered to the mammal multiple times over the span of one day, e.g., two, three, four or more times daily. In some embodiments, the RBP4 inhibitory compounds described herein are administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the RBP4 inhibitory compounds described herein, or a pharmaceutically acceptable salt, solvate, polymorph, prodrug, metabolite, N-oxide, stereoisomer, or isomer thereof, are administered daily.

In any of the aforementioned aspects are further embodiments comprising multiple administrations of the effective amount of the compound, including further embodiments in which (i) the compound is administered continuously or intermittently: as in a single dose; (ii) the time between multiple administrations is every 3 hours; (iii) the time between multiple administrations is every 6 hours; (iv) the compound is administered to the mammal every 8 hours; (v) the compound is administered to the mammal every 12 hours; or (vi) the compound is administered to the mammal every 24 hours.

In certain embodiments wherein a patient's status does improve, the dose of drug being administered is temporarily reduced or temporarily suspended for a certain length of time (e.g., a “drug holiday”). In specific embodiments, the length of the drug holiday is 2 days to 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 7 days. In one embodiment, the length of the drug holiday is 7 days. In one embodiment, the length of the drug holiday is 14 days. In one embodiment, the length of the drug holiday is 28 days.

### Retinol Binding Protein 4

In an aspect provided herein is a method of diagnosing or assessing the risk of developing Age-Related Macular Degeneration (AMD) in an individual in need thereof comprising determining by an assay a level of retinol binding protein 4 (RBP4) in a sample from the individual. In some embodiments, the diagnosing or risk assessment step is accompanied by a treatment step, such as the administration of a therapy to reduce the level of RBP4 in the individual, thereby treating or preventing the AMD in the individual. In some embodiments, the therapy is administered if the level of RBP4 in the sample is above a threshold value (e.g. 25 pig/mL). In some embodiments, the individual is diagnosed or assessed to be at risk of developing AMD if the level of RBP4 in the sample is above a threshold value (e.g. 25 μg/mL).

In another aspect provided herein is a method for assessing the likelihood of age-related macular degeneration in an individual comprising determining by an assay a level of retinol binding protein 4 (RBP4) in a sample from the individual is above a threshold value of RBP4 and assessing the likelihood of developing macular degeneration based on the level of RBP4. In some embodiments, the method also comprises determining the age and/or medical history of the individual. In some embodiments, thus, assessing the likelihood of developing macular degeneration is based on the level of RBP4, age, and medical history of the individual.

In another aspect provided herein is a method for assessing the severity of age-related macular degeneration in an individual comprising determining by an assay a level of retinol binding protein 4 (RBP4) in a sample from the individual is above a threshold value of RBP4 and assessing the severity of the macular degeneration based on the level of RBP4. In some embodiments, the method also comprises determining the age and/or medical history of the individual. In some embodiments, thus, assessing the severity of macular degeneration is based on the level of RBP4, age, and medical history of the individual.

In another aspect provided herein is a method for assessing a diagnosis of age-related macular degeneration in an individual comprising determining by an assay a level of retinol binding protein 4 (RBP4) in a sample from the individual is above a threshold value of RBP4 and assessing the diagnosis of macular degeneration based on the level of RBP4. In some embodiments, the method also comprises determining the age and/or medical history of the individual. In some embodiments, thus, assessing diagnosis of macular degeneration is based on the level of RBP4, age, and medical history of the individual.

In another aspect provided herein is a method for assessing a dosing regimen in an individual with age-related macular degeneration comprising determining by an assay a level of retinol binding protein 4 (RBP4) in a sample from the individual is above a threshold value of RBP4 and assessing the diagnosis of macular degeneration based on the level of RBP4. In some embodiments, the method also comprises determining the age and/or medical history of the individual. In some embodiments, thus, assessing a dosing regimen in an individual with macular degeneration is based on the level of RBP4, age, and medical history of the individual.

In some embodiments, the threshold level of RBP4 is at least 25 μg/mL. In some embodiments, the threshold level of RBP4 is at least 30 μg/mL. In some embodiments, the threshold level of RBP4 is at least 35 μg/mL. In some embodiments, the threshold value is about 25 μg/mL to about 100 μg/mL. In some embodiments, the threshold value is about 25 μg/mL to about 30 μg/mL, about 25 μg/mL to about 35 μg/mL, about 25 μg/mL to about 40 μg/mL, about 25 μg/mL to about 50 g/mL, about g/mL to about g/mL, about g/mL to about g/mL, about 25 μg/mL to about 80 g/mL, about 25 μg/mL to about 90 μg/mL, about 25 μg/mL to about 100 μg/mL, about 30 μg/mL to about 35 μg/mL, about 30 μg/mL to about 40 μg/mL, about 30 μg/mL to about 50 μg/mL, about 30 g/mL to about 60 μg/mL, about 30 μg/mL to about 70 μg/mL, about 30 μg/mL to about 80 μg/mL, about 30 μg/mL to about 90 μg/mL, about 30 μg/mL to about 100 μg/mL, about 35 μg/mL to about g/mL, about 35 μg/mL to about 50 μg/mL, about 35 μg/mL to about 60 μg/mL, about 35 μg/mL to about 70 μg/mL, about 35 μg/mL to about 80 μg/mL, about 35 μg/mL to about 90 μg/mL, or about g/mL to about 100 μg/mL. In some embodiments, the threshold value is about 25 μg/mL, about g/mL, or about 35 μg/mL. In some embodiments, the threshold value is at least about 25 μg/mL, about 30 μg/mL, or about 35 μg/mL. In some embodiments, the threshold value is at most about 30 g/mL, about 35 μg/mL, about 40 μg/mL, about 50 μg/mL, about 60 μg/mL, about 70 μg/mL, about 80 μg/mL, about 90 μg/mL, or about 100 μg/mL.

In some embodiments, the threshold value may vary based on various characteristics of the individual. Non-limiting examples of such characteristics comprises demographic data, gender, age, medical history, previous diagnosis with an eye condition, body-mass index (BMI), height, weight, a systemic condition including obesity, hyperlipidemia, hyperglycemia, hypertension, diabetes, mellitus, cardiovascular disease, or non-alcoholic fatty liver diseases.

In some embodiments, a diagnosis of AMD is made if the level of RBP4 is above the threshold value. In some embodiments, a risk score is calculated if the level of RBP4 is above the threshold value.

In some embodiments, a therapy is administered if the level of RBP4 is above the threshold value. In some embodiments, the therapy is administered to reduce the level of RBP4. In some embodiments, the therapy comprises administering a pharmaceutical composition to the individual. In some embodiments, the therapy comprises administering a pharmaceutical compound to the individual. In some embodiments, the pharmaceutical composition comprises an RBP4 inhibitor. The RBP4 inhibitor may be any of the RBP4 inhibitors provided herein.

In some embodiments, an additional step may be taken or recommended if the level of RBP4 is below the threshold value. Such a reading below the threshold value may still indicate that the individual bears some risk of developing AMID in the future and thus future monitoring may be recommended, or additional tests may be needed to confirm or complete a diagnosis of AMD.

Additionally, in some embodiments, the method comprises assessing the likelihood of developing AMID based on the level of RBP4. In some embodiments, assessing the likelihood of developing AMD comprises generating a risk score. In some embodiments, if the risk score is above a threshold value, a treatment of AMD is administered (e.g. treatment with an RBP4 inhibitor). In some embodiments, the risk score reflects the probability that the individual will develop AMD at a point in the future based off the measurements provided herein.

In some embodiments, the method comprises providing a recommendation to reassess the individual for AMD after a period of time (e.g., when a risk score for development of AMD has been calculated or when the level of RBP4 is below the threshold value but there are other indications that AMD may develop). In some embodiments, if the level or the risk score is below the threshold value, the method comprises providing a recommendation to reassess the individual for AMD after a period of time. The period of time may be any suitable period of time. In some embodiments, the period of time is about 1 month to about 24 months. In some embodiments, the period of time is about 1 month to about 3 months, about 1 month to about 6 months, about 1 month to about 9 months, about 1 month to about 12 months, about 1 month to about 18 months, about 1 month to about 24 months, about 3 months to about 6 months, about 3 months to about 9 months, about 3 months to about 12 months, about 3 months to about 18 months, about 3 months to about 24 months, about 6 months to about 9 months, about 6 months to about 12 months, about 6 months to about 18 months, about 6 months to about 24 months, about 9 months to about 12 months, about 9 months to about 18 months, about 9 months to about 24 months, about 12 months to about 18 months, about 12 months to about 24 months, or about 18 months to about 24 months. In some embodiments, the period of time is about 1 month, about 3 months, about 6 months, about 9 months, about 12 months, about 18 months, or about 24 months. In some embodiments, the period of time is at least about 1 month, about 3 months, about 6 months, about 9 months, about 12 months, or about 18 months. In some embodiments, the period of time is at most about 3 months, about 6 months, about 9 months, about 12 months, about 18 months, or about 24 months.

The recommendation to reassess comprises the performing an additional test after the period of time, including without limitation reassessing the level of RBP4 in the individual or performing any of the other tests or assessments for AMD provided herein, such as a follow up with a physician for a physical examination of the eye.

In some embodiments, the level of RBP4 in the individual is assessed by an assay. Any suitable assay for measuring the level of RBP4 can be employed (e.g. antibody assay, mass spectrometry based assay (e.g. LC/MS), liquid chromatography assay (e.g. HPLC, UPLC), etc.). In some embodiments, the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. In some embodiments, the assay comprises an antibody assay. In some embodiments, the antibody assay comprises an enzyme-linked immunosorbent assay (ELISA).

In some embodiments, the sample comprises a blood sample from the individual. The blood sample may be further processed, such as to remove impurities or to leave behind the serum and/or plasma. In some embodiments, the sample is a plasma or serum sample. In some embodiments, the sample is a plasma sample. In some embodiments, the sample is a serum sample. In some embodiments, the level is measured from plasma or serum derived from the blood sample. In some embodiments, the level is measured from plasma derived from the blood sample. In some embodiments, the level is measured from serum derived from the blood sample.

In some embodiments, the determination of the diagnosis of AMD, the risk assessment of developing AMD, and/or the decision to administer a therapy is made based on additional considerations in addition to the level of RBP4. In some embodiments, the determination is based on the age and/or medical history of the individual. In some embodiments, the determination is based at least partially on the age of the individual. In some embodiments, the determination is based at least partially on the medical history of the individual. In some embodiments, the medical history of the individual can include information such as previous diagnosis with an eye condition, body-mass index (BMI), height, weight, systemic condition including obesity, hyperlipidemia, hyperglycemia, hypertension, diabetes mellitus, cardiovascular disease, or non-alcoholic fatty liver diseases. In some embodiments, the determination is based at least partially on a level of vitamin A in the individual (e.g., in a blood sample of the individual).

Additionally, the methods provided herein may further comprise any of the additional tests or diagnostics for assessing the presence of AMD in an individual provided herein, including measurement of the presence or absence of any of the genomic variants provided herein or any other measurement which can be used to diagnose AMD. In some embodiments, the method further comprises performing at least one of color fundus photography, fundus autofluorescence, spectral-domain optical coherence tomography, or microperimetry.

In some embodiments, the method further comprises classifying a progression of age-related macular degeneration. The classification can be based on the level of RBP4, measured as provided herein, or in conjunction with other methods provided herein, such as by classification using Age-Related Eye Disease Study (AREDS) classifications.

### Vitamin A Levels Predictive of AMD

In an aspect provided herein is a method of diagnosing or assessing the risk of developing Age-Related Macular Degeneration (AMD) in an individual in need thereof comprising determining by an assay a level of vitamin A in a sample from the individual. In some embodiments, the diagnosing or risk assessment step is accompanied by a treatment step, such as the administration of a therapy to reduce the level of vitamin A or RBP4 in the individual, thereby treating or preventing the AMD in the individual. In some embodiments, the therapy is administered if the level of vitamin A in the sample is above a threshold value (e.g. 150 ng/mL). In some embodiments, the individual is diagnosed or assessed to be at risk of developing AMD if the level of vitamin A in the sample is above a threshold value (e.g. 150 ng/mL).

In another aspect provided herein is a method for assessing the likelihood of age-related macular degeneration in an individual comprising determining by an assay a level of vitamin A in a sample from the individual is above a threshold value of vitamin A and assessing the likelihood of developing macular degeneration based on the level of vitamin A. In some embodiments, the method also comprises determining the age and/or medical history of the individual. In some embodiments, thus, assessing the likelihood of developing macular degeneration is based on the level of vitamin A, age, and medical history of the individual.

In another aspect provided herein is a method for assessing the severity of age-related macular degeneration in an individual comprising determining by an assay a level vitamin A in a sample from the individual is above a threshold value of vitamin A and assessing the severity of the macular degeneration based on the level of vitamin A. In some embodiments, the method also comprises determining the age and/or medical history of the individual. In some embodiments, thus, assessing the severity of macular degeneration is based on the level of vitamin A, age, and medical history of the individual.

In another aspect provided herein is a method for assessing a diagnosis of age-related macular degeneration in an individual comprising determining by an assay a level vitamin A in a sample from the individual is above a threshold value of vitamin A and assessing the diagnosis of macular degeneration based on the level of vitamin A. In some embodiments, the method also comprises determining the age and/or medical history of the individual. In some embodiments, thus, assessing diagnosis of macular degeneration is based on the level of vitamin A, age, and medical history of the individual.

In another aspect provided herein is a method for assessing a dosing regimen in an individual with age-related macular degeneration comprising determining by an assay a level of vitamin Ain a sample from the individual is above a threshold value of vitamin A and assessing the diagnosis of macular degeneration based on the level of vitamin A. In some embodiments, the method also comprises determining the age and/or medical history of the individual. In some embodiments, thus, assessing a dosing regimen in an individual with macular degeneration is based on the level of vitamin A, age, and medical history of the individual.

In some embodiments, the threshold level of Vitamin A is at least about 150 ng/mL. In some embodiments, the threshold level of Vitamin A is at least about 175 ng/mL. In some embodiments, the threshold level of Vitamin A is at least about 200 ng/mL. In some embodiments, the threshold level of Vitamin A is at least about 220 ng/mL. In some embodiments, the threshold level of Vitamin A is at least about 221 ng/mL. In some embodiments, the threshold level of Vitamin A is at least about 222 ng/mL. In some embodiments, the threshold level of Vitamin A is at least about 223 ng/mL. In some embodiments, the threshold level of Vitamin A is at least about 224 ng/mL. In some embodiments, the threshold level of Vitamin A is at least about 225 ng/mL. In some embodiments, the threshold level of Vitamin A is at least about 250 ng/mL. In some embodiments, the threshold level of Vitamin A is at least about 300 ng/mL. In some embodiments, the threshold level of Vitamin A is at least about 350 ng/mL. In some embodiments, the threshold level of Vitamin A is at least about 390 ng/mL. In some embodiments, the threshold level of Vitamin A is at least about 391 ng/mL. In some embodiments, the threshold level of Vitamin A is at least about 392 ng/mL. In some embodiments, the threshold level of Vitamin A is at least about 393 ng/mL. In some embodiments, the threshold level of Vitamin A is at least about 394 ng/mL. In some embodiments, the threshold level of Vitamin A is at least about 395 ng/mL. In some embodiments, the threshold level of vitamin A is about 150 ng/mL to about 500 ng/mL. In some embodiments, the threshold level of vitamin A is about 150 ng/mL to about 175 ng/mL, about 150 ng/mL to about 200 ng/mL, about 150 ng/mL to about 225 ng/mL, about 150 ng/mL to about 250 ng/mL, about 150 ng/mL to about 300 ng/mL, about 150 ng/mL to about 350 ng/mL, about 150 ng/mL to about 400 ng/mL, about 150 ng/mL to about 450 ng/mL, about 150 ng/mL to about 500 ng/mL, about 175 ng/mL to about 200 ng/mL, about 175 ng/mL to about 225 ng/mL, about 175 ng/mL to about 250 ng/mL, about 175 ng/mL to about 300 ng/mL, about 175 ng/mL to about 350 ng/mL, about 175 ng/mL to about 400 ng/mL, about 175 ng/mL to about 450 ng/mL, about 175 ng/mL to about 500 ng/mL, about 200 ng/mL to about 225 ng/mL, about 200 ng/mL to about 250 ng/mL, about 200 ng/mL to about 300 ng/mL, about 200 ng/mL to about 350 ng/mL, about 200 ng/mL to about 400 ng/mL, about 200 ng/mL to about 450 ng/mL, about 200 ng/mL to about 500 ng/mL, about 225 ng/mL to about 250 ng/mL, about 225 ng/mL to about 300 ng/mL, about 225 ng/mL to about 350 ng/mL, about 225 ng/mL to about 400 ng/mL, about 225 ng/mL to about 450 ng/mL, about 225 ng/mL to about 500 ng/mL, about 250 ng/mL to about 300 ng/mL, about 250 ng/mL to about 350 ng/mL, about 250 ng/mL to about 400 ng/mL, about 250 ng/mL to about 450 ng/mL, about 250 ng/mL to about 500 ng/mL, about 300 ng/mL to about 350 ng/mL, about 300 ng/mL to about 400 ng/mL, about 300 ng/mL to about 450 ng/mL, about 300 ng/mL to about 500 ng/mL, about 350 ng/mL to about 400 ng/mL, about 350 ng/mL to about 450 ng/mL, about 350 ng/mL to about 500 ng/mL, about 400 ng/mL to about 450 ng/mL, about 400 ng/mL to about 500 ng/mL, or about 450 ng/mL to about 500 ng/mL. In some embodiments, the threshold level of vitamin A is about 150 ng/mL, about 175 ng/mL, about 200 ng/mL, about 225 ng/mL, about 250 ng/mL, about 300 ng/mL, about 350 ng/mL, about 400 ng/mL, about 450 ng/mL, or about 500 ng/mL. In some embodiments, the threshold level of vitamin A is at least about 150 ng/mL, about 175 ng/mL, about 200 ng/mL, about 225 ng/mL, about 250 ng/mL, about 300 ng/mL, about 350 ng/mL, about 400 ng/mL, or about 450 ng/mL. In some embodiments, the threshold level of vitamin A is at most about 175 ng/mL, about 200 ng/mL, about 225 ng/mL, about 250 ng/mL, about 300 ng/mL, about 350 ng/mL, about 400 ng/mL, about 450 ng/mL, or about 500 ng/mL.

In some embodiments, the threshold value may vary based on various characteristics of the individual. Non-limiting examples of such characteristics comprises demographic data, gender, age, medical history, previous diagnosis with an eye condition, body-mass index (BMI), height, weight, a systemic condition including obesity, hyperlipidemia, hyperglycemia, hypertension, diabetes, mellitus, cardiovascular disease, or non-alcoholic fatty liver diseases.

In some embodiments, the threshold level of vitamin A depends on the age of the individual. In some embodiments, the threshold level of vitamin A is lesser for an adolescent (e.g., an individual with an age of 12-17) than for an adult (e.g., an age of 18 or older). In some embodiments, the threshold level of vitamin A for an adolescent is at least about 150 ng/mL, at least about 175 ng/mL, at least about 200 ng/mL, at least about 210 ng/mL, at least about 220 ng/mL, at least about 221 ng/mL, at least about 222 ng/mL, at least about 223 ng/mL, at least about 224 ng/mL, at least about 225 ng/mL, at least about 230 ng/mL, or at least about 250 ng/mL. In some embodiments, the threshold for an adult is at least about 300 ng/mL, at least about 325 ng/mL, at least about 350 ng/mL, at least about 375 ng/mL, at least about 380 ng/mL, at least about 385 ng/mL, at least about 390 ng/mL, at least about 391 ng/mL, at least about 392 ng/mL, at least about 393 ng/mL, at least about 394 ng/mL, at least about 395 ng/mL, or at least about 400 ng/mL.

In some embodiments, a diagnosis of AMD is made if the level of vitamin A is above the threshold value. In some embodiments, a risk score is calculated if the level of vitamin A is above the threshold value.

In some embodiments, a therapy is administered if the level of vitamin A is above the threshold value. In some embodiments, the therapy is administered to reduce the level of vitamin A. In some embodiments, the therapy comprises administering a pharmaceutical composition to the individual. In some embodiments, the therapy comprises administering a pharmaceutical compound to the individual. In some embodiments, the pharmaceutical composition comprises an RBP4 inhibitor. The RBP4 inhibitor may be any of the RBP4 inhibitors provided herein.

In some embodiments, an additional step may be taken or recommended if the level of vitamin A is below the threshold value. Such a reading below the threshold value may still indicate that the individual bears some risk of developing AMD in the future and thus future monitoring may be recommended, or additional tests may be needed to confirm or complete a diagnosis of AMD.

Additionally, in some embodiments, the method comprises assessing the likelihood of developing AMD based on the level of vitamin A. In some embodiments, assessing the likelihood of developing AMD comprises generating a risk score. In some embodiments, if the risk score is above a threshold value, a treatment of AMD is administered (e.g. treatment with an RBP4 inhibitor). In some embodiments, the risk score reflects the probability that the individual will develop AMD at a point in the future based off the measurements provided herein.

In some embodiments, the method comprises providing a recommendation to reassess the individual for AMD after a period of time (e.g., when a risk score for development of AMD has been calculated or when the level of vitamin A is below the threshold value but there are other indications that AMD may develop). In some embodiments, if the level or the risk score is below the threshold value, the method comprises providing a recommendation to reassess the individual for AMD after a period of time. The period of time may be any suitable period of time. In some embodiments, the period of time is about 1 month to about 24 months. In some embodiments, the period of time is about 1 month to about 3 months, about 1 month to about 6 months, about 1 month to about 9 months, about 1 month to about 12 months, about 1 month to about 18 months, about 1 month to about 24 months, about 3 months to about 6 months, about 3 months to about 9 months, about 3 months to about 12 months, about 3 months to about 18 months, about 3 months to about 24 months, about 6 months to about 9 months, about 6 months to about 12 months, about 6 months to about 18 months, about 6 months to about 24 months, about 9 months to about 12 months, about 9 months to about 18 months, about 9 months to about 24 months, about 12 months to about 18 months, about 12 months to about 24 months, or about 18 months to about 24 months. In some embodiments, the period of time is about 1 month, about 3 months, about 6 months, about 9 months, about 12 months, about 18 months, or about 24 months. In some embodiments, the period of time is at least about 1 month, about 3 months, about 6 months, about 9 months, about 12 months, or about 18 months. In some embodiments, the period of time is at most about 3 months, about 6 months, about 9 months, about 12 months, about 18 months, or about 24 months.

The recommendation to reassess comprises the performing an additional test after the period of time, including without limitation reassessing the level of vitamin A in the individual or performing any of the other tests or assessments for AMD provided herein, such as a follow up with a physician for a physical examination of the eye.

In some embodiments, the level of vitamin A in the individual is assessed by an assay. Any suitable assay for measuring the level of vitamin A can be employed (e.g., antibody assay, mass spectrometry based assay (e.g., LC/MS), liquid chromatography assay (e.g., HPLC, UPLC), etc.). In some embodiments, the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. In some embodiments, the assay comprises an antibody assay. In some embodiments, the antibody assay comprises an enzyme-linked immunosorbent assay (ELISA). In some embodiments, the assay is a chromatographic assay. In some embodiments, the assay comprises high performance liquid chromatography (HPLC), ultra-performance liquid chromatography (UPLC), or liquid chromatography/mass spectrometry (LC-MS).

In some embodiments, the sample comprises a blood sample from the individual. The blood sample may be further processed, such as to remove impurities or to leave behind the serum and/or plasma. In some embodiments, the sample is a plasma or serum sample. In some embodiments, the sample is a plasma sample. In some embodiments, the sample is a serum sample. In some embodiments, the level is measured from plasma or serum derived from the blood sample. In some embodiments, the level is measured from plasma derived from the blood sample. In some embodiments, the level is measured from serum derived from the blood sample.

In some embodiments, the determination of the diagnosis of AMD, the risk assessment of developing AMD, and/or the decision to administer a therapy is made based on additional considerations in addition to the level of vitamin A. In some embodiments, the determination is based on the age and/or medical history of the individual. In some embodiments, the determination is based at least partially on the age of the individual. In some embodiments, the determination is based at least partially on the medical history of the individual. In some embodiments, the medical history of the individual can include information such as previous diagnosis with an eye condition, body-mass index (BMI), height, weight, systemic condition including obesity, hyperlipidemia, hyperglycemia, hypertension, diabetes mellitus, cardiovascular disease, or non-alcoholic fatty liver diseases. In some embodiments, the determination is based at least partially on a level of RBP4 in the individual (e.g., in a blood sample of the individual).

Additionally, the methods provided herein may further comprise any of the additional tests or diagnostics for assessing the presence of AMD in an individual provided herein, including measurement of the presence or absence of any of the genomic variants provided herein or any other measurement which can be used to diagnose AMD. In some embodiments, the method further comprises performing at least one of color fundus photography, fundus autofluorescence, spectral-domain optical coherence tomography, or microperimetry.

In some embodiments, the method further comprises classifying a progression of age-related macular degeneration. The classification can be based on the level of vitamin A, measured as provided herein, or in conjunction with other methods provided herein, such as by classification using Age-Related Eye Disease Study (AREDS) classifications.

### Genomic Variants Predictive of AMD

Also provided herein are genomic variants indicative of an individual's propensity to develop AMD. In some embodiments, the genomic variants comprise single nucleotide polymorphisms (SNPs) in a gene encoding ATP-binding cassette, subfamily A, member 4 (ABCA4) in the individual. ABCA4 is a protein encoded by the ABCA4 gene in humans and other eukaryotes. The ABCA4 protein is expressed almost exclusively in the retina and is implicated in Stargardt and other eye diseases, including but not limited to fundus flavimaculatus, cone-rod dystrophy, retinitis pigmentosa, and age-related macular degeneration. Diminished ABCA4 activity is linked with excessive accumulation of toxic retinoids and lipofuscin. Such mutations in some instances are detected by sequencing a subject's DNA or RNA. In some embodiments, the genomic variants provided herein are predictive of a subject's likelihood of developing AMD, either alone or in combination with other factors. The genomic variants herein comprises SNPs which are missense SNPs, intronic SNPs, synonymous SNPs, or any other type of SNP. Individuals comprises one or more of the SNPs provided herein, either on the same allele or on different alleles. Examples of such genomic variants and SNPs can be found in Table A below.

TABLE A
Genomic Variants

| Variant | | | Protein | |
|---|---|---|---|---|
| Number | Variant | Mutation | Change | Type |
| 1 | rs3747961 | c.*372 = | NA | 3 Prime UTR variant |
| 2 | rs6666652 | c.6764G > T | p.S2255I | Missense |
| 3 | rs1800717 | NA | NA | Intronic |
| 4 | rs763108716 | c.6579C > T | p.F2193 = | Synonymous |
| 5 | rs185601596 | NA | NA | Intronic |
| 6 | rs17110761 | NA | NA | Intronic |
| 7 | rs61748519 | c.6255C > T | p.L2085 = | Synonymous |
| 8 | rs1801359 | c.6249C > T | p.I2083 = | Synonymous |
| 9 | rs145766145 | NA | NA | Intronic |
| 10 | rs76258939 | c.3626T > C | p.M1209T | Missense |
| 11 | rs200551567 | c.2841C > T | p.D947 = | Synonymous |
| 12 | rs754765 164 | NA | NA | Intronic splice |
| | | | | acceptor variant |
| 13 | rs201602424 | c.1614C > T | p.A538 = | Synonymous |
| 14 | rs564661476 | NA | NA | Intronic insertion |
| 15 | rs4147831 | c.1269C > T | p.H423 = | Synonymous |
| 16 | rs6657239 | c.635G > A | p.R212H | Missense |
| 17 | rs2297632 | NA | NA | Intronic |
| 18 | rs1801555 | c.6285T > C | p.D2095 = | Synonymous |
| 19 | rs1762114 | c.6069T > C | p.I2023 = | Synonymous |
| 20 | rs4147863% | NA | NA | Intronic |
| 21 | rs2275029% | c.5844A > G | p.P1948 = | Synonymous |
| 22 | rs1800739% | NA | NA | Intronic |
| 23 | rs4147857% | c.5814A > G | p.L1938 = | Synonymous |
| 24 | rs4147856% | NA | NA | Intronic |
| 25 | rs1801574% | c.5682G > C | p.L1894 = | Synonymous |
| 26 | rs55860151 | NA | NA | Intronic |
| 27 | rs1800549 | c.4283C > T | p.T1428M | Missense |
| 28 | rs3112831 | c.1268A > C | p.H423R | Synonymous |
| 29 | rs4147830 | NA | NA | Intronic |
| 30 | rs2297634 | NA | NA | Intronic |
| 31 | rs4847281 | c.141A > G | p.P47 = | Synonymous |

In an aspect provided herein is a method of assessing whether an individual has AMID, is likely to develop AMID, and, in some instances, methods of treating or preventing said AMID. In some embodiments, the method comprises determining by an assay the presence or absence of one or more genomic variants. In some embodiments, the one or more genomic variants comprises at least one of rs4147863, rs2275029, rs11800739, rs4147857, rs4147856, rs1801555, or rs1801574. In some embodiments, the method comprises calculating a risk score for age-related macular degeneration using the presence or absence of the one or more genomic variants. In some embodiments, the method comprises diagnosing age-related macular degeneration using the presence or absence of the one or more genomic variants. In some embodiments, the method further comprises administering a therapy to treat age-related macular degeneration in the individual. In some embodiments, the method further comprises administering a therapy to prevent age-related macular degeneration in the individual.

In some embodiments, the method comprises determining by an assay the presence or absence of one or more genomic variants. In some embodiments, the one or more genomic variants comprises at least one of rs4147863, rs2275029, rs1800739, rs4147857, rs4147856, rs1801555, or rs1801574. In some embodiments, the one or more genomic variants comprises at least two of rs4147863, rs2275029, rs1800739, rs4147857, rs4147856, rs1801555, or rs1801574. In some embodiments, the one or more genomic variants comprises at least three of rs4147863, rs2275029, rs1800739, rs4147857, rs4147856, rs1801555, or rs1801574. In some embodiments, the one or more genomic variants comprises at least four of rs4147863, rs2275029, rs1800739, rs4147857, rs4147856, rs1801555, or rs1801574. In some embodiments, the one or more genomic variants comprises at least five of rs4147863, rs2275029, rs1800739, rs4147857, rs4147856, rs1801555, or rs1801574. In some embodiments, the one or more genomic variants comprises at least six of rs4147863, rs2275029, rs1800739, rs4147857, rs4147856, rs1801555, or rs1801574. In some embodiments, the one or more genomic variants comprises each of rs4147863, rs2275029, rs1800739, rs4147857, rs4147856, rs1801555, or rs1801574.

In some embodiments, the one or more genomic variants comprises rs4147863. In some embodiments, the one or more genomic variants comprises rs2275029. In some embodiments, the one or more genomic variants comprises rs1800739. In some embodiments, the one or more genomic variants comprises rs4147857. In some embodiments, the one or more genomic variants comprises rs4147856. In some embodiments, the one or more genomic variants comprises rs1801555. In some embodiments, the one or more genomic variants comprises rs1801574.

In some embodiments, the one or more genomic variants comprises at least one of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555, rs1762114, rs55860151, rs1800549, rs3112831, rs4147830, rs2297634, or rs4847281. In some embodiments, the one or more genomic variants comprises at least five of rs3747961, rs6666652, rs1800717, rs763108716, rs185601596, rs17110761, rs61748519, rs11801359, rs145766145, rs76258939, rs200551567, rs754765164, rs201602424, rs564661476, rs4147831, rs6657239, rs2297632, rs1801555, rs1762114, rs55860151, rs1800549, rs3112831, rs4147830, rs2297634, or rs4847281. In some embodiments, the one or more genomic variants comprises at least 1, 2, 3, 4, 5, 6, 7, 8, 9, 10, 11, 12, 13, 14, 15, 16, 17, 18, 19, or 20 of the genomic variants.

In some embodiments, the one or more genomic variants comprises at least 1 genomic variant from Table A. In some embodiments, the one or more genomic variants comprises at least 5 genomic variants from Table A. In some embodiments, the one or more genomic variants comprises at least 7 genomic variants from Table A. In some embodiments, one or more genomic variants comprises at least 10 genomic variants from Table A. one or more genomic variants comprises at least 15 genomic variants from Table A. In some embodiments, the one or more genomic variants comprises at least 1, 2, 3, 4, 5, 6, 7, 8, 9, 10, 11, 12, 13, 14, 15, 16, 17, 18, 19, or 20 of the genomic variants from Table A.

In some embodiments, detecting the presence or absence of the one or more genomic variants further comprises determining the allele frequency of the one or more genomic variants in the individual. In some embodiments, the one or more genomic variants are present on a single copy of the gene in the individual. In some embodiments, the one or more genomic variants are present on a plurality of copies of the gene in the individual.

In some embodiments determining the presence or absence of one or more genomic variants comprises performing an assay on genetic material form the individual. In some cases, the genetic material is obtained from blood, serum, plasma, sweat, hair, tears, urine, and other techniques known by one of skill in the art. In some embodiments, the genetic material is obtained from blood, serum, or plasma of the individual.

In some embodiments, methods of detecting a presence, absence, or level of a genomic variant in the sample obtained from the individual involve detecting a nucleic acid sequence. In some cases, the nucleic acid sequence comprises deoxyribonucleic acid (DNA), such as in the case of detecting complementary DNA (cDNA) of an mRNA transcript. In some instances, the nucleic acid sequence comprises a denatured DNA molecule or fragment thereof. In some instances, the nucleic acid sequence comprises DNA selected from: genomic DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises ribonucleic acid (RNA). In some instances, the nucleic acid sequence comprises fragmented RNA. In some instances, the nucleic acid sequence comprises partially degraded RNA.

Disclosed herein, in some embodiments, the genomic variant is detected by subjecting a sample obtained from the subject to a nucleic acid-based detection assay. In some instances, the nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytochemistry, microarray, or sequencing. In some embodiments, the sequencing technique comprises next generation sequencing. In some embodiments, the methods involve a hybridization assay such as fluorogenic qPCR (e.g., TaqMan™, SYBR green, SYBR green I, SYBR green II, SYBR gold, ethidium bromide, methylene blue, Pyronin Y, DAPI, acridine orange, Blue View or phycoerythrin), which involves a nucleic acid amplification reaction with a specific primer pair, and hybridization of the amplified nucleic acid probes comprising a detectable moiety or molecule that is specific to a target nucleic acid sequence. In some instances, a number of amplification cycles for detecting a target nucleic acid in a qPCR assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles. In some instances, hybridization may occur at standard hybridization temperatures, e.g., about 35° C. to about 65° C. in a standard PCR buffer.

An additional exemplary nucleic acid-based detection assay comprises the use of nucleic acid probes conjugated or otherwise immobilized on a bead, multi-well plate, or other substrate, wherein the nucleic acid probes are configured to hybridize with a target nucleic acid sequence. In some instances, the nucleic acid probe is specific to one or more gene products described herein (of the PRS). In some instances, the nucleic acid probe specific to a biomarker comprises a nucleic acid probe sequence sufficiently complementary to the polynucleotide sequence of the biomarker. In some instances, the biomarker comprises a transcribed polynucleotide sequence (e.g., RNA, cDNA). In some embodiments, the nucleic acid probe can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the target nucleic acid sequence. In some embodiments, the target nucleic acid sequence is immobilized on a solid surface and contacted with a probe, for example by running the isolated target nucleic acid sequence on an agarose gel and transferring the target nucleic acid sequence from the gel to a membrane, such as nitrocellulose. In some embodiments, the probe(s) are immobilized on a solid surface, for example, in an Affymetrix gene chip array, and the probe(s) are contacted with the target nucleic acid sequence.

In some embodiments, the term “probe” with regards to nucleic acids, refers to any nucleic acid molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some embodiments, detecting the one or more genomic variants comprises sequencing genetic material obtained from a sample from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some embodiments, a therapy is administered if the genomic variants provided herein are identified in the individual. In some embodiments, the therapy is administered to reduce the level of RBP4 in the individual. In some embodiments, the therapy comprises administering a pharmaceutical composition to the individual. In some embodiments, the therapy comprises administering a pharmaceutical compound to the individual. In some embodiments, the pharmaceutical composition comprises an RBP4 inhibitor. The RBP4 inhibitor may be any of the RBP4 inhibitors provided herein.

Additionally, the methods provided herein may further comprise any of the additional tests or diagnostics for assessing the presence of AMD in an individual provided herein, including measurements of the level of RBP4 and comparison to a threshold value as provided herein, measurements of the level of vitamin A and comparison to a threshold value as provided herein, or any other measurement which can be used to diagnose AMD. In some embodiments, the method further comprises performing at least one of color fundus photography, fundus autofluorescence, spectral-domain optical coherence tomography, or microperimetry.

In some embodiments, the method also comprises determining the age and medical history of the individual. In some embodiments, thus, assessing the likelihood of developing macular degeneration is based on presence of the one or more genomic variants, age, and medical history of the individual.

In some embodiments, the determination of the diagnosis of AMD, the risk assessment of developing AMD, or the decision to treat is made based on additional considerations in addition to the presence or absence of the one or more genomic variants. In some embodiments, the determination is based on the age and/or medical history of the individual. In some embodiments, the determination is based at least partially on the age of the individual. In some embodiments, the determination is based at least partially on the medical history of the individual. In some embodiments, the medical history of the individual can include information such as previous diagnosis with an eye condition, body-mass index (BMI), height, weight, systemic condition including obesity, hyperlipidemia, hyperglycemia, hypertension, diabetes mellitus, cardiovascular disease, or non-alcoholic fatty liver diseases.

In some embodiments, identification of the one or more genomic variants provided herein in an individual does not necessitate immediate intervention with a therapy, but may prompt a treating physician to recommend increased monitoring for the development of AMD or related symptoms indicative of the development of AMD. In some embodiments, the method comprises providing a recommendation to reassess the individual for AMD after a period of time. In some embodiments, if the level or the risk score is below the threshold value, the method comprises providing a recommendation to reassess the individual for AMD after a period of time. The period of time may be any suitable period of time. In some embodiments, the period of time is about 1 month to about 24 months. In some embodiments, the period of time is about 1 month to about 3 months, about 1 month to about 6 months, about 1 month to about 9 months, about 1 month to about 12 months, about 1 month to about 18 months, about 1 month to about 24 months, about 3 months to about 6 months, about 3 months to about 9 months, about 3 months to about 12 months, about 3 months to about 18 months, about 3 months to about 24 months, about 6 months to about 9 months, about 6 months to about 12 months, about 6 months to about 18 months, about 6 months to about 24 months, about 9 months to about 12 months, about 9 months to about 18 months, about 9 months to about 24 months, about 12 months to about 18 months, about 12 months to about 24 months, or about 18 months to about 24 months. In some embodiments, the period of time is about 1 month, about 3 months, about 6 months, about 9 months, about 12 months, about 18 months, or about 24 months. In some embodiments, the period of time is at least about 1 month, about 3 months, about 6 months, about 9 months, about 12 months, or about 18 months. In some embodiments, the period of time is at most about 3 months, about 6 months, about 9 months, about 12 months, about 18 months, or about 24 months. The period of time may also be a longer time, such as at least about 2 years, at least about 3 years, at least about 5 years, at least about 10 years, or longer. In some embodiments, it may be recommended that the individual is monitored periodically for development of AMD.

The recommendation to reassess comprises performing an additional test after the period of time, including without limitation assessing the level of RBP4 in the individual or performing any of the other tests or assessments for AMD provided herein, such as a follow up with a physician for a physical examination of the eye.

Additionally, the methods provided herein may further comprise any of the additional tests or diagnostics for assessing the presence of AMD in an individual provided herein, including measurement of the level of RBP4 and comparison to a threshold value as provided herein, measurement of the level of vitamin A and comparison to a threshold value as provided herein, or any other measurement which can be used to diagnose AMD. In some embodiments, the method further comprises performing at least one of color fundus photography, fundus autofluorescence, spectral-domain optical coherence tomography, or microperimetry.

In some embodiments, the method further comprises classifying a progression of age-related macular degeneration. The classification can be based on the level of RBP4, measured as provided herein, or in conjunction with other methods provided herein, such as by classification using Age-Related Eye Disease Study (AREDS) classifications.

### Combination of RBP4 Level/Vitamin A Level and Genomic Variants Predictive of AMD

The biomarker levels (e.g. blood concentration of RBP4 and/or vitamin A) and genomic variants (e.g. ABCA4 variants) provided herein may also be probed simultaneously in order to provide a diagnosis, risk score, or recommendation for treatment of AMD in an individual. In some embodiments, this combinatorial approach yields a diagnosis or risk score that is more reliable than either method alone. In some embodiments, this allows for a certain diagnosis or risk assessment of AMD without a more invasive physical examination of the eye, as the diagnosis or risk score can be calculated from a single sample from the individual (e.g. a blood sample).

In one aspect herein is a method for assessing the likelihood of age-related macular degeneration in an individual in need thereof comprising: obtaining a blood sample from the individual; extracting a protein fraction from the blood sample; extracting a nucleic acid fraction from the blood sample; determining by a first assay a level of retinol binding protein 4 (RBP4) and/or a level of vitamin A from the protein fraction; determining by a second assay an allele frequency of one or more genomic variants from nucleic acids in the nucleic acid fraction; and assessing the likelihood of age-related macular degeneration based on the level of RBP4 and/or the level of vitamin A and the allele frequency of the one or more genomic variants. In some embodiments, the one or more genomic variants comprises at least one of rs4147863, rs2275029, rs1800739, rs4147857, rs4147856, rs1801555, or rs1801574.

In another aspect herein is a method for treating age-related macular degeneration in an individual in need thereof comprising: a) providing a level of retinol binding protein 4 (RBP4) and/or a level of vitamin A, wherein the level was determined by an assay of a protein fraction of a sample from the individual; b) providing an allele frequency of one or more genomic variants, wherein the allele frequency was determined by an assay performed on a nucleic acid fraction of the sample from the individual; and c) administering a therapy based on an assessment of the level of RBP4 and/or a level of vitamin A and the allele frequency of the one or more genomic variants. In some embodiments, the one or more genomic variants comprises at least one of rs4147863, rs2275029, rs1800739, rs4147857, rs4147856, rs1801555, or rs1801574.

In some embodiments, the level of RBP4 is compared to a threshold value of RBP4 as provided elsewhere herein. Any of the threshold values of RBP4 provided herein is used in diagnostic/assessment protocols in conjunction with the detection of genomic variants. Additionally, any of the methods or assays used to determine the level of RBP4 in the sample provided herein are equally applicable to methods which base the diagnosis of AMD, the assessment of risk of developing AMD, or the decision to treat AMD on combinations of genomic variants and RBP4 level.

In some embodiments, the methods herein provide for detecting the presence or absence of the one or more genomic variants. Assessment of any combination or number of genomic variants provided herein can be used in conjunction with the information provided by the level of RBP4 in making the diagnosis, risk assessment, or treatment of AMD provided herein.

In embodiments where both RBP4 levels and genomic variants are measured, the measuring of RBP4 levels by an assay and the determining by a second assay the presence or absence of one or more genomic variants (or the allele frequency thereof) are performed on samples derived from the individual. In some embodiments, the assay and the second assay are performed on the same sample. In some embodiments, the assay and the second assay are performed on different samples derived from the individual. The samples need not be taken from the individual at the same time, nor do the samples need to be of the same type. In some embodiments, the assay and the second assay are performed on the same sample derived from the individual. In some embodiments, the assay and the second assay are performed on two separate samples from the individual. In some embodiments, the two separate samples are the same type of sample (e.g. blood samples). In some embodiments, the two separate samples are different types of samples (e.g. a blood sample and a urine sample). In some embodiments, the two separate samples are two blood samples. The two separate samples need not be taken from the individual at the same time. In some embodiments, the two separate samples are taken from the individual at the same time. In some embodiments, the two separate samples are taken at different times. In some embodiments, the two separate samples are taken at most about 1 day, at most about 2 days, at most about 3 days, at most about 1 week, at most about 2 weeks, at most about 1 month, at most about 2 months, at most about 3 months, or at most about 6 months apart.

Just as methods provided herein based on a measurement RBP4 level or the presence or absence of genomic variants may further comprise additional tests or diagnostics to ascertain the presence of AMD in an individual, so too may methods which utilize both RBP4 level and the presence or absence of genomic variants. In some embodiments, the diagnosis, risk assessment, or decision to administer a therapy is further based on additional criteria, such as the age and medical history of the subject. In some embodiments, the determination is based on the age and/or medical history of the individual. In some embodiments, the determination is based at least partially on the age of the individual. In some embodiments, the determination is based at least partially on the medical history of the individual. In some embodiments, the medical history of the individual can include information such as previous diagnosis with an eye condition, body-mass index (BMI), height, weight, systemic condition including obesity, hyperlipidemia, hyperglycemia, hypertension, diabetes mellitus, cardiovascular disease, or non-alcoholic fatty liver diseases.

Additionally, the methods provided herein may further comprise any of the additional tests or diagnostics for assessing the presence of AMD in an individual provided herein. In some embodiments, the method further comprises performing at least one of color fundus photography, fundus autofluorescence, spectral-domain optical coherence tomography, or microperimetry.

In some embodiments, the method further comprises classifying a progression of age-related macular degeneration. The classification can be based on the level of RBP4, measured as provided herein, or in conjunction with other methods provided herein, such as by classification using Age-Related Eye Disease Study (AREDS) classifications.

In some embodiments, the risk score reflects the probability that the individual will develop AMD at a point in the future based off the measurements provided herein.

In some embodiments, identification of the one or more genomic variants provided herein and measurement of the level or RBP4 in an individual does not necessitate immediate intervention with a therapy, but may prompt a treating physician to recommend increased monitoring for the development of AMD or related symptoms indicative of the development of AMD. In some embodiments, the method comprises providing a recommendation to reassess the individual for AMD after a period of time. In some embodiments, if the level or the risk score is below the threshold value, the method comprises providing a recommendation to reassess the individual for AMD after a period of time. The period of time may be any suitable period of time. In some embodiments, the period of time is about 1 month to about 24 months. In some embodiments, the period of time is about 1 month to about 3 months, about 1 month to about 6 months, about 1 month to about 9 months, about 1 month to about 12 months, about 1 month to about 18 months, about 1 month to about 24 months, about 3 months to about 6 months, about 3 months to about 9 months, about 3 months to about 12 months, about 3 months to about 18 months, about 3 months to about 24 months, about 6 months to about 9 months, about 6 months to about 12 months, about 6 months to about 18 months, about 6 months to about 24 months, about 9 months to about 12 months, about 9 months to about 18 months, about 9 months to about 24 months, about 12 months to about 18 months, about 12 months to about 24 months, or about 18 months to about 24 months. In some embodiments, the period of time is about 1 month, about 3 months, about 6 months, about 9 months, about 12 months, about 18 months, or about 24 months. In some embodiments, the period of time is at least about 1 month, about 3 months, about 6 months, about 9 months, about 12 months, or about 18 months. In some embodiments, the period of time is at most about 3 months, about 6 months, about 9 months, about 12 months, about 18 months, or about 24 months. The period of time may also be a longer time, such as at least about 2 years, at least about 3 years, at least about 5 years, at least about 10 years, or longer. In some embodiments, it may be recommended that the individual is monitored periodically for development of AMD.

The recommendation to reassess comprises the performing an additional test after the period of time, including without limitation assessing the level of RBP4 in the individual or performing any of the other tests or assessments for AMD provided herein, such as a follow up with a physician for a physical examination of the eye.

In some embodiments, a therapy is administered if the genomic variants provided herein are identified in the individual. In some embodiments, the therapy is administered to reduce the level of RBP4 in the individual. In some embodiments, the therapy comprises administering a pharmaceutical composition to the individual. In some embodiments, the therapy comprises administering a pharmaceutical compound to the individual. In some embodiments, the pharmaceutical composition comprises an RBP4 inhibitor. The RBP4 inhibitor may be any of the RBP4 inhibitors provided herein.

### Additional Considerations for Prediction or Diagnosis of AMD

In some embodiments, the determination of the diagnosis of AMD, the risk assessment of developing AMD, or the decision to treat is made based on additional tests which can optionally be performed in addition to measuring the level of RBP4 in the subject and/or determining the presence or absence of the genomic variants provided herein. Thus, the methods provided herein may further provide additional tests used in conjunction with the biomarkers indicative of AMD provided herein.

The methods provided herein may further comprises additional tests used to assess the presence or risk of developing AMD in an individual. A variety of such additional tests and diagnoses are known in the art, including color fundus photograph, fundus autofluorescence, spectral-domain optical coherence tomography, and microperimetry.

In some embodiments, the methods provided herein further comprise analysis of at least one of color fundus photography, fundus autofluorescence, spectral-domain optical coherence tomography, or microperimetry. In some embodiments, the method further comprises analysis of color fundus photography. In some embodiments, the method further comprises analysis of fundus autofluorescence. In some embodiments, the method further comprises spectral-domain optical coherence tomography. In some embodiments, the method further comprises microperimetry. In some embodiments, the diagnosis of AMD, the risk assessment of developing AMD, or the decision to administer to a therapy for AMD is based at least partially on one of these assessments.

Additionally, the methods provided herein may also comprise determining the age or medical history of the individual. In some embodiments, the determination is based at least partially on the age of the individual. In some embodiments, the determination is based at least partially on the medical history of the individual. In some embodiments, the medical history of the individual can include information such as previous diagnosis with an eye condition, body-mass index (BMI), height, weight, systemic condition including obesity, hyperlipidemia, hyperglycemia, hypertension, diabetes mellitus, cardiovascular disease, or non-alcoholic fatty liver diseases.

In some embodiments, the methods herein comprise classifying progression of the AMD. In some embodiments, the classification is based on the Age-Related Eye Disease Study Research categories [Age-Related Eye Disease Study Research G. The Age-Related Eye Disease Study (AREDS): design implications. AREDS report no. 1. Control Clin Trials. 1999; 20(6):573-600) (AREDS)]. AREDS categories include category 1 (defined as a few (1-15), small (<63 μm), or no drusen and without pigment changes); category 2 (early AMD characterized by several small, few intermediate-sized (63-124 μm) drusen, and/or pigmentary changes in one or both eyes); category 3 (intermediate AMD characterized by extensive (20 soft or 65 hard without any soft) intermediate-size drusen, one large (>125 μm) drusen, and/or geographic atrophy not involving the macula in one or both eyes); and category 4 (advanced unilateral AMD consisting of the advanced dry form with geography atrophy involving macula or the exudative form with choroidal neovascularization in one eye).

RBP4 Inhibitory Compounds and Compounds which Lower RBP4 in the Blood

Provided herein in some embodiments are RBP4 inhibitory compounds, compounds which lower RBP4 levels in the blood, and pharmaceutical compositions comprising said compounds. In some embodiments, the compounds are RBP4 inhibitors. In some embodiments, the compounds lower RBP4 levels in the blood. The subject compounds and compositions are useful for inhibiting RPB4 and for the treatment of various disorders, including age-related macular degeneration and STGD. In some embodiments, an individual is administered a therapy comprising an RBP4 inhibitory compound, such as those provided herein, upon diagnosis with AMD or STGD or an assessment that the individual is at risk of developing AMD or STGD with the methods provided herein. Examples of RBP4 inhibitor compounds can be found in US Publication No. US 2010/0292206; PCT Publication No. WO2010119992; US Publication No. US2011/0251187, US Publication No. US2018/0237404; U.S. Pat. Nos. 10,273,243; 8,980,924; 9,637,450; 9,944,644; 9,938,291; 10,072,016; and PCT Publication No. WO2018232154, each of which is incorporated by reference.

Some embodiments provided herein describe an RBP4 inhibitor, or a pharmaceutically acceptable salt, solvate, polymorph, prodrug, metabolite, N-oxide, stereoisomer, or isomer thereof, for use in treating age-related macular degeneration, having the structure of Formula (I):

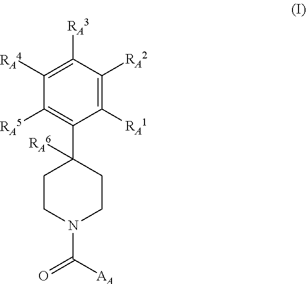

wherein:
   each R_{A}¹, R_{A}², R_{A}³, R_{A}⁴, and R_{A}⁵ is independently halogen, optionally substituted alkyl, optionally substituted cycloalkyl, optionally substituted heterocyclyl, optionally substituted heterocyclolalkyl, —COR_{A}⁷, —CON(R_{A}⁷)₂, optionally substituted (C₀-C₄ alkylene)-CN, optionally substituted (C₀-C₄ alkylene)-OR_{A}⁷, optionally substituted (C₀-C₄ alkylene)-N(R_{A}⁷)₂, optionally substituted (C₀-C₄ alkylene)N(R_{A}⁸)—COR_{A}⁷, optionally substituted (C₀-C₄ alkylene)-SO₂N(R_{A}⁷)₂, optionally substituted (C₀-C₄ alkylene)-SO₂R_{A}⁷, optionally substituted (C₀-C₄ alkylene)N(R_{A}⁸)—SO₂N(R_{A}⁷)₂, or optionally substituted (C₀-C₄ alkylene)N(R_{A}⁸)—SO₂R_{A}⁷;
      each R_{A}⁷ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two
         R_{A}¹¹ groups together with the nitrogen to which they are attached join to form an optionally substituted N-heterocyclyl;
      each R_{A}⁸ is independently selected from H or optionally substituted alkyl;
   R_{A}⁶ is —H, —OH, optionally substituted alkyl, or halogen;
   p is 0, 1, 2, 3, 4, or 5;
   A_{A} has the structure:

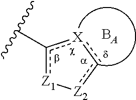

wherein:
   α, β, χ, and δ are each independently absent or present, and when present each is a bond;
   X is C;
   Z₁ is S, O, or N;
   Z₂ is S, O, N, or NR_{A}⁹;
   R_{A}⁹ is H, optionally substituted alkyl, or oxetane; and
   B_{A} is a substituted or unsubstituted fused 5-, 6-, or 7-membered ring structure; or
   a pharmaceutically acceptable salt thereof.

In certain embodiments, the compound of Formula (I) has the structure

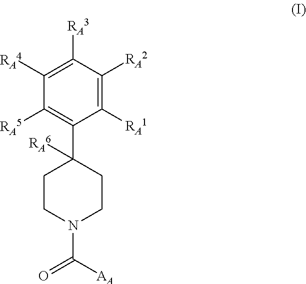

wherein
   R_{A}¹, R_{A}², R_{A}³, R_{A}⁴, and R_{A}⁵ are each independently H, halogen, CF₃ or C₁-C₄ alkyl,
   wherein two or more of R_{A}¹, R_{A}², R_{A}³, R_{A}⁴, and R_{A}⁵ are other than H;
   R_{A}⁶ is H, OH, or halogen; and
   A_{A} has the structure:

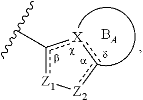

wherein
   α, β, χ, and δ are each independently absent or present, and when present each is a bond;
   X is C or N;
   Z₁ is N;
   Z₂ is N or NR_{A}⁹,
   wherein R_{A}⁹ is H, C₁-C₄ alkyl, or oxetane;
B_{A} is a substituted or unsubstituted 5, 6, or 7 membered ring structure; or
a pharmaceutically acceptable salt thereof.

In some embodiments, the compound of Formula (I) is 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)ethan-1-one. In certain embodiments, the compound of Formula (I) is 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)ethan-1-one; 1-(3-(4-(3,4-difluoro-2-(trifluoromethyl)phenyl)piperidine-1-carbonyl)-4,6-dihydropyrrolo[3,4-c]pyrazol-5(1H)-yl)ethan-1-one; (4-(3-fluoro-2,5-bis(trifluoromethyl)phenyl)piperidin-1-yl)(4,5,6,7-tetrahydro-1H-pyrazolo[4,3-c]pyridin-3-yl)methanone; (4-(2-chloro-3-fluorophenyl)piperidin-1-yl)(5-(2-methoxyethyl)-4,5,6,7-tetrahydro-1H-pyrazolo[4,3-c]pyridin-3-yl)methanone; (4-(2-chloro-3-fluorophenyl)piperidin-1-yl)(5-(3,3,3-trifluoropropyl)-4,5,6,7-tetrahydro-1H-pyrazolo[4,3-c]pyridin-3-yl)methanone; (4-(2-chloro-3-fluorophenyl)piperidin-1-yl)(5-(2,2,2-trifluoroethyl)-4,5, 6,7-tetrahydro-1H-pyrazolo[4,3-c]pyridin-3-yl)methanone; (4-(2-chloro-3-fluorophenyl)piperidin-1-yl)(5-(oxetan-3-yl)-4,5,6,7-tetrahydro-1H-pyrazolo[4,3-c]pyridin-3-yl)methanone; (4-(4-fluoro-2-(trifluoromethyl)phenyl)piperidin-1-yl)(4,5,6,7-tetrahydro-1H-pyrazolo[4,3-c]pyridin-3-yl)methanone; (4-(4-fluoro-2-(trifluoromethyl)phenyl)piperidin-1-yl)(4,5,6,7-tetrahydro-1H-pyrazolo[3,4-c]pyridin-3-yl)methanone; (4-(2-chloro-3-fluorophenyl)piperidin-1-yl)(5-(cyclopropylmethyl)-4,5,6,7-tetrahydro-1H-pyrazolo[4,3-c]pyridin-3-yl)methanone; (4-(2-chloro-3-fluorophenyl)piperidin-1-yl)(5-ethyl-4,5,6,7-tetrahydro-1H-pyrazolo[4,3-c]pyridin-3-yl)methanone; (4-(3,4-difluoro-2-(trifluoromethyl)phenyl)piperidin-1-yl)(6-ethyl-4,5, 6,7-tetrahydro-1H-pyrazolo[3,4-c]pyridin-3-yl)methanone; (4-(4-fluoro-2-(trifluoromethyl)phenyl)piperidin-1-yl)(5-(methylsulfonyl)-4,5,6,7-tetrahydro-1H-pyrazolo[4,3-c]pyridin-3-yl)methanone; 1-(3-(4-(2-fluoro-6-(trifluoromethyl)phenyl)piperidine-1-carbonyl)-1,4,5,7-tetrahydro-6H-pyrazolo[3,4-c]pyridin-6-yl)ethan-1-one; (4-(3-fluoro-2,5-bis(trifluoromethyl)phenyl)piperidin-1-yl)(4,5,6,7-tetrahydro-1H-pyrazolo[3,4-c]pyridin-3-yl)methanone; (4-(2-chloro-3-fluorophenyl)piperidin-1-yl)(6-(cyclopropylmethyl)-4,5,6,7-tetrahydro-1H-pyrazolo[3,4-c]pyridin-3-yl)methanone; (4-(3,5-bis(trifluoromethyl)phenyl)piperidin-1-yl)(5-(methylsulfonyl)-4,5,6,7-tetrahydro-1H-pyrazolo[4,3-c]pyridin-3-yl)methanone; (4-(2-chloro-3-fluorophenyl)piperidin-1-yl)(5-(methylsulfonyl)-4,5,6,7-tetrahydro-1H-pyrazolo[4,3-c]pyridin-3-yl)methanone; (4-(2-chloro-3-fluorophenyl)piperidin-1-yl)(6-(oxetan-3-yl)-4,5,6,7-tetrahydro-1H-pyrazolo[3,4-c]pyridin-3-yl)methanone; 3-(4-(2-chloro-3-fluorophenyl)piperidine-1-carbonyl)-1,4,5,7-tetrahydro-6H-pyrazolo[3,4-c]pyridine-6-carbonitrile(4-(5-fluoro-2-(trifluoromethyl)phenyl)piperidin-1-yl)(5-(methylsulfonyl)-4,5,6,7-tetrahydro-1H-pyrazolo[4,3-c]pyridin-3-yl)methanone; 3-(4-(3,5-difluoro-2-(trifluoromethyl)phenyl)piperidine-1-carbonyl)-N-methyl-1,4,5,7-tetrahydro-6H-pyrazolo[3,4-c]pyridine-6-carboxamide(6-(cyclopropylmethyl)-4,5,6,7-tetrahydro-1H-pyrazolo[3,4-c]pyridin-3-yl)(4-(3,4-difluoro-2-(trifluoromethyl)phenyl)piperidin-1-yl)methanone; methyl 3-(4-(3,5-difluoro-2-(trifluoromethyl)phenyl)piperidine-1-carbonyl)-1,4,5,7-tetrahydro-6H-pyrazolo[3,4-c]pyridine-6-carboxylate; (4-(3, 5-difluoro-2-(trifluoromethyl)phenyl)piperidin-1-yl)(5-(3,3,3-trifluoropropyl)-4,5,6,7-tetrahydro-1H-pyrazolo[4,3-c]pyridin-3-yl)methanone; (4-(3,4-difluoro-2-(trifluoromethyl)phenyl)piperidin-1-yl)(5-neopentyl-4,5,6,7-tetrahydro-1H-pyrazolo[4,3-c]pyridin-3-yl)methanone; (4-(2-chloro-5-fluorophenyl)piperidin-1-yl)(5-(methylsulfonyl)-4,5,6,7-tetrahydro-1H-pyrazolo[4,3-c]pyridin-3-yl)methanone; (4-(3,5-difluoro-2-(trifluoromethyl)phenyl)piperidin-1-yl)(6-(2,2,2-trifluoroethyl)-4,5,6,7-tetrahydro-1H-pyrazolo[3,4-c]pyridin-3-yl)methanone; 3-(4-(3,5-difluoro-2-(trifluoromethyl)phenyl)piperidine-1-carbonyl)-1,4,5,7-tetrahydro-6H-pyrazolo[3,4-c]pyridine-6-carbonitrile; (4-(3,5-difluoro-2-(trifluoromethyl)phenyl)piperidin-1-yl)(6-(oxetan-3-yl)-4,5,6,7-tetrahydro-1H-pyrazolo[3,4-c]pyridin-3-yl)methanone; 3-(4-(3,4-difluoro-2-(trifluoromethyl)phenyl)piperidine-1-carbonyl)-N-methyl-1,4,5,7-tetrahydro-6H-pyrazolo[3,4-c]pyridine-6-carboxamide; (4-(3,4-difluoro-2-(trifluoromethyl)phenyl)piperidin-1-yl)(6-neopentyl-4,5,6,7-tetrahydro-1H-pyrazolo[3,4-c]pyridin-3-yl)methanone; (4-(3,4-difluoro-2-(trifluoromethyl)phenyl)piperidin-1-yl)(6-(2,2,2-trifluoroethyl)-4,5, 6,7-tetrahydro-1H-pyrazolo[3,4-c]pyridin-3-yl)methanone; methyl 3-(4-(2-chloro-3-fluorophenyl)piperidine-1-carbonyl)-1,4,5,7-tetrahydro-6H-pyrazolo[3,4-c]pyridine-6-carboxylate; 1-(3-(4-(3,5-difluoro-2-(trifluoromethyl)phenyl)piperidine-1-carbonyl)-1,4,5,7-tetrahydro-6H-pyrazolo[3,4-c]pyridin-6-yl)ethan-1-one; (6-(cyclopropylmethyl)-4,5,6,7-tetrahydro-1H-pyrazolo[3,4-c]pyridin-3-yl)(4-(3,5-difluoro-2-(trifluoromethyl)phenyl)piperidin-1-yl)methanone; (4-(3,4-difluoro-2-(trifluoromethyl)phenyl)piperidin-1-yl)(6-(2-methoxyethyl)-4,5, 6,7-tetrahydro-1H-pyrazolo[3,4-c]pyridin-3-yl)methanone; 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)-3-methylbutan-1-one; 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)propan-1-one; 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)-2-methylpropan-1-one; 3-(4-(3,4-difluoro-2-(trifluoromethyl)phenyl)piperidine-1-carbonyl)-1,4,5,7-tetrahydro-6H-pyrazolo[3,4-c]pyridine-6-carbonitrile; (4-(3,4-difluoro-2-(trifluoromethyl)phenyl)piperidin-1-yl)(6-(3,3,3-trifluoropropyl)-4,5,6,7-tetrahydro-1H-pyrazolo[3,4-c]pyridin-3-yl)methanone; (4-(3,4-difluoro-2-(trifluoromethyl)phenyl)piperidin-1-yl)(5-(2-methoxyethyl)-4,5,6,7-tetrahydro-1H-pyrazolo[4,3-c]pyridin-3-yl)methanone; (4-(3,4-difluoro-2-(trifluoromethyl)phenyl)piperidin-1-yl)(5-(3,3,3-trifluoropropyl)-4,5,6,7-tetrahydro-1H-pyrazolo[4,3-c]pyridin-3-yl)methanone; 3-(4-(3,4-difluoro-2-(trifluoromethyl)phenyl)piperidine-1-carbonyl)-N-methyl-1,4,6,7-tetrahydro-5H-pyrazolo[4,3-c]pyridine-5-carboxamide; (4-(3,4-difluoro-2-(trifluoromethyl)phenyl)piperidin-1-yl)(6-(oxetan-3-yl)-4,5,6,7-tetrahydro-1H-pyrazolo[3,4-c]pyridin-3-yl)methanone; methyl 3-(4-(3,4-difluoro-2-(trifluoromethyl)phenyl)piperidine-1-carbonyl)-1,4,5,7-tetrahydro-6H-pyrazolo[3,4-c]pyridine-6-carboxylate; 2-(3-(4-(3,4-difluoro-2-(trifluoromethyl)phenyl)piperidine-1-carbonyl)-1,4,5,7-tetrahydro-6H-pyrazolo[3,4-c]pyridin-6-yl)acetic acid; (4-(3,4-difluoro-2-(trifluoromethyl)phenyl)piperidin-1-yl)(5-(oxetan-3-yl)-4,5,6,7-tetrahydro-1H-pyrazolo[4,3-c]pyridin-3-yl)methanone; (5-(cyclopropylmethyl)-4,5,6,7-tetrahydro-1H-pyrazolo[4,3-c]pyridin-3-yl)(4-(3,4-difluoro-2-(trifluoromethyl)phenyl)piperidin-1-yl)methanone; (4-(3,4-difluoro-2-(trifluoromethyl)phenyl)piperidin-1-yl)(5-ethyl-4,5,6,7-tetrahydro-1H-pyrazolo[4,3-c]pyridin-3-yl)methanone; 3-(4-(3-fluoro-2-(trifluoromethyl)phenyl)piperidine-1-carbonyl)-N-methyl-1,4,6,7-tetrahydro-5H-pyrazolo[4,3-c]pyridine-5-carboxamide; (4-(3,4-difluoro-2-(trifluoromethyl)phenyl)piperidin-1-yl)(6-methyl-4,5,6,7-tetrahydro-1H-pyrazolo[3,4-c]pyridin-3-yl)methanone; methyl 3-(4-(3,5-difluoro-2-(trifluoromethyl)phenyl)piperidine-1-carbonyl)-1,4,6,7-tetrahydro-5H-pyrazolo[4,3-c]pyridine-5-carboxylate; (4-(3,5-difluoro-2-(trifluoromethyl)phenyl)piperidin-1-yl)(5-ethyl-4,5,6,7-tetrahydro-1H-pyrazolo[4,3-c]pyridin-3-yl)methanone; (4-(3,5-difluoro-2-(trifluoromethyl)phenyl)piperidin-1-yl)(5-(2-methoxyethyl)-4,5,6,7-tetrahydro-1H-pyrazolo[4,3-c]pyridin-3-yl)methanone; 3-(4-(3,4-difluoro-2-(trifluoromethyl)phenyl)piperidine-1-carbonyl)-1,4,6,7-tetrahydro-5H-pyrazolo[4,3-c]pyridine-5-carbonitrile; methyl 3-(4-(3,4-difluoro-2-(trifluoromethyl)phenyl)piperidine-1-carbonyl)-1,4,6,7-tetrahydro-5H-pyrazolo[4,3-c]pyridine-5-carboxylate; (4-(3-fluoro-2-(trifluoromethyl)phenyl)piperidin-1-yl)(6-(3,3,3-trifluoropropyl)-4,5,6,7-tetrahydro-1H-pyrazolo[3,4-c]pyridin-3-yl)methanone; (6-ethyl-4,5,6,7-tetrahydro-1H-pyrazolo[3,4-c]pyridin-3-yl)(4-(3-fluoro-2-(trifluoromethyl)phenyl)piperidin-1-yl)methanone; (4-(3-fluoro-2-(trifluoromethyl)phenyl)piperidin-1-yl)(5-(oxetan-3-yl)-4,5,6,7-tetrahydro-1H-pyrazolo[4,3-c]pyridin-3-yl)methanone; (4-(3-fluoro-2-(trifluoromethyl)phenyl)piperidin-1-yl)(5-(2-methoxyethyl)-4,5,6,7-tetrahydro-1H-pyrazolo[4,3-c]pyridin-3-yl)methanone; (4-(3-fluoro-2-(trifluoromethyl)phenyl)piperidin-1-yl)(5-(3,3,3-trifluoropropyl)-4,5,6,7-tetrahydro-1H-pyrazolo[4,3-c]pyridin-3-yl)methanone; (4-(2-chloro-5-fluorophenyl)piperidin-1-yl)(4,5,6,7-tetrahydro-1H-pyrazolo[3,4-c]pyridin-3-yl)methanone; imidazo[1,2-a]pyridin-2-yl(4-(2-(trifluoromethyl)phenyl)piperidin-1-yl)methanone; (4-(5-fluoro-2-(trifluoromethyl)phenyl)piperidin-1-yl)(4,5,6,7-tetrahydro-1H-pyrazolo[4,3-c]pyridin-3-yl)methanone; 1-(3-(4-(3,4-difluoro-2-(trifluoromethyl)phenyl)piperidine-1-carbonyl)-1,4,6,7-tetrahydro-5H-pyrazolo[4,3-c]pyridin-5-yl)ethan-1-one; (5-ethyl-4,5,6,7-tetrahydro-1H-pyrazolo[4,3-c]pyridin-3-yl)(4-(3-fluoro-2-(trifluoromethyl)phenyl)piperidin-1-yl)methanone; 3-(4-(3-fluoro-2-(trifluoromethyl)phenyl)piperidine-1-carbonyl)-N-methyl-1,4,5,7-tetrahydro-6H-pyrazolo[3,4-c]pyridine-6-carboxamide; (4-(3-fluoro-2-(trifluoromethyl)phenyl)piperidin-1-yl)(5-(methylsulfonyl)-4,5,6,7-tetrahydro-1H-pyrazolo[4,3-c]pyridin-3-yl)methanone; (4-(3,5-difluoro-2-(trifluoromethyl)phenyl)piperidin-1-yl)(4,5,6,7-tetrahydro-1H-pyrazolo[4,3-c]pyridin-3-yl)methanone; (4-(3,5-difluoro-2-(trifluoromethyl)phenyl)piperidin-1-yl)(4,5,6,7-tetrahydro-1H-pyrazolo[3,4-c]pyridin-3-yl)methanone; (4-(2-chloro-3-fluorophenyl)piperidin-1-yl)(4,5,6,7-tetrahydro-1H-pyrazolo[4,3-c]pyridin-3-yl)methanone; (4-(2-chloro-3-fluorophenyl)piperidin-1-yl)(4,5,6,7-tetrahydro-1H-pyrazolo[3,4-c]pyridin-3-yl)methanone; (4-(3,4-difluoro-2-(trifluoromethyl)phenyl)piperidin-1-yl)(4,5,6,7-tetrahydro-1H-pyrazolo[4,3-c]pyridin-3-yl)methanone; (4-(3,4-difluoro-2-(trifluoromethyl)phenyl)piperidin-1-yl)(4,5,6,7-tetrahydro-1H-pyrazolo[3,4-c]pyridin-3-yl)methanone; (4-(3,5-bis(trifluoromethyl)phenyl)piperidin-1-yl)(4,5,6,7-tetrahydro-1H-pyrazolo[4,3-c]pyridin-3-yl)methanone; (4-(3,5-bis(trifluoromethyl)phenyl)piperidin-1-yl)(4,5,6,7-tetrahydro-1H-pyrazolo[3,4-c]pyridin-3-yl)methanone; (4-(3-fluoro-2-(trifluoromethyl)phenyl)piperidin-1-yl)(4,5,6,7-tetrahydro-1H-pyrazolo[4,3-c]pyridin-3-yl)methanone; (4-(3-fluoro-2-(trifluoromethyl)phenyl)piperidin-1-yl)(4,5,6,7-tetrahydro-1H-pyrazolo[3,4-c]pyridin-3-yl)methanone; 1-(3-(4-(2-chloro-3-fluorophenyl)piperidine-1-carbonyl)-1,4,5,7-tetrahydro-6H-pyrazolo[3,4-c]pyridin-6-yl)ethan-1-one; 1-(3-(4-(3-fluoro-2-(trifluoromethyl)phenyl)piperidine-1-carbonyl)-1,4,5,7-tetrahydro-6H-pyrazolo[3,4-c]pyridin-6-yl)ethan-1-one; 1-(3-(4-(5-fluoro-2-(trifluoromethyl)phenyl)piperidine-1-carbonyl)-1,4,5,7-tetrahydro-6H-pyrazolo[3,4-c]pyridin-6-yl)ethan-1-one; 1-(3-(4-(3,5-bis(trifluoromethyl)phenyl)piperidine-1-carbonyl)-1,4,5,7-tetrahydro-6H-pyrazolo[3,4-c]pyridin-6-yl)ethan-1-one; 1-(3-(4-(4-fluoro-2-(trifluoromethyl)phenyl)piperidine-1-carbonyl)-1,4,5,7-tetrahydro-6H-pyrazolo[3,4-c]pyridin-6-yl)ethan-1-one; (4-(2-fluoro-6-(trifluoromethyl)phenyl)piperidin-1-yl)(4,5,6,7-tetrahydro-1H-pyrazolo[4,3-c]pyridin-3-yl)methanone; (4-(3-fluoro-2-(trifluoromethyl)phenyl)piperidin-1-yl)(5-(2,2,2-trifluoroethyl)-4,5,6,7-tetrahydro-1H-pyrazolo[4,3-c]pyridin-3-yl)methanone; (5-(cyclopropylmethyl)-4,5,6,7-tetrahydro-1H-pyrazolo[4,3-c]pyridin-3-yl)(4-(3-fluoro-2-(trifluoromethyl)phenyl)piperidin-1-yl)methanone; methyl 3-(4-(3-fluoro-2-(trifluoromethyl)phenyl)piperidine-1-carbonyl)-1,4,6,7-tetrahydro-5H-pyrazolo[4,3-c]pyridine-5-carboxylate; 3-(4-(3-fluoro-2-(trifluoromethyl)phenyl)piperidine-1-carbonyl)-1,4,6,7-tetrahydro-5H-pyrazolo[4,3-c]pyridine-5-carbonitrile; 1-(3-(4-(2-chloro-5-fluorophenyl)piperidine-1-carbonyl)-1,4,5,7-tetrahydro-6H-pyrazolo[3,4-c]pyridin-6-yl)ethan-1-one; (4-(2-fluoro-6-(trifluoromethyl)phenyl)piperidin-1-yl)(4,5,6,7-tetrahydro-1H-pyrazolo[3,4-c]pyridin-3-yl)methanone; tert-butyl 2-(3-(4-(3,4-difluoro-2-(trifluoromethyl)phenyl)piperidine-1-carbonyl)-1,4,5,7-tetrahydro-6H-pyrazolo[3,4-c]pyridin-6-yl)acetate; tert-butyl 3-(4-(3,5-difluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(3,5-difluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(2-fluoro-6-(trifluoromethyl)phenyl)piperidine-1-carbonyl)-1,4,6,7-tetrahydro-5H-pyrazolo[4,3-c]pyridine-5-carboxylate; tert-butyl 3-(4-(5-fluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(5-fluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(2-chloro-3-fluorophenyl)piperidine-1-carbonyl)-1,4,6,7-tetrahydro-5H-pyrazolo[4,3-c]pyridine-5-carboxylate; tert-butyl 3-(4-(2-fluoro-6-(trifluoromethyl)phenyl)piperidine-1-carbonyl)-1,4,5,7-tetrahydro-6H-pyrazolo[3,4-c]pyridine-6-carboxylatetert-butyl 3-(4-(3,5-bis(trifluoromethyl)phenyl)piperidine-1-carbonyl)-1,4,6,7-tetrahydro-5H-pyrazolo[4,3-c]pyridine-5-carboxylate; tert-butyl 3-(4-(3,5-bis(trifluoromethyl)phenyl)piperidine-1-carbonyl)-1,4,5,7-tetrahydro-6H-pyrazolo[3,4-c]pyridine-6-carboxylate; tert-butyl 3-(4-(2-chloro-5-fluorophenyl)piperidine-1-carbonyl)-1,4,6,7-tetrahydro-5H-pyrazolo[4,3-c]pyridine-5-carboxylate; tert-butyl 3-(4-(2-chloro-5-fluorophenyl)piperidine-1-carbonyl)-1,4,5,7-tetrahydro-6H-pyrazolo[3,4-c]pyridine-6-carboxylate; tert-butyl 3-(4-(4-fluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(3-fluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(3,4-difluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(3,4-difluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(3-fluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(4-fluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(2-chloro-3-fluorophenyl)piperidine-1-carbonyl)-1,4,5,7-tetrahydro-6H-pyrazolo[3,4-c]pyridine-6-carboxylate; (6,6-dimethyl-1,4,6,7-tetrahydropyrano[4,3-c]pyrazol-3-yl)(4-(2-(trifluoromethyl)phenyl)piperidin-1-yl)methanone; (6,6-dioxido-1,4,5,7-tetrahydrothiopyrano[3,4-c]pyrazol-3-yl)(4-(2-(trifluoromethyl)phenyl)piperidin-1-yl)methanone; (1,4,6,7-tetrahydropyrano[4,3-c]pyrazol-3-yl)(4-(2-(trifluoromethyl)phenyl)piperidin-1-yl)methanone; (1,4,5,7-tetrahydropyrano[3,4-c]pyrazol-3-yl)(4-(2-(trifluoromethyl)phenyl)piperidin-1-yl)methanone; (1-methyl-4,5,6,7-tetrahydro-1H-pyrazolo[4,3-c]pyridin-3-yl)(4-(2-(trifluoromethyl)phenyl)piperidin-1-yl)methanone; (1-methyl-4,5,6,7-tetrahydro-1H-pyrazolo[3,4-c]pyridin-3-yl)(4-(2-(trifluoromethyl)phenyl)piperidin-1-yl)methanone; 1-ethyl-N,N-dimethyl-3-(4-(2-(trifluoromethyl)phenyl)piperidine-1-carbonyl)-1,4,5,7-tetrahydro-6H-pyrazolo[3,4-c]pyridine-6-carboxamide; (5-(2,2,2-trifluoroethyl)-4,5,6,7-tetrahydro-1H-pyrazolo[4,3-c]pyridin-3-yl)(4-(2-(trifluoromethyl)phenyl)piperidin-1-yl)methanone; (6-(2,2,2-trifluoroethyl)-4,5,6,7-tetrahydro-1H-pyrazolo[3,4-c]pyridin-3-yl)(4-(2-(trifluoromethyl)phenyl)piperidin-1-yl)methanone; (5-chloro-1H-indazol-3-yl)(4-(2-(trifluoromethyl)phenyl)piperidin-1-yl)methanone; (1H-pyrazolo[3,4-b]pyridin-3-yl)(4-(2-(trifluoromethyl)phenyl)piperidin-1-yl)methanone; (6-chloro-1H-indazol-3-yl)(4-(2-(trifluoromethyl)phenyl)piperidin-1-yl)methanone; (5-(methylsulfonyl)-4,5,6,7-tetrahydro-1H-pyrazolo[4,3-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,6,7-tetrahydro-5H-pyrazolo[4,3-c]pyridin-5-yl)ethan-1-one; (4,5,6,7-tetrahydro-1H-pyrazolo[4,3-c]pyridin-3-yl)(4-(2-(trifluoromethyl)phenyl)piperidin-1-yl)methanone; (6-fluoro-1-(oxetan-3-yl)-1H-indazol-3-yl)(4-(2-(trifluoromethyl)phenyl)piperidin-1-yl)methanone; (1-ethyl-6-fluoro-1H-indazol-3-yl)(4-(2-(trifluoromethyl)phenyl)piperidin-1-yl)methanone; (6-fluoro-1-isopropyl-1H-indazol-3-yl)(4-(2-(trifluoromethyl)phenyl)piperidin-1-yl)methanone; 1-(3-(4-fluoro-4-(2-(trifluoromethyl)phenyl)piperidine-1-carbonyl)-1,4,6,7-tetrahydro-5H-pyrazolo[4,3-c]pyridin-5-yl)ethan-1-one; (5-fluoro-1-methyl-1H-indazol-3-yl)(4-(2-(trifluoromethyl)phenyl)piperidin-1-yl)methanone; (6-fluoro-1H-indazol-3-yl)(4-(2-(trifluoromethyl)phenyl)piperidin-1-yl)methanone; (6-methyl-4,5,6,7-tetrahydro-1H-pyrazolo[3,4-c]pyridin-3-yl)(4-(2-(trifluoromethyl)phenyl)piperidin-1-yl)methanone; (6-(methylsulfonyl)-4,5,6,7-tetrahydro-1H-pyrazolo[3,4-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,5,7-tetrahydro-6H-pyrazolo[3,4-c]pyridin-6-yl)ethan-1-one; (5-fluoro-1H-indazol-3-yl)(4-(2-(trifluoromethyl)phenyl)piperidin-1-yl)methanone; (5-((chloromethyl)sulfonyl)-4,5,6,7-tetrahydro-1H-pyrazolo[4,3-c]pyridin-3-yl)(4-(2-(trifluoromethyl)phenyl)piperidin-1-yl)methanone; (5-(2-methoxyethyl)-4,5,6,7-tetrahydro-1H-pyrazolo[4,3-c]pyridin-3-yl)(4-(2-(trifluoromethyl)phenyl)piperidin-1-yl)methanone; (4-fluoro-4-(2-(trifluoromethyl)phenyl)piperidin-1-yl)(4,5,6,7-tetrahydro-1H-pyrazolo[4,3-c]pyridin-3-yl)methanone; 1-(3-(4-(4-fluoro-2-(trifluoromethyl)phenyl)piperidine-1-carbonyl)-1,4,5,7-tetrahydro-6H-pyrazolo[3,4-c]pyridin-6-yl)ethan-1-one; (1-ethyl-5-fluoro-1H-indazol-3-yl)(4-(2-(trifluoromethyl)phenyl)piperidin-1-yl)methanone; (6-fluoro-1-methyl-1H-indazol-3-yl)(4-(2-(trifluoromethyl)phenyl)piperidin-1-yl)methanone; 3-(4-(2-(trifluoromethyl)phenyl)piperidine-1-carbonyl)-1,4,5,7-tetrahydro-6H-pyrazolo[4,3-c]pyridin-6-one; 3-(4-(2-(trifluoromethyl)phenyl)piperidine-1-carbonyl)-1,4,6,7-tetrahydro-5H-pyrazolo[3,4-c]pyridin-5-one; 6-methyl-3-(4-(2-(trifluoromethyl)phenyl)piperidine-1-carbonyl)-1,4,6,7-tetrahydro-5H-pyrazolo[3,4-c]pyridin-5-one; 5-methyl-3-(4-(2-(trifluoromethyl)phenyl)piperidine-1-carbonyl)-1,4,5,7-tetrahydro-6H-pyrazolo[4,3-c]pyridin-6-one; (5,5-dioxido-1,4,6,7-tetrahydrothiopyrano[4,3-c]pyrazol-3-yl)(4-(2-(trifluoromethyl)phenyl)piperidin-1-yl)methanone; (1-methyl-5-(methylsulfonyl)-4,5,6,7-tetrahydro-1H-pyrazolo[4,3-c]pyridin-3-yl)(4-(2-(trifluoromethyl)phenyl)piperidin-1-yl)methanone; (1-methyl-6-(methylsulfonyl)-4,5,6,7-tetrahydro-1H-pyrazolo[3,4-c]pyridin-3-yl)(4-(2-(trifluoromethyl)phenyl)piperidin-1-yl)methanone; 1-(1-ethyl-3-(4-(2-(trifluoromethyl)phenyl)piperidine-1-carbonyl)-1,4,5,7-tetrahydro-6H-pyrazolo[3,4-c]pyridin-6-yl)ethan-1-one; (5-(methoxymethyl)-4,5,6,7-tetrahydro-1H-pyrazolo[4,3-c]pyridin-3-yl)(4-(2-(trifluoromethyl)phenyl)piperidin-1-yl)methanone; (6-(methoxymethyl)-4,5,6,7-tetrahydro-1H-pyrazolo[3,4-c]pyridin-3-yl)(4-(2-(trifluoromethyl)phenyl)piperidin-1-yl)methanone; (5-methoxy-1H-indazol-3-yl)(4-(2-(trifluoromethyl)phenyl)piperidin-1-yl)methanone; (5-(oxetan-3-yl)-4,5,6,7-tetrahydro-1H-pyrazolo[4,3-c]pyridin-3-yl)(4-(2-(trifluoromethyl)phenyl)piperidin-1-yl)methanone; (5-isobutyl-4,5,6,7-tetrahydro-1H-pyrazolo[4,3-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,6,7-tetrahydro-5H-pyrazolo[4,3-c]pyridin-5-yl)propan-1-one; (5-ethyl-4,5,6,7-tetrahydro-1H-pyrazolo[4,3-c]pyridin-3-yl)(4-(2-(trifluoromethyl)phenyl)piperidin-1-yl)methanone; 3-methyl-1-(3-(4-(2-(trifluoromethyl)phenyl)piperidine-1-carbonyl)-1,4,6,7-tetrahydro-5H-pyrazolo[4,3-c]pyridin-5-yl)butan-1-one; 2-methyl-1-(3-(4-(2-(trifluoromethyl)phenyl)piperidine-1-carbonyl)-1,4,6,7-tetrahydro-5H-pyrazolo[4,3-c]pyridin-5-yl)propan-1-one; 2,2-dimethyl-1-(3-(4-(2-(trifluoromethyl)phenyl)piperidine-1-carbonyl)-1,4,6,7-tetrahydro-5H-pyrazolo[4,3-c]pyridin-5-yl)propan-1-one; (5-(isopropylsulfonyl)-4,5,6,7-tetrahydro-1H-pyrazolo[4,3-c]pyridin-3-yl)(4-(2-(trifluoromethyl)phenyl)piperidin-1-yl)methanone; (5-(isobutylsulfonyl)-4,5,6,7-tetrahydro-1H-pyrazolo[4,3-c]pyridin-3-yl)(4-(2-(trifluoromethyl)phenyl)piperidin-1-yl)methanone; (5-(ethylsulfonyl)-4,5,6,7-tetrahydro-1H-pyrazolo[4,3-c]pyridin-3-yl)(4-(2-(trifluoromethyl)phenyl)piperidin-1-yl)methanone; 3-(4-(2-(trifluoromethyl)phenyl)piperidine-1-carbonyl)-1H-indazole-5-carbonitrile; (7-chloro-1H-indazol-3-yl)(4-(2-(trifluoromethyl)phenyl)piperidin-1-yl)methanone; (5,6-difluoro-1H-indazol-3-yl)(4-(2-(trifluoromethyl)phenyl)piperidin-1-yl)methanone; (6-(2-methoxyethyl)-4,5,6,7-tetrahydro-1H-pyrazolo[3,4-c]pyridin-3-yl)(4-(2-(trifluoromethyl)phenyl)piperidin-1-yl)methanone; 3,3,3-trifluoro-1-(3-(4-(2-(trifluoromethyl)phenyl)piperidine-1-carbonyl)-1,4,5,7-tetrahydro-6H-pyrazolo[3,4-c]pyridin-6-yl)propan-1-one; (5-(tert-butyl)-4,5,6,7-tetrahydro-1H-pyrazolo[4,3-c]pyridin-3-yl)(4-(2-(trifluoromethyl)phenyl)piperidin-1-yl)methanone; (5-isopropyl-4,5,6,7-tetrahydro-1H-pyrazolo[4,3-c]pyridin-3-yl)(4-(2-(trifluoromethyl)phenyl)piperidin-1-yl)methanone; N-methyl-3-(4-(2-(trifluoromethyl)phenyl)piperidine-1-carbonyl)-1,4,6,7-tetrahydro-5H-pyrazolo[4,3-c]pyridine-5-carboxamide; N-methyl-3-(4-(2-(trifluoromethyl)phenyl)piperidine-1-carbonyl)-1,4,5,7-tetrahydro-6H-pyrazolo[3,4-c]pyridine-6-carboxamide; (5-bromo-1H-indazol-3-yl)(4-(2-(trifluoromethyl)phenyl)piperidin-1-yl)methanone; tert-butyl 3-(4-(2-(trifluoromethyl)phenyl)piperidine-1-carbonyl)-1,4,5,7-tetrahydro-6H-pyrazolo[3,4-c]pyridine-6-carboxylate; tert-butyl 3-(4-(2-(trifluoromethyl)phenyl)piperidine-1-carbonyl)-1,4,6,7-tetrahydro-5H-pyrazolo[4,3-c]pyridine-5-carboxylate; (5-fluoro-1-isopropyl-1H-indazol-3-yl)(4-(2-(trifluoromethyl)phenyl)piperidin-1-yl)methanone; (7-fluoro-1H-indazol-3-yl)(4-(2-(trifluoromethyl)phenyl)piperidin-1-yl)methanone; (1H-pyrazolo[4,3-c]pyridin-3-yl)(4-(2-(trifluoromethyl)phenyl)piperidin-1-yl)methanone; (1H-pyrazolo[3,4-c]pyridin-3-yl)(4-(2-(trifluoromethyl)phenyl)piperidin-1-yl)methanone; (1H-pyrazolo[4,3-b]pyridin-3-yl)(4-(2-(trifluoromethyl)phenyl)piperidin-1-yl)methanone; (6-methoxy-1H-indazol-3-yl)(4-(2-(trifluoromethyl)phenyl)piperidin-1-yl)methanone; (5-fluoro-1-(oxetan-3-yl)-1H-indazol-3-yl)(4-(2-(trifluoromethyl)phenyl)piperidin-1-yl)methanone; (1-ethyl-5-(methylsulfonyl)-4,5,6,7-tetrahydro-1H-pyrazolo[4,3-c]pyridin-3-yl)(4-(2-(trifluoromethyl)phenyl)piperidin-1-yl)methanone; (1-ethyl-6-(methylsulfonyl)-4,5,6,7-tetrahydro-1H-pyrazolo[3, 4-c]pyridin-3-yl)(4-(2-(trifluoromethyl)phenyl)piperidin-1-yl)methanone; 1-(1-methyl-3-(4-(2-(trifluoromethyl)phenyl)piperidine-1-carbonyl)-1,4,6,7-tetrahydro-5H-pyrazolo[4,3-c]pyridin-5-yl)ethan-1-one; 1-(1-methyl-3-(4-(2-(trifluoromethyl)phenyl)piperidine-1-carbonyl)-1,4,5,7-tetrahydro-6H-pyrazolo[3,4-c]pyridin-6-yl)ethan-1-one; N,N-dimethyl-3-(4-(2-(trifluoromethyl)phenyl)piperidine-1-carbonyl)-1,4,6,7-tetrahydro-5H-pyrazolo[4,3-c]pyridine-5-carboxamide; N,N-dimethyl-3-(4-(2-(trifluoromethyl)phenyl)piperidine-1-carbonyl)-1,4,5,7-tetrahydro-6H-pyrazolo[3,4-c]pyridine-6-carboxamide; (1-methyl-5,5-dioxido-1,4,6,7-tetrahydrothiopyrano[4,3-c]pyrazol-3-yl)(4-(2-(trifluoromethyl)phenyl)piperidin-1-yl)methanone; (4-(2-(trifluoromethyl)phenyl)piperidin-1-yl)(1, 6,6-trimethyl-1,4,6,7-tetrahydropyrano[4,3-c]pyrazol-3-yl)methanone; (1-methyl-1,4,6,7-tetrahydropyrano[4,3-c]pyrazol-3-yl)(4-(2-(trifluoromethyl)phenyl)piperidin-1-yl)methanone; 2-methyl-1-(3-(4-(2-(trifluoromethyl)phenyl)piperidine-1-carbonyl)-1,4,5,7-tetrahydro-6H-pyrazolo[3,4-c]pyridin-6-yl)propan-1-one; (6-(isopropylsulfonyl)-4,5,6,7-tetrahydro-1H-pyrazolo[3,4-c]pyridin-3-yl)(4-(2-(trifluoromethyl)phenyl)piperidin-1-yl)methanone; (6-(ethylsulfonyl)-4,5,6,7-tetrahydro-1H-pyrazolo[3,4-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,5,7-tetrahydro-6H-pyrazolo[3,4-c]pyridin-6-yl)propan-1-one; 2-methoxy-1-(3-(4-(2-(trifluoromethyl)phenyl)piperidine-1-carbonyl)-1,4,6,7-tetrahy dro-5H-pyrazolo[4,3-c]pyridin-5-yl)ethan-1-one; 3,3,3-trifluoro-1-(3-(4-(2-(trifluoromethyl)phenyl)piperidine-1-carbonyl)-1,4,6,7-tetrahydro-5H-pyrazolo[4,3-c]pyridin-5-yl)propan-1-one; (1H-indazol-3-yl)(4-(2-(trifluoromethyl)phenyl)piperidin-1-yl)methanone; (1-methyl-1H-indazol-3-yl)(4-(2-(trifluoromethyl)phenyl)piperidin-1-yl)methanone; (6-(oxetan-3-yl)-4,5,6,7-tetrahydro-1H-pyrazolo[3,4-c]pyridin-3-yl)(4-(2-(trifluoromethyl)phenyl)piperidin-1-yl)methanone; (6-(tert-butylsulfonyl)-4,5,6,7-tetrahydro-1H-pyrazolo[3,4-c]pyridin-3-yl)(4-(2-(trifluoromethyl)phenyl)piperidin-1-yl)methanone; 2,2-dimethyl-1-(3-(4-(2-(trifluoromethyl)phenyl)piperidine-1-carbonyl)-1,4,5,7-tetrahydro-6H-pyrazolo[3,4-c]pyridin-6-yl)propan-1-one; (6-(tert-butyl)-4,5,6,7-tetrahydro-1H-pyrazolo[3,4-c]pyridin-3-yl)(4-(2-(trifluoromethyl)phenyl)piperidin-1-yl)methanone; (6-(isobutylsulfonyl)-4,5,6,7-tetrahydro-1H-pyrazolo[3,4-c]pyridin-3-yl)(4-(2-(trifluoromethyl)phenyl)piperidin-1-yl)methanone; 3-methyl-1-(3-(4-(2-(trifluoromethyl)phenyl)piperidine-1-carbonyl)-1,4,5,7-tetrahydro-6H-pyrazolo[3,4-c]pyridin-6-yl)butan-1-one; (6-isobutyl-4,5,6,7-tetrahydro-1H-pyrazolo[3,4-c]pyridin-3-yl)(4-(2-(trifluoromethyl)phenyl)piperidin-1-yl)methanone; (6-isopropyl-4,5,6,7-tetrahydro-1H-pyrazolo[3,4-c]pyridin-3-yl)(4-(2-(trifluoromethyl)phenyl)piperidin-1-yl)methanone; (6-ethyl-4,5,6,7-tetrahydro-1H-pyrazolo[3,4-c]pyridin-3-yl)(4-(2-(trifluoromethyl)phenyl)piperidin-1-yl)methanone; (5-(tert-butylsulfonyl)-4,5,6,7-tetrahydro-1H-pyrazolo[4,3-c]pyridin-3-yl)(4-(2-(trifluoromethyl)phenyl)piperidin-1-yl)methanone; tert-butyl 3-(4-fluoro-4-(2-(trifluoromethyl)phenyl)piperidine-1-carbonyl)-1,4,6,7-tetrahydro-5H-pyrazolo[4,3-c]pyridine-5-carboxylate; (4-hydroxy-4-(2-(trifluoromethyl)phenyl)piperidin-1-yl)(1-methyl-IH-indazol-3-yl)methanone; 1-(3-(4-hydroxy-4-(2-(trifluoromethyl)phenyl)piperidine-1-carbonyl)-1,4,6,7-tetrahydro-5H-pyrazolo[4,3-c]pyridin-5-yl)ethan-1-one; 3-(4-(3,4-difluoro-2-(trifluoromethyl)phenyl)piperidine-1-carbonyl)-N-methyl-4,6-dihydropyrrolo[3,4-c]pyrazole-5(1H)-carboxamide; (4-(3,4-difluoro-2-(trifluoromethyl)phenyl)piperidin-1-yl)(5-neopentyl-1,4,5,6-tetrahydropyrrolo[3,4-c]pyrazol-3-yl)methanone; (4-(3,4-difluoro-2-(trifluoromethyl)phenyl)piperidin-1-yl)(5-(oxetan-3-yl)-1,4,5,6-tetrahydropyrrolo[3,4-c]pyrazol-3-yl)methanone; (5-(cy clopropylmethyl)-1,4,5,6-tetrahydropyrrolo[3,4-c]pyrazol-3-yl)(4-(3,4-difluoro-2-(trifluoromethyl)phenyl)piperidin-1-yl)methanone; (4-(3,4-difluoro-2-(trifluoromethyl)phenyl)piperidin-1-yl)(5-ethyl-1,4,5,6-tetrahydropyrrolo[3,4-c]pyrazol-3-yl)methanone; 1-(3-(4-(3,4-difluoro-2-(trifluoromethyl)phenyl)piperidine-1-carbonyl)-4,6-dihydropyrrolo[3,4-c]pyrazol-5(1H)-yl)-3-methylbutan-1-one; 1-(3-(4-(3,4-difluoro-2-(trifluoromethyl)phenyl)piperidine-1-carbonyl)-4,6-dihydropyrrolo[3,4-c]pyrazol-5(1H)-yl)-2-methylpropan-1-one; (4-(3,4-difluoro-2-(trifluoromethyl)phenyl)piperidin-1-yl)(5-picolinoyl-1,4,5,6-tetrahydropyrrolo[3,4-c]pyrazol-3-yl)methanone; 3-(4-(3,4-difluoro-2-(trifluoromethyl)phenyl)piperidine-1-carbonyl)-4,6-dihydropyrrolo[3,4-c]pyrazole-5(1H)-carbonitrile; (4-(3,4-difluoro-2-(trifluoromethyl)phenyl)piperidin-1-yl)(5-(2-methoxyethyl)-1,4,5,6-tetrahydropyrrolo[3,4-c]pyrazol-3-yl)methanone; (4-(3,4-difluoro-2-(trifluoromethyl)phenyl)piperidin-1-yl)(5-(3,3,3-trifluoropropyl)-1,4,5,6-tetrahydropyrrolo[3, 4-c]pyrazol-3-yl)methanone; (5-benzoyl-1,4,5,6-tetrahydropyrrolo[3,4-c]pyrazol-3-yl)(4-(3,4-difluoro-2-(trifluoromethyl)phenyl)piperidin-1-yl)methanone; methyl 3-(4-(3,4-difluoro-2-(trifluoromethyl)phenyl)piperidine-1-carbonyl)-4,6-dihydropyrrolo[3,4-c]pyrazole-5(1H)-carboxylate; (4-(3,4-difluoro-2-(trifluoromethyl)phenyl)piperidin-1-yl)(1,4,5,6-tetrahydropyrrolo[3,4-c]pyrazol-3-yl)methanone; (4-(3,4-difluoro-2-(trifluoromethyl)phenyl)piperidin-1-yl)(5-(2,2,2-trifluoroethyl)-1,4,5,6-tetrahydropyrrolo[3, 4-c]pyrazol-3-yl)methanone; (4-(3,4-difluoro-2-(trifluoromethyl)phenyl)piperidin-1-yl)(5-(pyrrolidine-1-carbonyl)-1,4,5,6-tetrahydropyrrolo[3,4-c]pyrazol-3-yl)methanone; (4-(3,4-difluoro-2-(trifluoromethyl)phenyl)piperidin-1-yl)(5-isonicotinoyl-1,4,5,6-tetrahydropyrrolo[3,4-c]pyrazol-3-yl)methanone; (4-(3,4-difluoro-2-(trifluoromethyl)phenyl)piperidin-1-yl)(5-nicotinoyl-1,4,5,6-tetrahydropyrrolo[3,4-c]pyrazol-3-yl)methanone; (4-(3,4-difluoro-2-(trifluoromethyl)phenyl)piperidin-1-yl)(5-(piperidine-1-carbonyl)-1,4,5,6-tetrahydropyrrolo[3,4-c]pyrazol-3-yl)methanone; (4-(3,4-difluoro-2-(trifluoromethyl)phenyl)piperidin-1-yl)(5-(piperazine-1-carbonyl)-1,4,5,6-tetrahydropyrrolo[3,4-c]pyrazol-3-yl)methanone; 1-(3-(4-(3,4-difluoro-2-(trifluoromethyl)phenyl)piperidine-1-carbonyl)-4,6-dihydropyrrolo[3,4-c]pyrazol-5(1H)-yl)propan-1-one; (5,5-dioxido-4,6-dihydro-1H-thieno[3,4-c]pyrazol-3-yl)(4-(2-(trifluoromethyl)phenyl)piperidin-1-yl)methanone; (4,6-dihydro-1H-furo[3,4-c]pyrazol-3-yl)(4-(2-(trifluoromethyl)phenyl)piperidin-1-yl)methanone; (1-ethyl-5-(methylsulfonyl)-1,4,5,6-tetrahydropyrrolo[3,4-c]pyrazol-3-yl)(4-(2-(trifluoromethyl)phenyl)piperidin-1-yl)methanone; (1-methyl-5-(methylsulfonyl)-1,4,5,6-tetrahydropyrrolo[3,4-c]pyrazol-3-yl)(4-(2-(trifluoromethyl)phenyl)piperidin-1-yl)methanone; (1-methyl-1,4,5,6-tetrahydropyrrolo[3,4-c]pyrazol-3-yl)(4-(2-(trifluoromethyl)phenyl)piperidin-1-yl)methanone; 2-methoxy-1-(3-(4-(2-(trifluoromethyl)phenyl)piperidine-1-carbonyl)-4,6-dihydropyrrolo[3,4-c]pyrazol-5(1H)-yl)ethan-1-one; (5-(2-methoxyethyl)-1,4,5,6-tetrahydropyrrolo[3,4-c]pyrazol-3-yl)(4-(2-(trifluoromethyl)phenyl)piperidin-1-yl)methanone; 3,3,3-trifluoro-1-(3-(4-(2-(trifluoromethyl)phenyl)piperidine-1-carbonyl)-4,6-dihydropyrrolo[3,4-c]pyrazol-5(1H)-yl)propan-1-one; (5-(oxetan-3-yl)-1,4,5,6-tetrahydropyrrolo[3,4-c]pyrazol-3-yl)(4-(2-(trifluoromethyl)phenyl)piperidin-1-yl)methanone; (5-(isobutylsulfonyl)-1,4,5,6-tetrahydropyrrolo[3,4-c]pyrazol-3-yl)(4-(2-(trifluoromethyl)phenyl)piperidin-1-yl)methanone; (5-(isopropylsulfonyl)-1,4,5,6-tetrahydropyrrolo[3,4-c]pyrazol-3-yl)(4-(2-(trifluoromethyl)phenyl)piperidin-1-yl)methanone; (5-(ethylsulfonyl)-1,4,5,6-tetrahydropyrrolo[3,4-c]pyrazol-3-yl)(4-(2-(trifluoromethyl)phenyl)piperidin-1-yl)methanone; tert-butyl 3-(4-(2-(trifluoromethyl)phenyl)piperidine-1-carbonyl)-4,6-dihydropyrrolo[3,4-c]pyrazole-5(1H)-carboxylate; (5-methyl-1,4,5,6-tetrahydropyrrolo[3,4-c]pyrazol-3-yl)(4-(2-(trifluoromethyl)phenyl)piperidin-1-yl)methanone; (5-(methylsulfonyl)-1,4,5,6-tetrahydropyrrolo[3,4-c]pyrazol-3-yl)(4-(2-(trifluoromethyl)phenyl)piperidin-1-yl)methanone; 1-(3-(4-(2-(trifluoromethyl)phenyl)piperidine-1-carbonyl)-4,6-dihydropyrrolo[3,4-c]pyrazol-5(1H)-yl)ethan-1-one; (5-(tert-butylsulfonyl)-1,4,5,6-tetrahydropyrrolo[3,4-c]pyrazol-3-yl)(4-(2-(trifluoromethyl)phenyl)piperidin-1-yl)methanone; (5-(tert-butyl)-1,4,5,6-tetrahydropyrrolo[3,4-c]pyrazol-3-yl)(4-(2-(trifluoromethyl)phenyl)piperidin-1-yl)methanone; (5-isobutyl-1,4,5,6-tetrahydropyrrolo[3,4-c]pyrazol-3-yl)(4-(2-(trifluoromethyl)phenyl)piperidin-1-yl)methanone; (5-isopropyl-1,4,5,6-tetrahydropyrrolo[3,4-c]pyrazol-3-yl)(4-(2-(trifluoromethyl)phenyl)piperidin-1-yl)methanone; (5-ethyl-1,4,5,6-tetrahydropyrrolo[3,4-c]pyrazol-3-yl)(4-(2-(trifluoromethyl)phenyl)piperidin-1-yl)methanone; N-methyl-3-(4-(2-(trifluoromethyl)phenyl)piperidine-1-carbonyl)-4,6-dihydropyrrolo[3,4-c]pyrazole-5(1H)-carboxamide; 1-(1-methyl-3-(4-(2-(trifluoromethyl)phenyl)piperidine-1-carbonyl)-4,6-dihydropyrrolo[3,4-c]pyrazol-5(1H)-yl)ethan-1-one; 2,2-dimethyl-1-(3-(4-(2-(trifluoromethyl)phenyl)piperidine-1-carbonyl)-4,6-dihydropyrrolo[3,4-c]pyrazol-5(1H)-yl)propan-1-one; 3-methyl-1-(3-(4-(2-(trifluoromethyl)phenyl)piperidine-1-carbonyl)-4,6-dihydropyrrolo[3,4-c]pyrazol-5(1H)-yl)butan-1-one; 2-methyl-1-(3-(4-(2-(trifluoromethyl)phenyl)piperidine-1-carbonyl)-4,6-dihydropyrrolo[3,4-c]pyrazol-5(1H)-yl)propan-1-one; 1-(3-(4-(2-(trifluoromethyl)phenyl)piperidine-1-carbonyl)-4,6-dihydropyrrolo[3,4-c]pyrazol-5(1H)-yl)propan-1-one; N,N-dimethyl-3-(4-(2-(trifluoromethyl)phenyl)piperidine-1-carbonyl)-4,6-dihydropyrrolo[3,4-c]pyrazole-5(1H)-carboxamide; (5-(2,2,2-trifluoroethyl)-1,4,5,6-tetrahydropyrrolo[3,4-c]pyrazol-3-yl)(4-(2-(trifluoromethyl)phenyl)piperidin-1-yl)methanone; (5-(methoxymethyl)-1,4,5,6-tetrahydropyrrolo[3,4-c]pyrazol-3-yl)(4-(2-(trifluoromethyl)phenyl)piperidin-1-yl)methanone; (1,4,5,6-tetrahydropyrrolo[3,4-c]pyrazol-3-yl)(4-(2-(trifluoromethyl)phenyl)piperidin-1-yl)methanone; tert-butyl 4-(3-(4-(3,4-difluoro-2-(trifluoromethyl)phenyl)piperidine-1-carbonyl)-1,4,5,6-tetrahydropyrrolo[3,4-c]pyrazole-5-carbonyl)piperazine-1-carboxylate; tert-butyl 3-(4-(3,4-difluoro-2-(trifluoromethyl)phenyl)piperidine-1-carbonyl)-4,6-dihydropyrrolo[3,4-c]pyrazole-5(1H)-carboxylate; (5,5-dioxido-4,6,7,8-tetrahydro-1H-thiepino[4,3-c]pyrazol-3-yl)(4-(2-(trifluoromethyl)phenyl)piperidin-1-yl)methanone; (4,6,7,8-tetrahydro-1H-oxepino[4,3-c]pyrazol-3-yl)(4-(2-(trifluoromethyl)phenyl)piperidin-1-yl)methanone; N-methyl-3-(4-(2-(trifluoromethyl)phenyl)piperidine-1-carbonyl)-4,6,7,8-tetrahydropyrazolo[4,3-c]azepine-5(1H)-carboxamide; (5-(2,2,2-trifluoroethyl)-1,4,5,6,7,8-hexahydropyrazolo[4,3-c]azepin-3-yl)(4-(2-(trifluoromethyl)phenyl)piperidin-1-yl)methanone; (5-(tert-butylsulfonyl)-1,4,5,6,7,8-hexahydropyrazolo[4,3-c]azepin-3-yl)(4-(2-(trifluoromethyl)phenyl)piperidin-1-yl)methanone; 2,2-dimethyl-1-(3-(4-(2-(trifluoromethyl)phenyl)piperidine-1-carbonyl)-4,6,7,8-tetrahydropyrazolo[4,3-c]azepin-5(1H)-yl)propan-1-one; (5-(tert-butyl)-1,4,5,6,7,8-hexahydropyrazolo[4,3-c]azepin-3-yl)(4-(2-(trifluoromethyl)phenyl)piperidin-1-yl)methanone; (5-(isobutylsulfonyl)-1,4,5,6,7,8-hexahydropyrazolo[4,3-c]azepin-3-yl)(4-(2-(trifluoromethyl)phenyl)piperidin-1-yl)methanone; 3-methyl-1-(3-(4-(2-(trifluoromethyl)phenyl)piperidine-1-carbonyl)-4,6,7,8-tetrahydropyrazolo[4,3-c]azepin-5(1H)-yl)butan-1-one; (5-isobutyl-1,4,5,6,7,8-hexahydropyrazolo[4,3-c]azepin-3-yl)(4-(2-(trifluoromethyl)phenyl)piperidin-1-yl)methanone; (5-(isopropylsulfonyl)-1,4,5,6,7,8-hexahydropyrazolo[4,3-c]azepin-3-yl)(4-(2-(trifluoromethyl)phenyl)piperidin-1-yl)methanone; 2-methyl-1-(3-(4-(2-(trifluoromethyl)phenyl)piperidine-1-carbonyl)-4,6,7,8-tetrahydropyrazolo[4,3-c]azepin-5(1H)-yl)propan-1-one; (1-ethyl-5-(methylsulfonyl)-1,4,5,6,7,8-hexahydropyrazolo[4,3-c]azepin-3-yl)(4-(2-(trifluoromethyl)phenyl)piperidin-1-yl)methanone; 1-(1-methyl-3-(4-(2-(trifluoromethyl)phenyl)piperidine-1-carbonyl)-4,6,7,8-tetrahydropyrazolo[4,3-c]azepin-5(1H)-yl)ethan-1-one; (5-(methoxymethyl)-1,4,5,6,7,8-hexahydropyrazolo[4,3-c]azepin-3-yl)(4-(2-(trifluoromethyl)phenyl)piperidin-1-yl)methanone; (5-methyl-1,4,5,6,7,8-hexahydropyrazolo[4,3-c]azepin-3-yl)(4-(2-(trifluoromethyl)phenyl)piperidin-1-yl)methanone; (5-isopropyl-1,4,5,6,7,8-hexahydropyrazolo[4,3-c]azepin-3-yl)(4-(2-(trifluoromethyl)phenyl)piperidin-1-yl)methanone; (5-(ethylsulfonyl)-1,4,5,6,7,8-hexahydropyrazolo[4,3-c]azepin-3-yl)(4-(2-(trifluoromethyl)phenyl)piperidin-1-yl)methanone; 1-(3-(4-(2-(trifluoromethyl)phenyl)piperidine-1-carbonyl)-4,6,7,8-tetrahydropyrazolo[4,3-c]azepin-5(1H)-yl)propan-1-one; (5-ethyl-1,4,5,6,7,8-hexahydropyrazolo[4,3-c]azepin-3-yl)(4-(2-(trifluoromethyl)phenyl)piperidin-1-yl)methanone; (5-(methylsulfonyl)-1,4,5,6,7,8-hexahydropyrazolo[4,3-c]azepin-3-yl)(4-(2-(trifluoromethyl)phenyl)piperidin-1-yl)methanone; 1-(3-(4-(2-(trifluoromethyl)phenyl)piperidine-1-carbonyl)-4,6,7,8-tetrahydropyrazolo[4,3-c]azepin-5(1H)-yl)ethan-1-one; (1,4,5,6,7,8-hexahydropyrazolo[4,3-c]azepin-3-yl)(4-(2-(trifluoromethyl)phenyl)piperidin-1-yl)methanone; (1-methyl-5-(methylsulfonyl)-1, 4,5,6,7,8-hexahydropyrazolo[4,3-c]azepin-3-yl)(4-(2-(trifluoromethyl)phenyl)piperidin-1-yl)methanone; (1-methyl-1,4,5,6,7,8-hexahydropyrazolo[4,3-c]azepin-3-yl)(4-(2-(trifluoromethyl)phenyl)piperidin-1-yl)methanone; N,N-dimethyl-3-(4-(2-(trifluoromethyl)phenyl)piperidine-1-carbonyl)-4,6,7,8-tetrahydropyrazolo[4,3-c]azepine-5(1H)-carboxamide; (5-(2-methoxyethyl)-1,4,5,6,7,8-hexahydropyrazolo[4,3-c]azepin-3-yl)(4-(2-(trifluoromethyl)phenyl)piperidin-1-yl)methanone; 2-methoxy-1-(3-(4-(2-(trifluoromethyl)phenyl)piperidine-1-carbonyl)-4,6,7,8-tetrahydropyrazolo[4,3-c]azepin-5(1H)-yl)ethan-1-one; 3,3,3-trifluoro-1-(3-(4-(2-(trifluoromethyl)phenyl)piperidine-1-carbonyl)-4,6,7,8-tetrahydropyrazolo[4,3-c]azepin-5(1H)-yl)propan-1-one; (5-(oxetan-3-yl)-1,4,5,6,7,8-hexahydropyrazolo[4,3-c]azepin-3-yl)(4-(2-(trifluoromethyl)phenyl)piperidin-1-yl)methanone; tert-butyl 3-(4-(2-(trifluoromethyl)phenyl)piperidine-1-carbonyl)-4,6,7,8-tetrahydropyrazolo[4,3-c]azepine-5(1H)-carboxylate; (6-(trifluoromethyl)imidazo[1,2-b]pyridazin-2-yl)(4-(2-(trifluoromethyl)phenyl)piperidin-1-yl)methanone; (6-fluoroimidazo[1,2-b]pyridazin-2-yl)(4-(2-(trifluoromethyl)phenyl)piperidin-1-yl)methanone; (6-(pyrrolidin-1-yl)imidazo[1,2-b]pyridazin-2-yl)(4-(2-(trifluoromethyl)phenyl)piperidin-1-yl)methanone; (6-cyclopropylimidazo[1,2-b]pyridazin-2-yl)(4-(2-(trifluoromethyl)phenyl)piperidin-1-yl)methanone; (6-methoxyimidazo[1,2-b]pyridazin-2-yl)(4-(2-(trifluoromethyl)phenyl)piperidin-1-yl)methanone; (6-methylimidazo[1,2-b]pyridazin-2-yl)(4-(2-(trifluoromethyl)phenyl)piperidin-1-yl)methanone; (6-chloroimidazo[1,2-b]pyridazin-2-yl)(4-(2-(trifluoromethyl)phenyl)piperidin-1-yl)methanone; imidazo[1,2-b]pyridazin-2-yl(4-(2-(trifluoromethyl)phenyl)piperidin-1-yl)methanone; (6-chloro-2-methylimidazo[1,2-b]pyridazin-3-yl)(4-(2-(trifluoromethyl)phenyl)piperidin-1-yl)methanone; (1H-benzo[d]imidazol-2-yl)(4-(2-(trifluoromethyl)phenyl)piperidin-1-yl)methanone; (1H-imidazo[4,5-b]pyridin-2-yl)(4-(2-(trifluoromethyl)phenyl)piperidin-1-yl)methanone; (4-(5-fluoro-2-(trifluoromethyl)phenyl)piperidin-1-yl)(4,5,6,7-tetrahydro-1H-pyrazolo[3,4-c]pyridin-3-yl)methanone; (4-(3-fluoro-2-(trifluoromethyl)phenyl)piperidin-1-yl)(6-(2-methoxyethyl)-4,5, 6,7-tetrahydro-1H-pyrazolo[3,4-c]pyridin-3-yl)methanone; 6-methyl-2-(4-(2-(trifluoromethyl)phenyl)piperidine-1-carbonyl)pyrimidine-4-carboxylic acid; methyl 6-methyl-2-(4-(2-(trifluoromethyl)phenyl)piperidine-1-carbonyl)pyrimidine-4-carboxylate; N-(cyclopropylsulfonyl)-2-(4-(2-(trifluoromethyl)phenyl)piperidine-1-carbonyl)benzamide; N-(phenylsulfonyl)-2-(4-(2-(trifluoromethyl)phenyl)piperidine-1-carbonyl)benzamide; N-(methylsulfonyl)-2-(4-(2-(trifluoromethyl)phenyl)piperidine-1-carbonyl)benzamide; 3-(4-(2-(trifluoromethyl)phenyl)piperidine-1-carbonyl)benzamide; 2-(4-(2-(trifluoromethyl)phenyl)piperidine-1-carbonyl)benzamide; 4-(4-(2-(trifluoromethyl)phenyl)piperidine-1-carbonyl)benzoic acid; 3-(4-(2-(trifluoromethyl)phenyl)piperidine-1-carbonyl)benzoic acid; 2-(4-(2-(trifluoromethyl)phenyl)piperidine-1-carbonyl)benzoic acid; 4-(4-(2-(tert-butyl)phenyl)piperidine-1-carbonyl)benzoic acid; 2-(4-(2-(tert-butyl)phenyl)piperidine-1-carbonyl)benzoic acid; 3-(4-(2-(tert-butyl)phenyl)piperidine-1-carbonyl)benzoic acid; 4-(4-(2-(trifluoromethyl)phenyl)piperidine-1-carbonyl)benzamide; 1-(3-(4-(2-(trifluoromethyl)phenyl)piperidine-1-carbonyl)-4,7-dihydroisothiazolo[5,4-c]pyridin-6(5H)-yl)ethan-1-one; (4,5,6,7-tetrahydroisothiazolo[5,4-c]pyridin-3-yl)(4-(2-(trifluoromethyl)phenyl)piperidin-1-yl)methanone; (4,5,6,7-tetrahydroisothiazolo[4,5-c]pyridin-3-yl)(4-(2-(trifluoromethyl)phenyl)piperidin-1-yl)methanone; 1-(3-(4-(2-(trifluoromethyl)phenyl)piperidine-1-carbonyl)-6,7-dihydroisoxazolo[4,5-c]pyridin-5(4H)-yl)ethan-1-one; 1-(3-(4-(2-(trifluoromethyl)phenyl)piperidine-1-carbonyl)-4,7-dihydroisoxazolo[5,4-c]pyridin-6(5H)-yl)ethan-1-one; (4,5,6,7-tetrahydroisoxazolo[4,5-c]pyridin-3-yl)(4-(2-(trifluoromethyl)phenyl)piperidin-1-yl)methanone; benzo[c]isothiazol-3-yl(4-(2-(trifluoromethyl)phenyl)piperidin-1-yl)methanone; benzo[d]thiazol-2-yl(4-(2-(trifluoromethyl)phenyl)piperidin-1-yl)methanone; benzo[d]isoxazol-3-yl(4-(2-(trifluoromethyl)phenyl)piperidin-1-yl)methanone; 1-(3-(4-(2-(trifluoromethyl)phenyl)piperidine-1-carbonyl)-6,7-dihydroisothiazolo[4,5-c]pyridin-5(4H)-yl)ethan-1-one; benzo[d]oxazol-2-yl(4-(2-(trifluoromethyl)phenyl)piperidin-1-yl)methanone; (3-methyloxetan-3-yl)(4-(2-(trifluoromethyl)phenyl)piperidin-1-yl)methanone; oxetan-3-yl(4-(2-(trifluoromethyl)phenyl)piperidin-1-yl)methanone; 2-(2-hydroxyphenyl)-1-(4-(2-(trifluoromethyl)phenyl)piperidin-1-yl)ethan-1-one; (4-(2-(tert-butyl)phenyl)piperidin-1-yl)(tetrahydrothiophen-2-yl)methanone; rac-tert-butyl (2R,3R)-2-(4-(2-(tert-butyl)phenyl)piperidine-1-carbonyl)-3-hydroxypyrrolidine-1-carboxylate; rac-tert-butyl (2R,4R)-2-(4-(2-(tert-butyl)phenyl)piperidine-1-carbonyl)-4-hydroxypyrrolidine-1-carboxylate2-(2-oxo-2-(4-(2-(trifluoromethyl)phenyl)piperidin-1-yl)ethyl)phenyl sulfamate; (4-(2-(tert-butyl)phenyl)piperidin-1-yl)(1,1-dioxidotetrahydrothiophen-2-yl)methanone; rac-(4-(2-(tert-butyl)phenyl)piperidin-1-yl)((2R,3R)-3-hydroxypyrrolidin-2-yl)methanone; rac-(4-(2-(tert-butyl)phenyl)piperidin-1-yl)((2R,4R)-4-hydroxypyrrolidin-2-yl)methanone; rac-(R)-1-(2-(4-(2-(tert-butyl)phenyl)piperidine-1-carbonyl)pyrrolidin-1-yl)ethan-1-one; (6-bromo-1H-pyrrolo[3,2-b]pyridin-2-yl)(4-(2-(trifluoromethyl)phenyl)piperidin-1-yl)methanone; (5-morpholino-1H-pyrrolo[3,2-b]pyridin-2-yl)(4-(2-(trifluoromethyl)phenyl)piperidin-1-yl)methanone; (5-(1H-imidazol-1-yl)-1H-pyrrolo[3,2-b]pyridin-2-yl)(4-(2-(trifluoromethyl)phenyl)piperidin-1-yl)methanone; (5-chloro-1H-pyrrolo[3,2-b]pyridin-2-yl)(4-(2-(trifluoromethyl)phenyl)piperidin-1-yl)methanone; (5-fluoro-1H-pyrrolo[3,2-b]pyridin-2-yl)(4-(2-(trifluoromethyl)phenyl)piperidin-1-yl)methanone; (5-methyl-iH-pyrrolo[3,2-b]pyridin-2-yl)(4-(2-(trifluoromethyl)phenyl)piperidin-1-yl)methanone; (6-methoxy-1H-pyrrolo[3,2-b]pyridin-2-yl)(4-(2-(trifluoromethyl)phenyl)piperidin-1-yl)methanone; imidazo[1,2-a]pyridin-2-yl(4-(2-(trifluoromethyl)phenyl)piperidin-1-yl)methanone; (6-chloro-2-methylimidazo[1,2-b]pyridazin-3-yl)(4-(2-(trifluoromethyl)phenyl)piperidin-1-yl)methanone; imidazo[1,2-b]pyridazin-6-yl(4-(2-(trifluoromethyl)phenyl)piperidin-1-yl)methanone; (1H-pyrrolo[2,3-b]pyridin-2-yl)(4-(2-(trifluoromethyl)phenyl)piperidin-1-yl)methanone; (1H-pyrrolo[3,2-c]pyridin-2-yl)(4-(2-(trifluoromethyl)phenyl)piperidin-1-yl)methanone; (6-chloro-1H-pyrrolo[3,2-b]pyridin-2-yl)(4-(2-(trifluoromethyl)phenyl)piperidin-1-yl)methanone; (6-morpholino-1H-pyrrolo[3,2-b]pyridin-2-yl)(4-(2-(trifluoromethyl)phenyl)piperidin-1-yl)methanone; (6-(1H-imidazol-1-yl)-1H-pyrrolo[3,2-b]pyridin-2-yl)(4-(2-(trifluoromethyl)phenyl)piperidin-1-yl)methanone; (1-methyl-iH-pyrrolo[3,2-b]pyridin-2-yl)(4-(2-(trifluoromethyl)phenyl)piperidin-1-yl)methanone; (5-methoxy-1H-pyrrolo[3,2-b]pyridin-2-yl)(4-(2-(trifluoromethyl)phenyl)piperidin-1-yl)methanone; (6-fluoro-1H-pyrrolo[3,2-b]pyridin-2-yl)(4-(2-(trifluoromethyl)phenyl)piperidin-1-yl)methanone; (1H-imidazo[4,5-b]pyridin-2-yl)(4-(2-(trifluoromethyl)phenyl)piperidin-1-yl)methanone; (6-methyl-1H-pyrrolo[3,2-b]pyridin-2-yl)(4-(2-(trifluoromethyl)phenyl)piperidin-1-yl)methanone; (1H-indol-2-yl)(4-(2-(trifluoromethyl)phenyl)piperidin-1-yl)methanone; (1H-pyrrolo[3,2-b]pyridin-2-yl)(4-(2-(trifluoromethyl)phenyl)piperidin-1-yl)methanone; (1H-pyrrolo[2,3-c]pyridin-2-yl)(4-(2-(trifluoromethyl)phenyl)piperidin-1-yl)methanone; (1H-pyrazol-3-yl)(4-(2-(trifluoromethyl)phenyl)piperidin-1-yl)methanone; (1H-1,2,3-triazol-5-yl)(4-(2-(trifluoromethyl)phenyl)piperidin-1-yl)methanone; pyrazin-2-yl(4-(2-(trifluoromethyl)phenyl)piperidin-1-yl)methanone; (6-methoxypyridazin-3-yl)(4-(2-(trifluoromethyl)phenyl)piperidin-1-yl)methanone; (6-methylpyridazin-3-yl)(4-(2-(trifluoromethyl)phenyl)piperidin-1-yl)methanone; (4-methyl-1,2,3-thiadiazol-5-yl)(4-(2-(trifluoromethyl)phenyl)piperidin-1-yl)methanone; (6-chloropyridazin-3-yl)(4-(2-(trifluoromethyl)phenyl)piperidin-1-yl)methanone; pyridazin-3-yl(4-(2-(trifluoromethyl)phenyl)piperidin-1-yl)methanone; pyridazin-4-yl(4-(2-(trifluoromethyl)phenyl)piperidin-1-yl)methanone; 4-(2-(trifluoromethyl)phenyl)piperidine-1-carboxylic acid; or 3-oxo-3-(4-(2-(trifluoromethyl)phenyl)piperidin-1-yl)propanoic acid.

Some embodiments provided herein describe an RBP4 inhibitor, or a pharmaceutically acceptable salt, solvate, polymorph, prodrug, metabolite, N-oxide, stereoisomer, or isomer thereof, for use in treating age-related macular degeneration, having the structure of Formula (II):

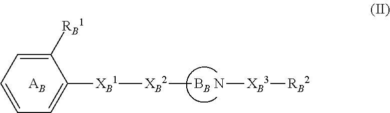

wherein;
   ring A_{B} is benzene optionally further substituted;
   R_{B}¹ is an optionally substituted branched C₃₋₆ alkyl group;
   X_{B}¹ is O, S, SO, SO₂, or NH;
   X_{B}² is a bond or a C₁-C₃ alkylene group;
   ring B_{B} is azetidine, pyrrolidine, or piperidine;
   X_{B}³ is CO or SO₂;
   R_{B}² is a substituent; or
   a pharmaceutically acceptable salt thereof.

In some embodiments, the compound of Formula (II) has the structure

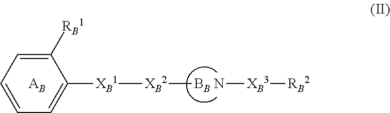

wherein;
   ring A_{B} is benzene optionally further substituted;
   R_{B}¹ is an optionally substituted branched C₃-C₆ alkyl group
   X_{B}¹ is O, S, SO, SO₂, or NH;
   X_{B}² is a bond or a C₁-C₃ alkylene group;
   ring B_{B} is azetidine or piperidine;
   X_{B}³ is CO or SO₂;
   R_{B}² is an optionally substituted hydrocarbon group, an optionally substituted heterocyclic group, an optionally substituted hydroxy group, an optionally substituted mercapto group, a cyano group, a nitro group, an acyl group, or a halogen atom; or
   a pharmaceutically acceptable salt thereof.

In some embodiments, the compound of Formula (II) is 4-(3-(2-tert-butylphenoxy)azetidin-1-yl)-4-oxobutanoic acid, 3-{3-[(2-tert-butyl-4-fluorophenoxy)methyl]azetidin-1-yl}-3-oxopropanoic acid, 2-{[3-(2-tert-butyl-4-chlorophenoxy)azetidin-1-yl]carbonyl}pyridine, 4-[3-(2-tert-butyl-4-chlorophenoxy)azetidin-1-yl]-4-oxobutanoic acid, {3-[(2-tert-butyl-4-chlorophenoxy)methyl]azetidin-1-yl}(oxo)acetic acid, {3-[(2-tert-butylphenoxy)methyl]azetidin-1-yl}(oxo)acetic acid, 3-{3-[(2-tert-butylphenoxy)methyl]azetidin-1-yl}-3-oxopropanoic acid, {4-[(2-tert-butyl-4-chlorophenoxy)methyl]piperidin-1-yl}(oxo)acetic acid, [4-(2-tert-butylphenoxy)piperidin-1-yl](oxo)acetic acid, or {4-[(2-tert-butylphenoxy)methyl]piperidin-1-yl}(oxo)acetic acid, or a pharmaceutically acceptable salt thereof.

Some embodiments provided herein describe an RBP4 inhibitor, or a pharmaceutically acceptable salt, solvate, polymorph, prodrug, metabolite, N-oxide, stereoisomer, or isomer thereof, for use in treating age-related macular degeneration, having the structure of Formula (III):

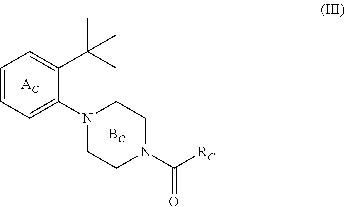

wherein;
   ring A_{C} is a benzene ring optionally further substituted;
   ring B_{C} is a piperazine ring optionally further substituted; and
   R_{C} is a substituent; or
   a pharmaceutically acceptable salt thereof.

In some embodiments, the compound of Formula (III) has the structure

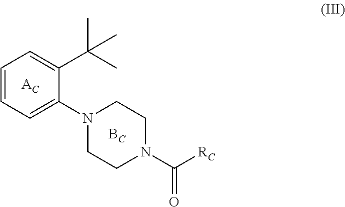

wherein;
   ring A_{C} is a benzene ring optionally substituted by 1 to 3 substituents selected from the group consisting of (a) a halogen atom, and (b) a C₁₋₆ alkyl group;
   ring B_{C} is a piperazine ring optionally substituted by 1 to 3 substituents selected from the group consisting of (a) a halogen atom, (b) a C₁-C₆ alkyl group optionally substituted by 1 to 3 halogen atoms, and (c) a C₁-C₆alkoxy group optionally substituted by 1 to 3 halogen atoms; and
   R_{C} is (1) an optionally substituted C₁-C₁₀ alkyl group, (2) an optionally substituted C₆-C₁₄ aryl group, (3) an optionally substituted 5- or 6-membered aromatic heterocyclic group, (4) an optionally substituted amino group, (5) an optionally substituted carboxy group, or (6) an optionally substituted carbamoyl group; or
   a pharmaceutically acceptable salt thereof.

In some embodiments, the compound of Formula (III) is N-{[4-(2-tert-butylphenyl)piperazin-1-yl]carbonyl}glycine, 3-[4-(2-tert-butylphenyl)piperazin-1-yl]-3-oxopropanoic acid, [4-(2-tert-butyl-4-chlorophenyl)piperazin-1-yl](oxo)acetic acid, 5-{2-[4-(2-tert-butylphenyl)piperazin-1-yl]-2-oxoethyl}imidazolidine-2,4-dione, [(5-{[4-(2-tert-butylphenyl)piperazin-1-yl]carbonyl}isoxazol-3-yl)oxy]acetic acid, or a pharmaceutically acceptable salt thereof.

Some embodiments provided herein describe an RBP4 inhibitor, or a pharmaceutically acceptable salt, solvate, polymorph, prodrug, metabolite, N-oxide, stereoisomer, or isomer thereof, for use in treating age-related macular degeneration, having the structure of Formula (IV):

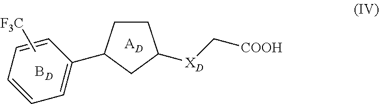

wherein;
   ring A_{D} is a 5-membered non-aromatic heterocycle optionally further substituted by one substituent;
   ring B_{D} is an optionally further substituted benzene ring; and
   X_{D} is a bond, O, CH₂O, OCH₂, CH₂, (CH₂)₂, S, CH₂S, SCH₂, S(O), CH₂S(O), S(O)CH₂, S(O)₂, CH₂S(O)₂, or S(O)₂CH₂; or
   a pharmaceutically acceptable salt thereof.

In some embodiments, the compound of Formula (IV) has the structure

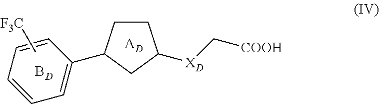

wherein;
   ring A_{D} is a 5-membered non-aromatic heterocycle optionally further substituted by one oxo group;
   ring B_{D} is a benzene ring optionally further substituted by 1 to 4 substituents; and
   X_{D} is O, CH₂O, OCH₂, CH₂, (CH₂)₂, S, CH₂S, SCH₂, S(O), CH₂S(O), S(O)CH₂, S(O)₂, CH₂S(O)₂, or S(O)₂CH₂; or
   a pharmaceutically acceptable salt thereof.

In some embodiments, the compound of Formula (IV) is ({(3S)-1-[3,5-bis(trifluoromethyl)phenyl]pyrrolidin-3-yl}oxy)acetic, ({1-[4-chloro-3-(trifluoromethyl)phenyl]pyrrolidin-3-yl}sulfanyl)acetic acid, 3-{(2R,5S)-5-[3,5-bis(trifluoromethyl)phenyl]tetrahydrofuran-2-yl}propanoic acid, or a pharmaceutically acceptable salt thereof.

Some embodiments provided herein describe an RBP4 inhibitor, or a pharmaceutically acceptable salt, solvate, polymorph, prodrug, metabolite, N-oxide, stereoisomer, or isomer thereof, for use in treating age-related macular degeneration, having the structure of Formula (V):

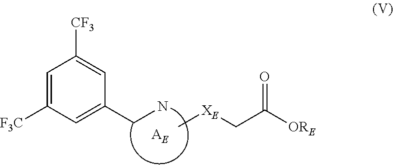

wherein;
   ring A_{E} is an optionally further substituted monocyclic nitrogen-containing aromatic heterocycle;
   X_{E} is CH₂ or O; and
   R_{E} is a hydrogen atom or a C₁-C₆ alkyl group; or
   a pharmaceutically acceptable salt thereof.

In some embodiments, the compound of Formula (V) has the structure

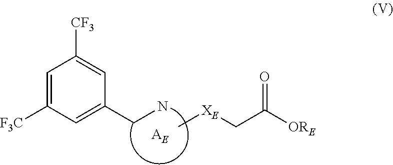

wherein;
   ring A_{E} is a pyrazole ring, a pyridine ring, an oxazole ring, an imidazole ring, or a pyrimidine ring;
   X_{E} is S, optionally substituted alkylene, or O; and
   R_{E} is a hydrogen atom or a C₁-C₆ alkyl group; or
   a pharmaceutically acceptable salt thereof.

In some embodiments, the compound of Formula (V) is ((4-(3,5-bis(trifluoromethyl)phenyl)-1,3-oxazol-2-yl)sulfanyl)acetic acid, ethyl ((6-(3,5-bis(trifluoromethyl)phenyl)-pyridin-3-yl)sulfanyl)acetic acid, ((6-(3,5-bis(trifluoromethyl)-phenyl)pyridine-3-yl)sulfanyl)acetic acid, or 3-(3-(3,5-bis(trifluoromethyl)phenyl)-1H-pyrazol-1-yl)butanoic acid.

In some embodiments, the RBP4 inhibitor is an RBP4 inhibitor provided in Table B.

TABLE B
RBP4 Inhibitors

| Com- | | |
|---|---|---|
| pound | | |
| No. | Name | Structure |
| 1 | 1-(3-(4-(3,4-difluoro-2- (trifluoromethyl)phenyl)piperidine-1- carbonyl)-1,4,5,7-tetrahydro-6H- pyrazolo[3,4-c]pyridin-6-yl)ethan-1-one | 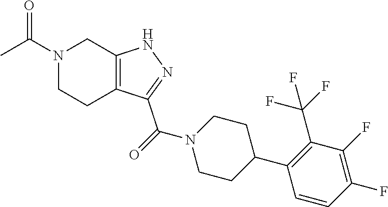 [SMILES: CC(=O)N1CCc2c(C(=O)N3CCC(c4ccc(F)c(F)c4C(F)(F)F)CC3)n[nH]c2C1] |
| 2 | 2-(3-((2-(tert-butyl)-4- chlorophenoxy)methyl)azetidin-1-yl)-2- oxoacetic acid | 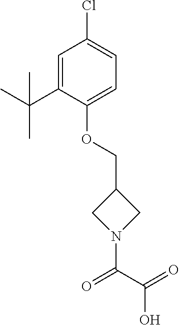 [SMILES: CC(C)(C)c1cc(Cl)ccc1OCC1CN(C(=O)C(=O)O)C1] |
| 3 | 3-(3-(2-(tert-butyl)-4-fluorophenoxy)azetidin- 1-yl)-3-oxopropanoic acid | 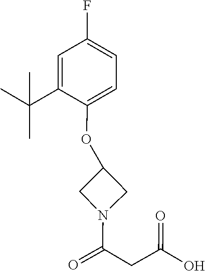 [SMILES: CC(C)(C)c1cc(F)ccc1OC1CN(C(=O)CC(=O)O)C1] |
| 4 | 4-(3-(2-(tert-butyl)-4-fluorophenoxy)azetidin- 1-yl)-4-oxobutanoic acid | 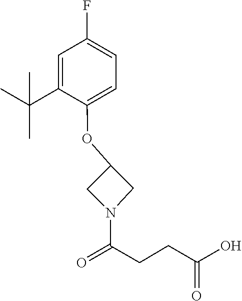 [SMILES: CC(C)(C)c1cc(F)ccc1OC1CN(C(=O)CCC(=O)O)C1] |
| 5 | (4-(2-(tert-butyl)phenoxy)piperidin-1- yl)(pyridin-2-yl)methanone | 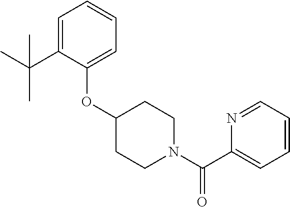 [SMILES: CC(C)(C)c1ccccc1OC1CCN(C(=O)c2ccccn2)CC1] |
| 6 | 1-(3-(2-(tert-butyl)phenoxy)azetidine-1- carbonyl)cyclopropane-1-carboxylic acid | 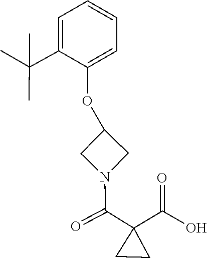 [SMILES: CC(C)(C)c1ccccc1OC1CN(C(=O)C2(C(=O)O)CC2)C1] |
| 7 | (3-(2-(tert-butyl)-4-fluorophenoxy)azetidin-1- yl)(pyridin-2-yl)methanone | 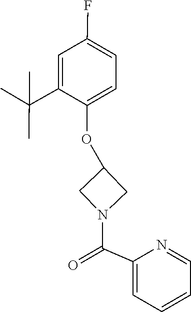 [SMILES: CC(C)(C)c1cc(F)ccc1OC1CN(C(=O)c2ccccn2)C1] |
| 8 | 3-(4-(2-(tert-butyl)phenyl)piperazin-1-yl)-3- oxopropanoic acid | 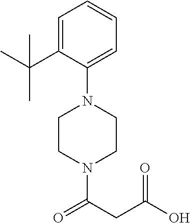 [SMILES: CC(C)(C)c1ccccc1N1CCN(C(=O)CC(=O)O)CC1] |
| 9 | 3-(4-(4-bromo-2-(tert-butyl)phenyl)piperazin- 1-yl)-3-oxopropanoic acid | 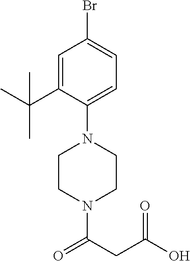 [SMILES: CC(C)(C)c1cc(Br)ccc1N1CCN(C(=O)CC(=O)O)CC1] |
| 10 | 2-(4-(2-(tert-butyl)phenyl)piperazin-1-yl)-2- oxoacetic acid | 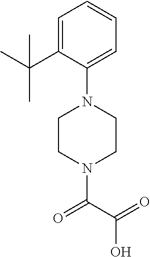 [SMILES: CC(C)(C)c1ccccc1N1CCN(C(=O)C(=O)O)CC1] |
| 11 | 4-(2-(tert-butyl)phenyl)-N-phenylpiperazine- 1-carboxamide | 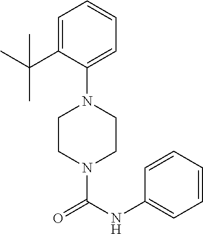 [SMILES: CC(C)(C)c1ccccc1N1CCN(C(=O)Nc2ccccc2)CC1] |
| 12 | (4-(2-(tert-butyl)phenyl)piperazin-1-yl)(1H- imidazol-4-yl)methanone | 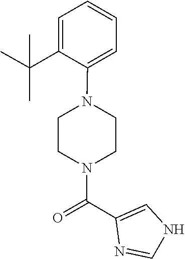 [SMILES: CC(C)(C)c1ccccc1N1CCN(C(=O)c2c[nH]cn2)CC1] |
| 13 | 2-((1-(3,5- bis(trifluoromethyl)phenyl)pyrrolidin-3- yl)thio)acetic acid | 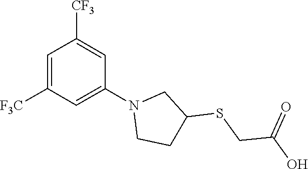 [SMILES: O=C(O)CSC1CCN(c2cc(C(F)(F)F)cc(C(F)(F)F)c2)C1] |
| 14 | 2-((1-(3,5- bis(trifluoromethyl)phenyl)pyrrolidin-3- yl)oxy)acetic acid | 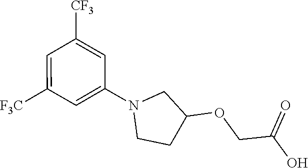 [SMILES: O=C(O)COC1CCN(c2cc(C(F)(F)F)cc(C(F)(F)F)c2)C1] |
| 15 | 3-(5-(3,5- bis(trifluoromethyl)phenyl)tetrahydrofuran-2- yl)propanoic acid | 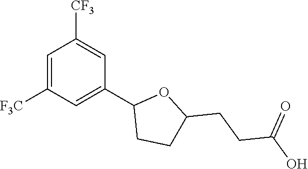 [SMILES: O=C(O)CCC1CCC(c2cc(C(F)(F)F)cc(C(F)(F)F)c2)O1] |
| 16 | 2-((4-(3,5-bis(trifluoromethyl)phenyl)oxazol- 2-yl)thio)acetic acid | 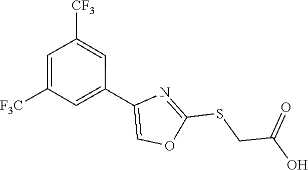 [SMILES: O=C(O)CSc1nc(-c2cc(C(F)(F)F)cc(C(F)(F)F)c2)co1] |
| 17 | 2-((6-(3,5-bis(trifluoromethyl)phenyl)pyridin- 3-yl)thio)acetic acid | 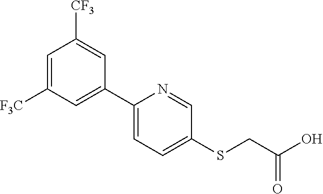 [SMILES: O=C(O)CSc1ccc(-c2cc(C(F)(F)F)cc(C(F)(F)F)c2)nc1] |
| 18 | ethyl 2-((6-(3,5- bis(trifluoromethyl)phenyl)pyridin-3- yl)thio)acetate | 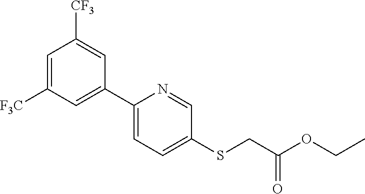 [SMILES: CCOC(=O)CSc1ccc(-c2cc(C(F)(F)F)cc(C(F)(F)F)c2)nc1] |
| 19 | 3-(3-(3,5-bis(trifluoromethyl)phenyl)-1H- pyrazol-1-yl)butanoic acid | 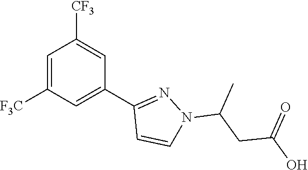 [SMILES: CC(CC(=O)O)n1ccc(-c2cc(C(F)(F)F)cc(C(F)(F)F)c2)n1] |
| 20 | 3-(4-(3-fluoro-2- (trifluoromethyl)phenyl)piperidine-1- carbonyl)-[1,2,4]triazolo[4,3-a]pyridine-6- carbonitrile | 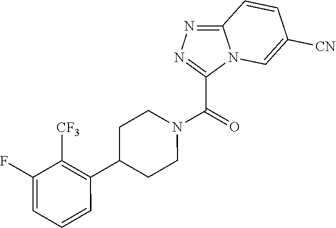 [SMILES: N#Cc1ccc2nnc(C(=O)N3CCC(c4cccc(F)c4C(F)(F)F)CC3)n2c1] |

### Preparation of Compounds

The compounds used in the chemical reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (CostaMesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Alternatively, specific and analogous reactants can be identified through the indices of known chemicals and reactions prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the heterocyclic RBP4 inhibitory compound described herein is P. H. Stahl & C. G. Wermuth “Handbook of Pharmaceutical Salts”, Verlag Helvetica Chimica Acta, Zurich, 2002.

### Retinol Binding Protein 4 (RBP4)

Retinol-binding protein 4 (RBP4), the sole retinol transporter in blood, is secreted from adipocytes and the liver. Lowering levels of RBP4 can lead to reduction in the accumulation of lipofuscin that leads to vision loss in diseases like STGD, AMD, dry (atrophic) Age-Related Macular Degeneration. In some instances, lowering RBP4 reduces the accumulation of lipofuscin in the retina. In some embodiments, compounds and formulations described herein lower serum or plasma RBP4 and thus delay or stop vision loss from excessive accumulation of lipofuscin in the retina. In some embodiments, compounds and formulations described herein lower serum or plasma RBP4 and thus delay or stop vision loss from Age-Related Macular Degeneration.

In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 30% from baseline. In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 30% from baseline. In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 850% from baseline.

In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 30% from baseline. In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 30% from baseline. In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 20% from baseline. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 25% from baseline. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 30% from baseline. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 15 mg/dL.

### Methods of Treatment

In some embodiments, a compound disclosed herein is used to treat or ameliorate a disease associated with altered RBP4 pathways, such as AMD or STGD, when administered to a subject in need thereof. In some cases, a compound disclosed herein is used to treat or ameliorate the effects of a disease associated with altered RBP4 pathway, such as Age-Related Macular Degeneration or STGD, when administered to a subject in need thereof.

### Age-Related Macular Degeneration

Age-related macular degeneration (AMD) is a common eye condition and a leading cause of vision loss among people age 50 and older. It causes damage to the macula, a small spot near the center of the retina and the part of the eye needed for sharp, central vision. As AMD progresses, a blurred area near the center of vision is a common symptom. Overtime, the blurred area may grow larger and the subject may develop blank spots in his or her central vision.

Some embodiments provided herein describe the use of the RBP4 inhibitors described herein for treating AMD in a subject in need thereof. In some embodiments, the RBP4 inhibitors inhibit AMD. In certain embodiments, the RBP4 inhibitors arrest development of AMD or its clinical symptoms. In certain embodiments, the RBP4 inhibitors reduce development of AMD or its clinical symptoms. In certain embodiments, the RBP4 inhibitors relieve the subject of AMD. In certain embodiments, the RBP4 inhibitors cause regression, reversal, or amelioration of AMD. In certain embodiments, the RBP4 inhibitors reduce the number, frequency, duration, or severity of AMD clinical symptoms.

In some embodiments, the RBP4 inhibitors are used prophylactically. In certain embodiments, the RBP4 inhibitors are used to prevent or reduce the risk of developing AMD. In certain embodiments, the RBP4 inhibitors cause the clinical symptoms of AMD to not develop in a subject who may be predisposed to AMD but who does not yet experience or display symptoms of AMD.

### Dry (Atrophic) Age-Related Macular Degeneration

Approximately 85% to 90% of the cases of macular degeneration are the “dry” (atrophic) type. It is estimated that 62.9 million individuals worldwide have this form of AMD; 8 million of them are Americans. Due to increasing life expectancy and current demographics this number is expected to triple by 2020. There is currently no FDA-approved treatment for dry AMD. Given the lack of treatment and high prevalence, development of drugs for dry AMD is of upmost importance. Clinically, atrophic AMD represents a slowly progressing neurodegenerative disorder in which specialized neurons (rod and cone photoreceptors) die in the central part of the retina called the macula. Histopathological and clinical imaging studies indicate that photoreceptor degeneration in dry AMD is triggered by abnormalities in the retinal pigment epithelium (RPE) that lies beneath photoreceptors and provides critical metabolic support to these light-sensing neuronal cells. Experimental and clinical data indicate that excessive accumulation of cytotoxic autofluorescent lipid-protein-retinoid aggregates (lipofuscin) in the RPE is a major trigger of dry AMD. The major cytotoxic component of RPE lipofuscin is pyridinium bisretinoid A2E. Additional cytotoxic bisretinoids are isoA2E, atRAL di-PE, and A2-DHP-PE. Formation of A2E and other lipofuscin bisretinoids, such as A2-DHP-PE (A2-dihydropyridine-phosphatidylethanolamine) and atRALdi-PE (all-trans-retinal dimer-phosphatidylethanolamine), begins in photoreceptor cells in a non-enzymatic manner and can be considered as a by-product of the properly functioning visual cycle.

Some embodiments provided herein describe the use of the RBP4 inhibitors described herein for treating dry (atrophic) AMD in a subject in need thereof. In some embodiments, the RBP4 inhibitors inhibit dry (atrophic) AMD. In certain embodiments, the RBP4 inhibitors arrest development of dry (atrophic) AMD or its clinical symptoms. In certain embodiments, the RBP4 inhibitors reduce development of dry (atrophic) AMD or its clinical symptoms. In certain embodiments, the RBP4 inhibitors relieve the subject of dry (atrophic) AMD. In certain embodiments, the RBP4 inhibitors cause regression, reversal, or amelioration of dry (atrophic) AMD. In certain embodiments, the RBP4 inhibitors reduce the number, frequency, duration, or severity of dry (atrophic) AMD clinical symptoms.

In some embodiments, the RBP4 inhibitors are used prophylactically. In certain embodiments, the RBP4 inhibitors are used to prevent or reduce the risk of developing dry (atrophic) AMD. In certain embodiments, the RBP4 inhibitors cause the clinical symptoms of dry (atrophic) AMD to not develop in a subject who may be predisposed to dry (atrophic) AMD but who does not yet experience or display symptoms of dry (atrophic) AMD.

### Pharmaceutical Compositions

In certain embodiments, the RBP4 inhibitory compound as described herein is administered as a pure chemical. In other embodiments, the RBP4 inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one RBP4 inhibitory compound, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

One embodiment provides a pharmaceutical composition comprising an RBP4 inhibitor, or a pharmaceutically acceptable salt thereof, and a pharmaceutically acceptable excipient. In one embodiment, the pharmaceutical compositions are provided in a dosage form for oral administration, which comprise a compound provided herein, and one or more pharmaceutically acceptable excipients or carriers.

In certain embodiments, the RBP4 inhibitory compound is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract.

In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the pharmaceutical compositions provided herein are formulated for oral administration in tablet, capsule, powder, or liquid form. In some embodiments, a tablet comprises a solid carrier or an adjuvant. Liquid pharmaceutical compositions generally comprise a liquid carrier such as water, petroleum, animal or vegetable oils, mineral oil, or synthetic oil. In some embodiments, physiological saline solution, dextrose or other saccharide solution, or glycols are optionally included. In some embodiments, a capsule comprises a solid carrier such as gelatin.

In another embodiment, the pharmaceutical compositions are provided in a dosage form for parenteral administration, which comprise a compound provided herein, and one or more pharmaceutically acceptable excipients or carriers. Where pharmaceutical compositions are formulated for intravenous, cutaneous or subcutaneous injection, the active ingredient is in the form of a parenterally acceptable aqueous solution, which is pyrogen-free and has a suitable pH, isotonicity, and stability. Those of relevant skill in the art are well able to prepare suitable solutions using, for example, isotonic vehicles, such as Sodium Chloride injection, Ringer's injection, or Lactated Ringer's injection. In some embodiments, preservatives, stabilizers, buffers, antioxidants, and/or other additives are included.

In yet another embodiment, the pharmaceutical compositions are provided in a dosage form for topical administration, which comprise a compound provided herein, and one or more pharmaceutically acceptable excipients or carriers.

### Methods of Dosing and Treatment Regimens

The dose of the composition comprising at least one RBP4 inhibitory compound as described herein differ, depending upon the patient's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

In one embodiment, the compounds described herein, or a pharmaceutically acceptable salt thereof, are used in the preparation of medicaments for the treatment of diseases or conditions in a mammal that would benefit from administration of any one of the compounds disclosed. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, in which the mammal previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Oral doses typically range from about 1.0 mg to about 1000 mg, one to four times, or more, per day. In general, however, doses employed for adult human treatment are typically in the range of 0.01 mg to 5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED50 with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal. In some embodiments, an RBP4 inhibitor, a pharmaceutically acceptable salt, solvate, polymorph, prodrug, metabolite, N-oxide, stereoisomer, or isomer thereof is administered orally or parenterally to the subject in need thereof. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration. In some embodiments, an RBP4 inhibitor, a pharmaceutically acceptable salt, solvate, polymorph, prodrug, metabolite, N-oxide, stereoisomer, or isomer thereof is administered orally or intravenously to a subject in need thereof. In some embodiments, an RBP4 inhibitor, a pharmaceutically acceptable salt, solvate, polymorph, prodrug, metabolite, N-oxide, stereoisomer, or isomer thereof is administered orally to a subject in need thereof. In some embodiments, an RBP4 inhibitor, a pharmaceutically acceptable salt, solvate, polymorph, prodrug, metabolite, N-oxide, stereoisomer, or isomer thereof is administered intravenously to a subject in need thereof.

In any of the aforementioned aspects are further embodiments comprising single administrations of the effective amount of the compound, including further embodiments in which (i) the compound is administered once a day; or (ii) the compound is administered to the mammal multiple times over the span of one day, e.g., two, three, four or more times daily. In some embodiments, the RBP4 inhibitory compounds described herein are administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the RBP4 inhibitory compounds described herein, or a pharmaceutically acceptable salt, solvate, polymorph, prodrug, metabolite, N-oxide, stereoisomer, or isomer thereof, are administered daily.

In any of the aforementioned aspects are further embodiments comprising multiple administrations of the effective amount of the compound, including further embodiments in which (i) the compound is administered continuously or intermittently: as in a single dose; (ii) the time between multiple administrations is every 3 hours; (iii) the time between multiple administrations is every 6 hours; (iv) the compound is administered to the mammal every 8 hours; (v) the compound is administered to the mammal every 12 hours; or (vi) the compound is administered to the mammal every 24 hours.

In certain embodiments wherein a patient's status does improve, the dose of drug being administered is temporarily reduced or temporarily suspended for a certain length of time (e.g., a “drug holiday”). In specific embodiments, the length of the drug holiday is 2 days to 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 7 days. In one embodiment, the length of the drug holiday is 7 days. In one embodiment, the length of the drug holiday is 14 days. In one embodiment, the length of the drug holiday is 28 days.

In some instances, methods described herein comprise diagnosing, treating, or predicting therapeutic response from one or more composite biomarkers. In some instances, composite biomarkers comprise one or more variables. In some instances, one or more biomarkers is associated or predicted with a treatment or disease outcome. In some instances, composite biomarkers comprise 1, 2, 3, 4, 5, 6, 7, 8, 9, 10, or more than 10 biomarkers. In some embodiments the method comprises: (a) administering to the subject a treatment; (b) determining whether the treatment is therapeutically effective to treat the subject based on a composite biomarker, wherein the composite biomarker is calculated based on at least one variable, wherein at least one of the at least one variable relates to one or more selected from the group comprising vision, visual function, ocular anatomy, ocular physiology, ocular pathology, and a change thereof. In some embodiments the method comprises: (a) administering to the subject a treatment; (b) performing an assay on the subject to obtain at least one variable, wherein at least one of the at least one variable relates to one or more selected from the group comprising vision, visual function, ocular anatomy, ocular physiology, ocular pathology, and a change thereof; (c) calculating the at least one variable to yield a composite biomarker; and (d) determining whether the treatment is therapeutically effective to treat the subject based on the composite biomarker. In some embodiments the method comprises: (a) administering to the subject a treatment; (b) performing a first assay on the subject to obtain a first set of at least one variable at a first time point, wherein at least one of the at least one variable relates to one or more selected from the group comprising vision, visual function, ocular anatomy, ocular physiology, ocular pathology, and a change thereof; (c) calculating the first set of the at least one variable to yield a first composite biomarker; (d) performing a second assay on the subject to obtain a second set of the at least one variable at a second time point; (e) calculating the second set of the at least one variable to yield a second composite biomarker; and (f) determining whether the treatment is therapeutically effective to treat the subject based on the first and second composite biomarkers. In some embodiments the method comprises: (a) administering to the subject a treatment; (b) performing an assay on the subject to obtain at least one variable, wherein at least one of the at least one variable relates to one or more selected from the group comprising vision, visual function, ocular anatomy, ocular physiology, ocular pathology, and a change thereof, and (c) calculating the at least one variable to yield a composite biomarker, wherein the treatment is therapeutically effective to treat the subject when the composite biomarker exceeds a threshold value, wherein the treatment is not therapeutically effective to treat the subject when the composite biomarker does not exceed the threshold value. In some embodiments the method comprises: (a) administering to the subject a first dosage of a treatment; (b) performing an assay on the subject to obtain at least one variable, wherein at least one of the at least one variable relates to one or more selected from the group comprising vision, visual function, ocular anatomy, ocular physiology, ocular pathology, and a change thereof, (c) calculating the at least one variable to yield a composite biomarker; (d) determining whether the first dosage of the treatment is therapeutically effective to treat the subject based on the composite biomarker; and (e) administering to the subject a second dosage of the treatment, wherein the second dosage is different than the first dosage when the first dosage is determined to be not therapeutically effective to treat the subject, wherein the second dosage is about the same as the first dosage when the first dosage is determined to be therapeutically effective to treat the subject. In some embodiments the method comprises: (a) performing a first assay on the subject to obtain a first set of at least one variable, wherein at least one of the at least one variable relates to one or more selected from the group comprising vision, visual function, ocular anatomy, ocular physiology, ocular pathology, and a change thereof, (b) calculating the first set of the at least one variable to yield a first composite biomarker; (c) administering a treatment to the subject; (d) performing a second assay on the subject to obtain a second set of the at least one variable; (e) calculating the second set of the at least one variable to yield a second composite biomarker; and (f) determining whether the treatment is therapeutically effective to treat the subject based on the first and second composite biomarkers. In some embodiments the method comprises: (a) performing an assay on the subject to obtain at least one variable, wherein at least one of the at least one variable relates to one or more selected from the group comprising vision, visual function, ocular anatomy, ocular physiology, ocular pathology, and a change thereof; (b) calculating the at least one variable to yield a composite biomarker; (c) selecting the subject for a treatment based on the composite biomarker; and (d) administering the treatment to the subject. In some embodiments the method comprises: (a) performing a first assay on the subject to obtain a first set of at least one variable, wherein at least one of the at least one variable relates to one or more selected from the group comprising vision, visual function, ocular anatomy, ocular physiology, ocular pathology, and a change thereof, (b) calculating the first set of the at least one variable to yield a first composite biomarker; (c) selecting the subject for a treatment based on the first composite biomarker; (d) administering the treatment to the subject; (e) performing a second assay on the subject to obtain a second set of the at least one variable; (f) calculating the second set of the at least one variable to yield a second composite biomarker; and (g) determining whether the treatment is therapeutically effective to treat the subject based on the first and second composite biomarkers. In some embodiments the method comprises: (a) performing an assay on the subject to obtain at least one variable, wherein at least one of the at least one variable relates to one or more selected from the group comprising vision, visual function, ocular anatomy, ocular physiology, ocular pathology, and a change thereof, (b) calculating the at least one variable to yield a composite biomarker; (c) selecting the subject for the treatment when the composite biomarker exceeds a threshold value, or optionally, not selecting the subject for the treatment when the composite biomarker does not exceed the threshold value; and (d) administering to the subject the treatment when the subject is selected for the treatment, or optionally, not administering to the subject a treatment when the subject is not selected for the treatment. In some embodiments the method comprises: (a) performing a first assay on the subject to obtain a first set of at least one variable, wherein at least one of the at least one variable relates to one or more selected from the group comprising vision, visual function, ocular anatomy, ocular physiology, ocular pathology, and a change thereof; (b) calculating the first set of the at least one variable to yield a first composite biomarker; (c) selecting the subject for a treatment based on the first composite biomarker; (d) administering to the subject a first dosage of the treatment; (e) performing a second assay on the subject to obtain a second set of the at least one variable; (f) calculating the second set of the at least one variable to yield a second composite biomarker; (g) determining whether the first dosage of the treatment is therapeutically effective to treat the subject based on the first and second composite biomarkers; and (h) administering to the subject a second dosage of the treatment, wherein the second dosage is different than the first dosage when the first dosage is determined to be not therapeutically effective to treat the subject, wherein the second dosage is about the same as the first dosage when the first dosage is determined to be therapeutically effective to treat the subject. In some embodiments the method comprises: (a) performing a first assay on the subject to obtain a first set of at least one variable, wherein at least one of the at least one variable relates to one or more selected from the group comprising vision, visual function, ocular anatomy, ocular physiology, ocular pathology, and a change thereof, (b) calculating the first set of the at least one variable to yield a first composite biomarker; (d) administering to the subject a first dosage of a treatment; (e) performing a second assay on the subject to obtain a second set of the at least one variable; (f) calculating the second set of the at least one variable to yield a second composite biomarker; (g) determining that the first dosage of the treatment is therapeutically effective to treat the subject when the difference between the first and second composite biomarkers exceeds a threshold value, or optionally, determining that the first dosage of the treatment is not therapeutically effective to treat the subject when the difference between the first and second composite biomarkers does not exceed the threshold value; and (h) administering to the subject a second dosage of the treatment, wherein the second dosage is different than the first dosage when the first dosage is determined to be not therapeutically effective to treat the subject, wherein the second dosage is about the same as the first dosage when the first dosage is determined to be therapeutically effective to treat the subject. In some embodiments, the method comprises determining that a dosage of the treatment administered to the subject is therapeutically effective to treat the subject by calculating at least one variable of the subject to yield a composite biomarker, wherein at least one of the at least one variable relates to one or more selected from the group comprising vision, visual function, ocular anatomy, ocular physiology, ocular pathology, and a change thereof. In some embodiments, the method comprises administering to the subject a therapeutically effective dosage of a treatment, wherein the subject is selected for the treatment based on a composite biomarker, wherein the composite biomarker is calculated based on at least one variable of the subject, wherein at least one of the at least one variable relates to one or more selected from the group comprising vision, visual function, ocular anatomy, ocular physiology, ocular pathology, and a change thereof. In some embodiments, the method comprises selecting a subject with an ocular disease for a treatment based on a composite biomarker, wherein the composite biomarker is calculated based on at least one variable of the subject, wherein at least one of the at least one variable relates to one or more selected from the group comprising vision, visual function, ocular anatomy, ocular structure, ocular physiology, ocular pathology, and a change thereof. In some embodiments, the method comprises (a) calculating at least one variable of the subject to yield a composite biomarker, wherein at least one of the at least one variable relates to one or more selected from the group comprising vision, visual function, ocular anatomy, ocular physiology, ocular pathology, and a change thereof, and (b) selecting the subject for the treatment when the composite biomarker exceeds a threshold value, or optionally, not selecting the subject for the treatment when the composite biomarker does not exceed the threshold value. In some embodiments, the method comprises (a) performing an assay on the subject to obtain at least one variable, wherein at least one of the at least one variable relates to one or more selected from the group comprising vision, visual function, ocular anatomy, ocular physiology, ocular pathology, and a change thereof; (b) calculating the at least one variable to yield a composite biomarker; and (c) selecting the subject for the treatment when the composite biomarker exceeds a threshold value, or optionally, not selecting the subject for the treatment when the composite biomarker does not exceed the threshold value. In some embodiments, the method comprises (a) calculating at least one variable of the subject to yield a composite biomarker, wherein at least one of the at least one variable relates to one or more selected from the group comprising vision, visual function, ocular anatomy, ocular physiology, ocular pathology, and a change thereof, and (b) predicting the subject's therapeutic response to the treatment based on the composite biomarker. In some embodiments, the method comprises (a) performing an assay on the subject to obtain at least one variable, wherein at least one of the at least one variable relates to one or more selected from the group comprising vision, visual function, ocular anatomy, ocular physiology, ocular pathology, and a change thereof, (b) calculating the at least one variable to yield a composite biomarker; and (c) determining that the subject is likely to have therapeutic response to the treatment when the composite biomarker exceeds a threshold value, or optionally, determining that the subject is likely not to have therapeutic response to the treatment when the composite biomarker does not exceed the threshold value. In some embodiments, the method comprises determining a dosage of a treatment for a subject with an ocular disease based on a composite biomarker, wherein the composite biomarker is calculated based on at least one variable of the subject, wherein at least one of the at least one variable relates to one or more selected from the group comprising vision, visual function, ocular anatomy, ocular physiology, ocular pathology, and a change thereof. In some embodiments, the method comprises: (a) performing an assay on the subject to obtain at least one variable, wherein at least one of the at least one variable relates to one or more selected from the group comprising vision, visual function, ocular anatomy, ocular physiology, ocular pathology, and a change thereof; (b) calculating the at least one variable to yield the composite biomarker; and (c) determining a dosage of the treatment for the subject based on the composite biomarker. In some embodiments, the method comprises: (a) performing a first assay on the subject to obtain a first set of at least one variable, wherein at least one of the at least one variable relates to one or more selected from the group comprising vision, visual function, ocular anatomy, ocular physiology, ocular pathology, and a change thereof, (b) calculating the first set of the at least one variable to yield a first composite biomarker; (b) administering the treatment to the subject; (b) performing a second assay on the subject to obtain a second set of the at least one variable; (c) calculating the second set of the at least one variable to yield a second composite biomarker; and (d) determining a dosage of the treatment for the subject based on the first and second composite biomarkers. In some embodiments, the method comprises administering to a subject with an ocular disease a second dosage of a treatment, wherein a first dosage of the treatment previously administered to the subject was determined to lack therapeutic efficacy to treat the ocular disease in the subject based on a composite biomarker calculated from at least one variable of the subject, wherein at least one of the at least one variable relates to one or more selected from the group comprising vision, vision, visual function, ocular anatomy, ocular physiology, ocular pathology, and a change thereof, wherein the second dosage is different than the first dosage. In some embodiments, the method comprises determining a dosage of a treatment for an ocular disease in a subject, the method comprises: (a) administering to the subject a dosage of the treatment; (b) performing an assay on the subject to obtain at least one variable, wherein at least one of the at least one variable relates to one or more selected from the group comprising vision, visual function, ocular anatomy, ocular physiology, ocular pathology, and a change thereof, (c) calculating the at least one variable to yield a composite biomarker; (d) determining whether the dosage of the treatment is therapeutically effective to treat the subject with the ocular disease based on the composite biomarker. In some embodiments, the method comprises determining a dosage of a treatment for a subject with an ocular disease, the method comprises: (a) performing a first assay on the subject to obtain a first set of at least one variable, wherein at least one of the at least one variable relates to one or more selected from the group comprising vision, visual function, ocular anatomy, ocular physiology, ocular pathology, and a change thereof, (b) calculating the first set of the at least one variable to yield a first composite biomarker; (c) administering to the subject a dosage of the treatment; (d) performing a second assay on the subject to obtain a second set of the at least one variable; (e) calculating the second set of the at least one variable to yield a second composite biomarker; and (f) determining that the dosage of the treatment is therapeutically effective to treat the subject when the difference between the first and second composite biomarkers exceeds a threshold value, or optionally, determining that the dosage of the treatment is not therapeutically effective to treat the subject when the difference between the first and second composite biomarkers does not exceed the threshold value.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₅ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₁₅ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, ortrifluoromethyl), aralkyl (optionally substituted with halogen, hydroxy, methoxy, or trifluoromethyl), heterocyclyl (optionally substituted with halogen, hydroxy, methoxy, ortrifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl). [00265]“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, ortrifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C₅-C₈ alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where tis 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, ortrifluoromethyl), heterocyclyl (optionally substituted with halogen, hydroxy, methoxy, ortrifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bon d and to the radical group through a single bond. In certain embodiments, an alkynylene comprises two to eight carbon atoms (e.g., C₂-C₅ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atom (e.g., C₂ alkylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, ortrifluoromethyl), aralkyl (optionally substituted with halogen, hydroxy, methoxy, or trifluoromethyl), heterocyclyl (optionally substituted with halogen, hydroxy, methoxy, ortrifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, ortrifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, ortrifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula-R^{c}-carbocyclyl where R^{c} is an alkylene chain as defined above. The alkylene chain and the carbocyclyl radical are optionally substituted as defined above.

“Carbocyclylalkynyl” refers to a radical of the formula-R^{c}-carbocyclyl where R^{c} is an alkynylene chain as defined above. The alkynylene chain and the carbocyclyl radical are optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical are optionally substituted as defined above.

As used herein, “carboxylic acid bioisostere” refers to a functional group or moiety that exhibits similar physical, biological and/or chemical properties as a carboxylic acid moiety.

Examples of carboxylic acid bioisosteres include, but are not limited to,

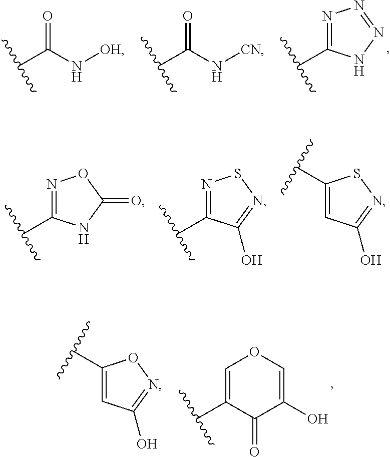

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, ortrifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, ortrifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)— or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

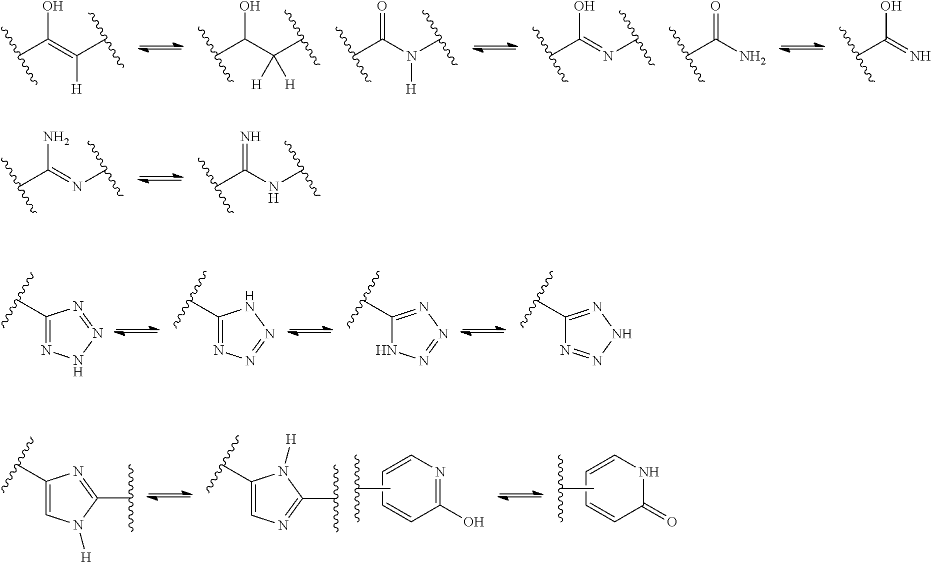

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

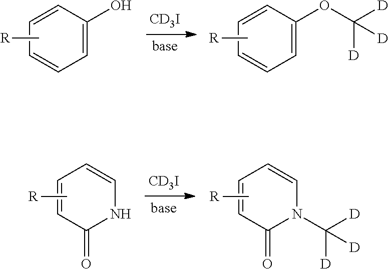

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

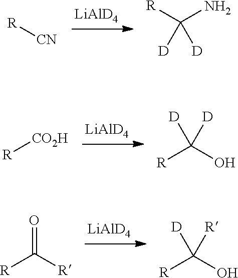

Deuterium gas and palladium catalyst are employed to reduce unsaturated carbon-carbon linkages and to perform a reductive substitution of aryl carbon-halogen bonds as illustrated, by way of example only, in the reaction schemes below.

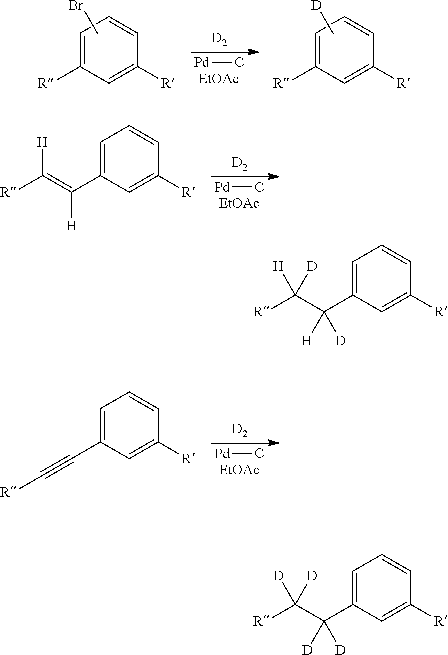

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the heterocyclic RBP4 inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

“Prodrug” is meant to indicate a compound that is, in some embodiments, converted under physiological conditions or by solvolysis to a biologically active compound described herein. Thus, the term “prodrug” refers to a precursor of a biologically active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compound s and the like.

### EXAMPLES

### Example 1: Clinical Treatment of STGD 1 and Therapeutic Evaluation

This example illustrates the use of a combination of variables for clinical evaluation of a treatment for STGD.

The therapeutic effect of a compound of Formula (I), also known as Tinlarebant, was evaluated in a group of individuals with adolescent STGD. The treatment covered Phase 1b and Phase 2. Phase 1b included a 4-week treatment and a 14-day follow-up. Phase 2 included a 2-year treatment and 1-month follow-up. A total of 13 individuals received the treatment: 5 males and 8 females. All 13 individuals had STGD 1 aged 12-18 years old. All 13 individuals received the treatment of a compound of Formula (I), a RBP4 inhibitor. The demographic information of the 13 individuals is listed in Table 1 and Table 2.

TABLE 1
Demographics and Clinical Information

| | | | | | VA at | BCVA at | | |
|---|---|---|---|---|---|---|---|---|
| | | | Age | | Onset | Enrollment | Lesion | |
| | | | at | Age at | (Right/ | (Right/ | Type/ | |
| Subject | Race | Gender | Onset | Enrollment | Left) | Left) | Location | Genotype |
| 1 | Caucasian | Female | 13 | 15 | 36/37 | 34/36 | Unifocal/ | c.1922G > C; c.2905A > G |
| | | | | | | | Fovea | |
| 2 | Caucasian | Female | 11 | 12 | 33/39 | 40/42 | Unifocal/ | c.768G > T (p.Val256=); |
| | | | | | | | Fovea | c.6006-1G > C; |
| | | | | | | | | c.1532G > A |
| | | | | | | | | (p.Arg5 11His) |
| 3 | Asian/ | Female | 13 | 13 | 49/44 | 49/48 | Unifocal/ | c.2894A > G; |
| | Caucasian | | | | | | Fovea | 4539 + 2028C > T |
| 4 | Caucasian | Female | 14.4 | 15 | 39/44 | 45/45 | Unifocal/ | c.2576del; c.2588G > C; |
| | | | | | | | Fovea | c.5603A > T |
| 5 | Caucasian | Female | 12.2 | 13 | 70/61 | 45/55 | Unifocal/ | c.3260A > G;c.5714+5G > |
| | | | | | | | Fovea | A |
| 6 | Caucasian | Female | 13.8 | 18 | 70/65 | 35/36 | Unifocal/ | c.5461- |
| | | | | | | | Fovea | 10T > C;c.6079C > T |
| | | | | | | | | (p.L2027F) |
| 7 | Caucasian | Male | 15.3 | 15 | 59/75 | 69/40 | Unifocal/ | c.634C > T;c.2894A > G |
| | | | | | | | Fovea | |
| 8 | Caucasian | Male | 8.1 | 13 | 57/57 | 31/31 | Unifocal/ | c.161G > A |
| | | | | | | | Fovea | p.(Cys54Tyr);c.4139C > T |
| | | | | | | | | p.(Pro1380Leu) |
| 9 | Asian | Male | 10.5 | 13 | 50/59 | 45/35 | Unifocal/ | c.[5645T > C];[1804C > T] |
| | | | | | | | Fovea | |
| 10 | Asian | Male | 9.5 | 12 | 50/35 | 45/35 | Unifocal/ | c.[5645T > C];[1804C > T] |
| | | | | | | | Fovea | |
| 11 | Asian | Female | 13 | 14 | 59/59 | 50/50 | Unifocal/ | c.1804C > T(;)5761G > A |
| | | | | | | | Fovea | |
| 12 | Asian | Female | 11 | 18 | 35/44 | 35/35 | Unifocal/ | c.[1804C > T];[5645T > C] |
| | | | | | | | Fovea | |
| 13 | Asian | Male | 8 | 12 | 59/59 | 35/35 | Unifocal/ | c.[1804C > T];[5645T > C] |
| | | | | | | | Fovea | |

TABLE 2
Gender and Race Composition of Patients

| | Male | Female | |
|---|---|---|---|
| Gender | 62% | 38% | |
| | Caucasian | Asian | Others |
| Race | 54% | 38% | 8% |

The clinical study evaluated ocular and systemic safety and tolerability of the compound and determined the optimal dose, pharmacokinetics (PK), and pharmacokinetics (PD). The study measured visual improvement by best corrected visual acuity (BCVA) test, self-reported visual questionnaire test, the change in lesion size by FAF, the change in retinal thickness and morphology by SD-OCT, the change in retinal sensitivity by microperimetry, and the change in quantitative AF. In addition, this study evaluated correlation between reduction in RBP4 and rate of lesion growth including change in autofluorescence.

Overall, 5 mg daily of the compound was shown to be safe and well tolerated in adolescent STGD1 patients. In addition, the treatment was able to maintain 80%-90% RBP4 reduction from baseline throughout the study. All patients received at least 7 months of the treatment. Adverse events were observed in patients, as described in Table 3. Delayed dark adaptation, night vision impairment, and xanthopsia/chromatopsia were the only drug related ophthalmic adverse events. All instances of delayed dark adaptation, night vision impairment, and xanthopsia/chromatopsia were mild and transient. Patients determined to have delayed dark adaptation were mostly asymptomatic. No severe adverse events or severe adverse events were found and no adverse events required discontinuation of treatment. No clinical significant findings were observed in relation to vital signs, physical exams, electrocardiogram (ECG), and organ functions.

TABLE 3
Related Adverse Events

| | Frequency | | |
|---|---|---|---|
| Related Adverse Events | (subject) | % Recovered | % On-going |
| Xanthopsia/Chromatopsia | 9/13 | 6/9 (66.67%) | 3/9 (33.33%) |
| Delayed Dark Adaptation | 8/13 | 2/8 (25%) | 6/8 (75%) |
| Night Vision Impairment | 1/13 | 0/1 | 1/1 (100%) |

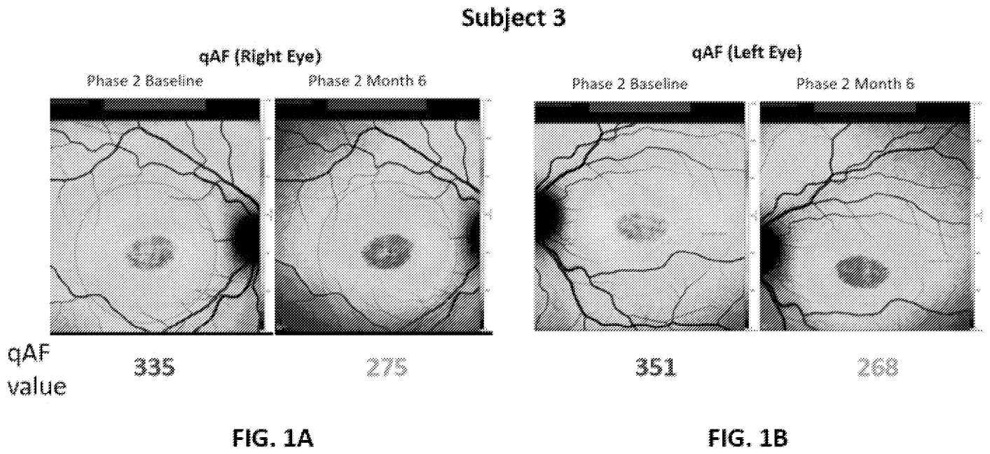

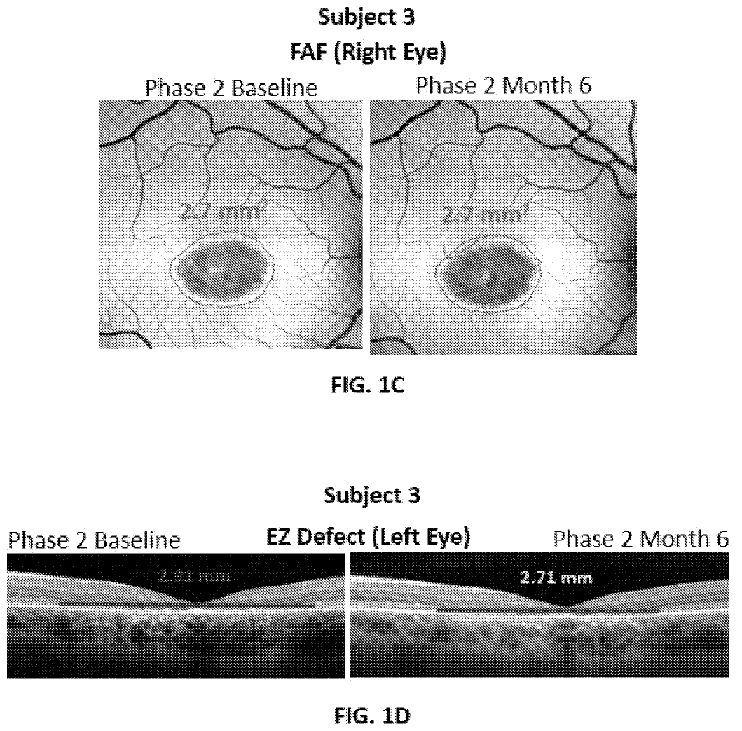

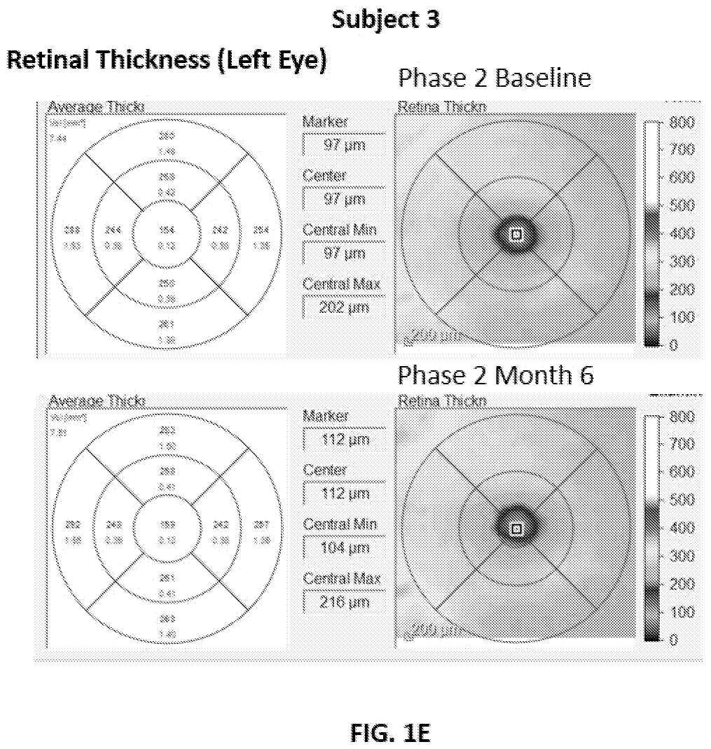

The clinical evaluation of some of the patients was summarized as follows. Subject 3 is a 13-year female patient and was diagnosed with STGD 1 at the age of 13. During phase 2 clinical trial, subject 3 has no increase in the area size of questionable decrease of autofluorescence (QDAF) in either left or right eye compared between week 1 and week 9. During phase 2 clinical trial, the ellipsoid zone (EZ) width of subject 3 decreased in both eyes, especially in the left eye, compared between week 1 and week 9. Overall, there was little to no change in central subfield retina thickness (CST), middle subfield retina thickness (MST), or outer subfield retina thickness (OST). During phase 2 clinical trial between week 1 and week 9, there was a decrease in autofluorescence (AF) based on quantitative AF in both right eye (FIG. 1A) and left eye (FIG. 1B), especially in the left eye (FIG. 1B). FAF, retinal thickness, and EZ Defect scores were similar for both visits (FIGS. 1C-1E).

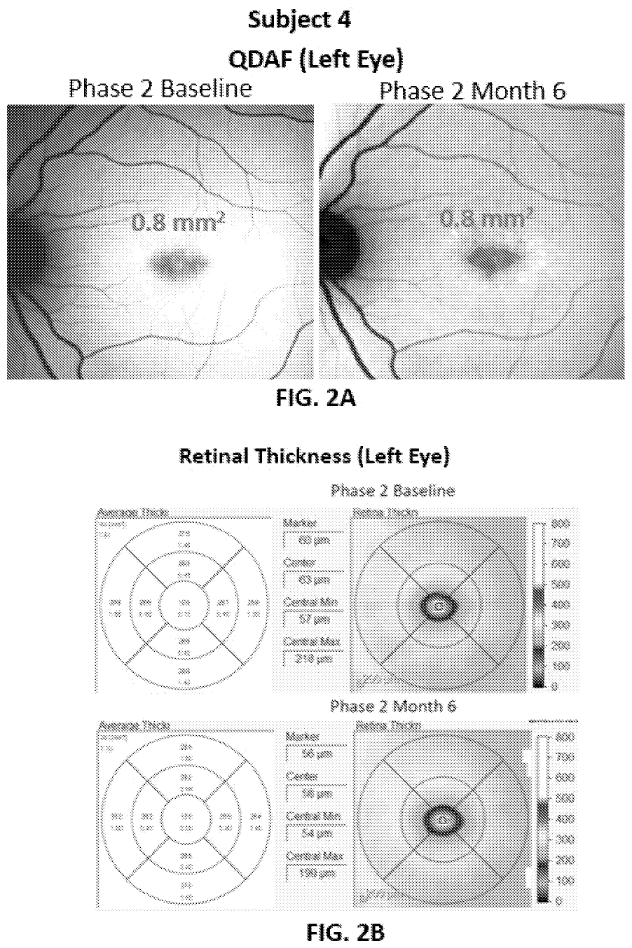

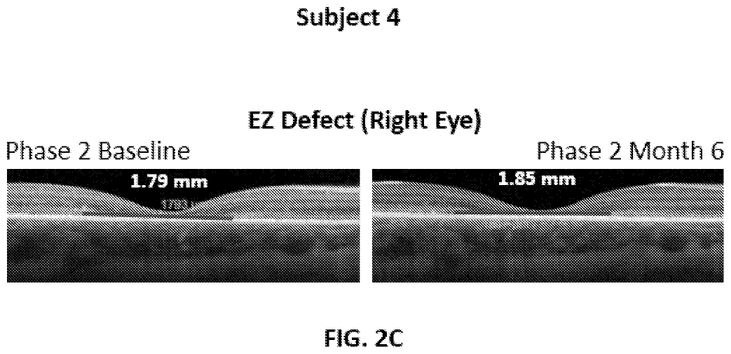

Subject 4 is a 15-year female patient and was diagnosed with STGD 1 at the age of 14.4. In BCVA test, subject 4 gained 3 ETDRS letters in both eyes. During phase 2 clinical trial between week 1 and week 9, there was no increase of QDAF in size in the left eye (FIG. 2A) or right eye. During phase 2 clinical trial between week 1 and week 9, there was little to no change of MST in either eye and an increase of OST in both eyes, especially left eye (FIG. 2B). EZ defect changes are shown in FIG. 2C.

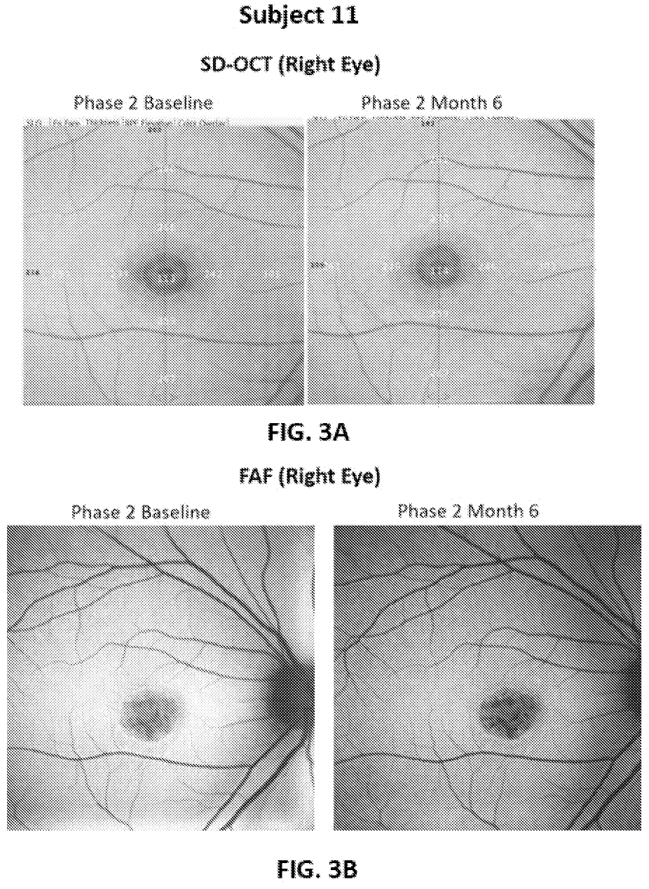

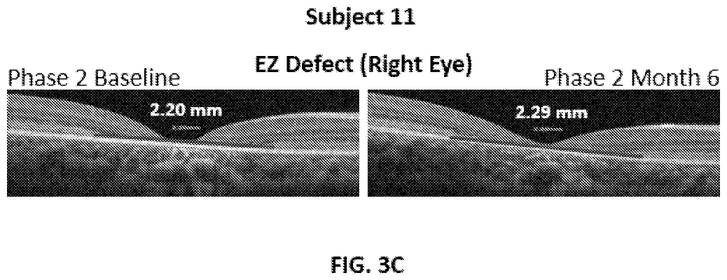

Subject 11 is a 14-year female patient and was diagnosed with STGD 1 at the age of 13. In BCVA test, subject 11 gained 6 letters in the left eye and there was no change in the right eye. During phase 2 clinical trial between week 1 and week 9, the EZ width stabilized in both right eye (FIG. 3A) and left eye. During phase 2 clinical trial between week 1 and week 9, the was little to no change of CST or MST in either left eye or right eye (FIG. 3A), while there was an increase of OST in both left eye and right eye (FIG. 3A). During phase 2 clinical trial between week 1 and week 9, there was a transition of QDAF to DDAF in both right eye (FIG. 3B) and left eye.

Under the BCVA test, 8 out of the 13 patients showed visual improvement in at least one eye, including 2 patients with visual improvement in both eyes. The overall BCVA test exhibited an average of −2.8±1.85 letters of loss in the right eye, and 1.9±1.32 letters of gain in the left eye. One patient, subject 10, gained 9 letters in both eyes after approximately 7-month treatment. Prior to the treatment at the age of 12, subject 10's vision had deteriorated to 35 letters in both eyes since being diagnosed with STGD 1 at the age of 9. Subject 4 experienced a consistent improvement in both eyes over the 7-month treatment period, from 45 to 50, then to 53 letters in both eyes.

Under the FAF test, there was a trend of preventing or slowing autofluorescence expansion which indicated slowing of cell death. 12 of the 13 patients maintained no DDAF lesion growth. 6 of 11 patients had no change or reduction in QDAF area in one or both eyes. The average change in QDAF area was 0.3±0.11 mm² in the right eye and 0.1±0.11 mm² in the left eye.

In regard to the total retinal thickness by distance to fovea, 5 patients had no to little change in CST in at least one eye and the study cohort had an average thinning of 10.27±2.2 microns in the right eye, and 7.6±2.14 microns in the left eye. 5 patients had no change or growth in MST in at least one eye and the study cohort had an average thinning of 6.27±2.03 microns in the right eye, and 7.2±2.54 microns in the left eye. 4 patients had improvement (i.e., increase in thickness) or no change in OST in both eyes and the study cohort had an average thinning of 3.8±3.08 microns in the right eye, and 3.11±3.5 microns in the left eye. Subject 11 had a growth of 15 and 16 microns in both eyes. Subject 4 had a growth of 2 and 8 microns in both eyes.

The patients exhibited the following changes in the EZ. 5 patients showed improvement in EZ defect width (i.e., narrowing) in at least one eye, including 3 patients showing improvements in both eyes. Subject 3 had a 0.13 mm reduction in the right eye, and a 0.20 mm reduction in the left eye. Subject 6 had a 0.27 mm reduction in the right eye, and 0.22 mm reduction in the left eye. The study cohort had an average increase of 0.39±0.27 mm in the right eye, and 0.48±0.26 mm in the left eye. Changes in retinal thickness are shown in Table 4.

TABLE 4
Retinal Thickness-Cohort Means

| | Right Eye | Left Eye |
|---|---|---|
| Region of Interest | (mean ± SD, μm) | (mean ± SD, μm) |
| CST | −10.15 ± 1.5 | −8.83 ± 1.91 |
| MST | −6.77 ± 1.74 | −7.92 ± 2.13 |
| OST | −8.08 ± 2.47 | −7.60 ± 2.62 |

The 6-month data in the right eye were used to analyze the relationship between the different assessments described above Tables 5-6.

TABLE 5
Spearman Rank Correlation Coefficient

| | | | | | | | EZ | | | | | |
|---|---|---|---|---|---|---|---|---|---|---|---|---|
| | VA | VQ | DDAF | QDAF | DAF | RPE | WIDTH | CST | MST | OST | MP | QAF |
| VA | | | | | | | | | | | | |
| VQ | 0.104 | | | | | | | | | | | |
| DDAF | 0.000 | −0.407 | | | | | | | | | | |
| QDAF | −0.235 | 0.433 | −0.550 | | | | | | | | | |
| DAF | −0.277 | 0.197 | −0.138 | 0.899 | | | | | | | | |
| RPE | −0.407 | −0.524 | −0.111 | | | | | | | | | |
| EZ | −0.849 | −0.018 | −0.332 | 0.901 | 0.955 | 0.581 | | | | | | |
| WIDTH | | | | | | | | | | | | |
| CST | 0.210 | 0.000 | 0.524 | 0.134 | 0.487 | −0.407 | −0.060 | | | | | |
| MST | 0.006 | −0.067 | 0.522 | 0.025 | 0.326 | −0.522 | −0.168 | 0.608 | | | | |
| OST | 0.548 | 0.647 | | −0.143 | −0.143 | −0.577 | −0.393 | 0.371 | 0.333 | | | |
| MP | 0.322 | −0.286 | 0.290 | −0.226 | −0.126 | −0.522 | −0.707 | 0.134 | 0.479 | 0.095 | | |
| QAF | −1.000 | −1.000 | | 1.000 | 1.000 | | 1.000 | 1.000 | 1.000 | 1.000 | 1.000 | −1.000 |

TABLE 6
P-value

| | VA | VQ | DDAF | QDAF | DAF | RPE | EZWIDTH | CST | MST | OST | MP | QAF |
|---|---|---|---|---|---|---|---|---|---|---|---|---|
| VA | | | | | | | | | | | | |
| VQ | 0.78 | | | | | | | | | | | |
| DDAF | 1.00 | 0.24 | | | | | | | | | | |
| QDAF | 0.54 | 0.24 | 0.12 | | | | | | | | | |
| DAF | 0.47 | 0.61 | 0.72 | 0.00 | | | | | | | | |
| RPE | 0.24 | 0.12 | 0.76 | | | | | | | | | |
| EZWIDTH | 0.01 | 0.97 | 0.42 | 0.01 | 0.00 | 0.13 | | | | | | |
| CST | 0.56 | 1.00 | 0.12 | 0.73 | 0.18 | 0.24 | 0.89 | | | | | |
| MST | 0.99 | 0.85 | 0.12 | 0.95 | 0.39 | 0.12 | 0.69 | 0.06 | | | | |
| OST | 0.16 | 0.08 | | 0.76 | 0.76 | 0.13 | 0.38 | 0.37 | 0.42 | | | |
| MP | 0.36 | 0.42 | 0.42 | 0.56 | 0.75 | 0.12 | 0.05 | 0.71 | 0.16 | 0.82 | | |
| QAF | | | | | | | | | | | | |

The results showed that the EZ defect width was highly correlated with visual acuity (VA), QDAF, and DAF with a correlation coefficient higher than 0.8 for each of them in the right eye and a p value less than 0.05. These results correlate visual acuity with structural (EZ defect) and pathological (QDAF/DAF as a measure of AF) change. More of the features had trends (correlation coefficient>0.5) but without scientific significance. Multivariate analysis confirmed significant correlation of EZ Defect Width and DAF with VA (p-value<0.05).

RBP4 is the sole transporter of retinol (vitamin A) to the retina. Retinol is the primary material for bisretinoids/A2E formation. Based on these results, the compound of Formula (I) reduced supply of retinol into the retina by suppressing RBP4, the sole retinol transporter for retina. The results showed a trend in preventing or slowing autofluorescence expansion, indicating slowing of cell death and a trend in reducing EZ defect width. Majority of the patients showed visual improvement in at least one eye. After approximately 7 months of treatment (1 month in Phase 1b and 6 months in Phase 2), the compound of Formula (I) was shown to be safe and well tolerated.

### Example 2: Prediction Model for Clinical Evaluation

This example illustrates a prediction model related to the response variable disclosed herein. The prediction model is constructed with a link function based on the magnitude of the variance of each feature variable as a function of its predicted value. The prediction model consists of three components. The first component is a predictor based on a function of regressors (ηᵢ). The second component is a model structure best fitting the conditional distribution of the response variable, Yᵢ, given the values of the explanatory variables in the model. The third component is a smooth and invertible link function g(⋅), which transforms the expectation of the response variable, μᵢ=E(Yᵢ), to the predictor: g(μᵢ)=ηᵢ=α+β₁ Xᵢ₁+β₂ Xᵢ₂+ . . . + βₖ Xᵢₖ. Based on the results in example 1, a prediction model can be constructed using visual acuity (VA) and EZ Width as follows: VA=−0.0896 EZ WIDTH+0.0701, Z-score=−2.211, P value=0.027, and CI 95% [−0.169, −0.010].

### Example 3: Clinical Evaluation

Using the general model of Example 2, one or more variables from Table 5 are measured from a patient either prior to or during treatment with an RBP4 inhibitor described herein (e.g., any structure of Table B, such as compound 1). In some embodiments variables include one or more of EZWIDTH, QDAF, and DAF. After treatment or continued treatment, variables are measured again and used to assess the patient's progress and/or response to the treatment. Based on this assessment, drug dosage, administration frequency, drug type, or other treatment variable is modified to improve the patient's treatment outcome.

### Example 4: Patient Selection

Using the general model of Example 2, one or more variables from Table 5 are measured from a potential patient. In some embodiments variables include one or more of EZWIDTH, QDAF, and DAF. These variables are used to assess the patient's potential response to treatment with an RBP4 inhibitor described herein (e.g., any structure of Table B, such as compound 1). Patients who are found to be more likely to respond favorably to the treatment are administered the inhibitor.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A method of treating a subject with an ocular disease, the method comprising:
(a) administering to the subject a treatment;
(b) determining whether the treatment is therapeutically effective to treat the subject based on a composite biomarker,
(c) predicting whether the prognostic potentials of treatment improvement based on a composite biomarker
wherein the composite biomarker is calculated based on at least two variables,
wherein at least one of the at least two variables relates to one or more selected from the group comprising vision, visual function, ocular anatomy, ocular physiology, ocular pathology, and a change thereof.

2. A method of treating a subject with an ocular disease, the method comprising:
(a) administering to the subject a treatment;
(b) performing an assay on the subject to obtain at least two variables, wherein at least one of the at least two variables relates to one or more selected from the group comprising vision, visual function, ocular anatomy, ocular physiology, ocular pathology, and a change thereof,
(c) calculating the at least two variables to yield a composite biomarker; and
(d) determining whether the treatment is therapeutically effective to treat the subject based on the composite biomarker.

3. A method of treating a subject with an ocular disease, the method comprising:
(a) administering to the subject a treatment;
(b) performing a first assay on the subject to obtain a first set of at least two variables at a first time point, wherein at least one of the at least two variables relates to one or more selected from the group comprising vision, visual function, ocular anatomy, ocular physiology, ocular pathology, and a change thereof,
(c) calculating the first set of the at least two variables to yield a first composite biomarker;
(d) performing a second assay on the subject to obtain a second set of the at least two variables at a second time point;
(e) calculating the second set of the at least two variables to yield a second composite biomarker; and
(f) determining whether the treatment is therapeutically effective to treat the subject based on the first and second composite biomarkers.

4. A method of treating a subject with an ocular disease, the method comprising:
(a) administering to the subject a treatment;
(b) performing an assay on the subject to obtain at least two variables, wherein at least one of the at least two variables relates to one or more selected from the group comprising vision, visual function, ocular anatomy, ocular physiology, ocular pathology, and a change thereof, and
(c) calculating the at least two variables to yield a composite biomarker,
wherein the treatment is therapeutically effective to treat the subject when the composite biomarker exceeds a threshold value, wherein the treatment is not therapeutically effective to treat the subject when the composite biomarker does not exceed the threshold value.

5. A method of treating a subject with an ocular disease, the method comprising:
(a) administering to the subject a first dosage of a treatment;
(b) performing an assay on the subject to obtain at least two variables, wherein at least one of the at least two variables relates to one or more selected from the group comprising vision, visual function, ocular anatomy, ocular physiology, ocular pathology, and a change thereof,
(c) calculating the at least two variables to yield a composite biomarker;
(d) determining whether the first dosage of the treatment is therapeutically effective to treat the subject based on the composite biomarker; and
(e) administering to the subject a second dosage of the treatment, wherein the second dosage is different than the first dosage when the first dosage is determined to be not therapeutically effective to treat the subject, wherein the second dosage is about the same as the first dosage when the first dosage is determined to be therapeutically effective to treat the subject.

6. A method of treating a subject with an ocular disease, the method comprising:
(a) performing a first assay on the subject to obtain a first set of at least two variables, wherein at least one of the at least two variables relates to one or more selected from the group comprising vision, visual function, ocular anatomy, ocular physiology, ocular pathology, and a change thereof,
(b) calculating the first set of the at least two variables to yield a first composite biomarker;
(c) administering a treatment to the subject;
(d) performing a second assay on the subject to obtain a second set of the at least two variables;
(e) calculating the second set of the at least two variables to yield a second composite biomarker; and
(f) determining whether the treatment is therapeutically effective to treat the subject based on the first and second composite biomarkers.

7. A method of treating a subject with an ocular disease, the method comprising:
(a) performing an assay on the subject to obtain at least two variables, wherein at least one of the at least two variables relates to one or more selected from the group comprising vision, visual function, ocular anatomy, ocular physiology, ocular pathology, and a change thereof,
(b) calculating the at least two variables to yield a composite biomarker;
(c) selecting the subject for a treatment based on the composite biomarker; and
(d) administering the treatment to the subject.

8. A method of treating a subject with an ocular disease, the method comprising:
(a) performing a first assay on the subject to obtain a first set of at least two variables, wherein at least one of the at least two variables relates to one or more selected from the group comprising vision, visual function, ocular anatomy, ocular physiology, ocular pathology, and a change thereof,
(b) calculating the first set of the at least two variables to yield a first composite biomarker;
(c) selecting the subject for a treatment based on the first composite biomarker;
(d) administering the treatment to the subject;
(e) performing a second assay on the subject to obtain a second set of the at least two variables;
(f) calculating the second set of the at least two variables to yield a second composite biomarker; and
(g) determining whether the treatment is therapeutically effective to treat the subject based on the first and second composite biomarkers.

9. A method of treating a subject with an ocular disease, the method comprising:
(a) performing an assay on the subject to obtain at least two variables, wherein at least one of the at least two variables relates to one or more selected from the group comprising vision, visual function, ocular anatomy, ocular physiology, ocular pathology, and a change thereof,
(b) calculating the at least two variables to yield a composite biomarker;
(c) selecting the subject for the treatment when the composite biomarker exceeds a threshold value, or optionally, not selecting the subject for the treatment when the composite biomarker does not exceed the threshold value; and
(d) administering to the subject the treatment when the subject is selected for the treatment, or optionally, not administering to the subject a treatment when the subject is not selected for the treatment.

10. A method of treating a subject with an ocular disease, the method comprising:
(a) performing a first assay on the subject to obtain a first set of at least two variables, wherein at least one of the at least two variables relates to one or more selected from the group comprising vision, visual function, ocular anatomy, ocular physiology, ocular pathology, and a change thereof,
(b) calculating the first set of the at least two variables to yield a first composite biomarker;
(c) selecting the subject for a treatment based on the first composite biomarker;
(d) administering to the subject a first dosage of the treatment;
(e) performing a second assay on the subject to obtain a second set of the at least two variables;
(f) calculating the second set of the at least two variables to yield a second composite biomarker;
(g) determining whether the first dosage of the treatment is therapeutically effective to treat the subject based on the first and second composite biomarkers; and
(h) administering to the subject a second dosage of the treatment, wherein the second dosage is different than the first dosage when the first dosage is determined to be not therapeutically effective to treat the subject, wherein the second dosage is about the same as the first dosage when the first dosage is determined to be therapeutically effective to treat the subject.

11. A method of treating a subject with an ocular disease, the method comprising:
(a) performing a first assay on the subject to obtain a first set of at least two variables, wherein at least one of the at least two variables relates to one or more selected from the group comprising vision, visual function, ocular anatomy, ocular physiology, ocular pathology, and a change thereof,
(b) calculating the first set of the at least two variables to yield a first composite biomarker;
(c) administering to the subject a first dosage of a treatment;
(d) performing a second assay on the subject to obtain a second set of the at least two variables;
(e) calculating the second set of the at least two variables to yield a second composite biomarker;
(f) determining that the first dosage of the treatment is therapeutically effective to treat the subject when the difference between the first and second composite biomarkers exceeds a threshold value, or optionally, determining that the first dosage of the treatment is not therapeutically effective to treat the subject when the difference between the first and second composite biomarkers does not exceed the threshold value; and
(g) administering to the subject a second dosage of the treatment, wherein the second dosage is different than the first dosage when the first dosage is determined to be not therapeutically effective to treat the subject, wherein the second dosage is about the same as the first dosage when the first dosage is determined to be therapeutically effective to treat the subject.

12. A method of monitoring the outcome of a treatment for an ocular disease in a subject, the method comprising determining that a dosage of the treatment administered to the subject is therapeutically effective to treat the subject by calculating at least two variables of the subject to yield a composite biomarker, wherein at least one of the at least two variables relates to one or more selected from the group comprising vision, visual function, ocular anatomy, ocular physiology, ocular pathology, and a change thereof.

13. A method of treating a subject with an ocular disease comprising administering to the subject a therapeutically effective dosage of a treatment, wherein the subject is selected for the treatment based on a composite biomarker, wherein the composite biomarker is calculated based on at least two variables of the subject, wherein at least one of the at least two variables relates to one or more selected from the group comprising vision, visual function, ocular anatomy, ocular physiology, ocular pathology, and a change thereof.

14. A method comprising selecting a subject with an ocular disease for a treatment based on a composite biomarker, wherein the composite biomarker is calculated based on at least two variables of the subject, wherein at least one of the at least two variables relates to one or more selected from the group comprising vision, visual function, ocular anatomy, ocular structure, ocular physiology, ocular pathology, and a change thereof.

15. A method of selecting a subject with an ocular disease for a treatment, the method comprising:
(a) calculating at least two variables of the subject to yield a composite biomarker, wherein at least one of the at least two variables relates to one or more selected from the group comprising vision, visual function, ocular anatomy, ocular physiology, ocular pathology, and a change thereof, and
(b) selecting the subject for the treatment when the composite biomarker exceeds a threshold value, or optionally, not selecting the subject for the treatment when the composite biomarker does not exceed the threshold value.

16. A method of selecting a subject with an ocular disease for a treatment, the method comprising:
(a) performing an assay on the subject to obtain at least two variables, wherein at least one of the at least two variables relates to one or more selected from the group comprising vision, visual function, ocular anatomy, ocular physiology, ocular pathology, and a change thereof,
(b) calculating the at least two variables to yield a composite biomarker; and
(c) selecting the subject for the treatment when the composite biomarker exceeds a threshold value, or optionally, not selecting the subject for the treatment when the composite biomarker does not exceed the threshold value.

17. A method of predicting a subject's therapeutic response to a treatment for an ocular disease, the method comprising:
(a) calculating at least two variables of the subject to yield a composite biomarker, wherein at least one of the at least two variables relates to one or more selected from the group comprising vision, visual function, ocular anatomy, ocular physiology, ocular pathology, and a change thereof, and
(b) predicting the subject's therapeutic response to the treatment based on the composite biomarker.

18. A method of predicting a subject's therapeutic response to a treatment for an ocular disease, the method comprising:
(a) performing an assay on the subject to obtain at least two variables, wherein at least one of the at least two variables relates to one or more selected from the group comprising vision, visual function, ocular anatomy, ocular physiology, ocular pathology, and a change thereof,
(b) calculating the at least two variables to yield a composite biomarker; and
(c) determining that the subject is likely to have therapeutic response to the treatment when the composite biomarker exceeds a threshold value, or optionally, determining that the subject is likely not to have therapeutic response to the treatment when the composite biomarker does not exceed the threshold value.

19. A method comprising determining a dosage of a treatment for a subject with an ocular disease based on a composite biomarker, wherein the composite biomarker is calculated based on at least two variables of the subject, wherein at least one of the at least two variables relates to one or more selected from the group comprising vision, visual function, ocular anatomy, ocular physiology, ocular pathology, and a change thereof.

20. A method of determining a dosage of a treatment for a subject with an ocular disease, the method comprising:
(a) performing an assay on the subject to obtain at least two variables, wherein at least one of the at least two variables relates to one or more selected from the group comprising vision, visual function, ocular anatomy, ocular physiology, ocular pathology, and a change thereof,
(b) calculating the at least two variables to yield the composite biomarker; and
(c) determining a dosage of the treatment for the subject based on the composite biomarker.

21. A method of determining a dosage of a treatment for an ocular disease in a subject, the method comprising:
(a) performing a first assay on the subject to obtain a first set of at least two variables, wherein at least one of the at least two variables relates to one or more selected from the group comprising vision, visual function, ocular anatomy, ocular physiology, ocular pathology, and a change thereof,
(b) calculating the first set of the at least two variables to yield a first composite biomarker;
(c) administering the treatment to the subject;
(d) performing a second assay on the subject to obtain a second set of the at least two variables;
(e) calculating the second set of the at least two variables to yield a second composite biomarker; and
(f) determining a dosage of the treatment for the subject based on the first and second composite biomarkers.

22. A method comprising administering to a subject with an ocular disease a second dosage of a treatment, wherein a first dosage of the treatment previously administered to the subject was determined to lack therapeutic efficacy to treat the ocular disease in the subject based on a composite biomarker calculated from at least two variables of the subject, wherein at least one of the at least two variables relates to one or more selected from the group comprising vision, vision, visual function, ocular anatomy, ocular physiology, ocular pathology, and a change thereof, wherein the second dosage is different than the first dosage.

23. A method of determining a dosage of a treatment for an ocular disease in a subject, the method comprising:
(a) administering to the subject a dosage of the treatment;
(b) performing an assay on the subject to obtain at least two variables, wherein at least one of the at least two variables relates to one or more selected from the group comprising vision, visual function, ocular anatomy, ocular physiology, ocular pathology, and a change thereof,
(c) calculating the at least two variables to yield a composite biomarker;
(d) determining whether the dosage of the treatment is therapeutically effective to treat the subject with the ocular disease based on the composite biomarker.

24. A method of determining a dosage of a treatment for a subject with an ocular disease, the method comprising:
(a) performing a first assay on the subject to obtain a first set of at least two variables, wherein at least one of the at least two variables relates to one or more selected from the group comprising vision, visual function, ocular anatomy, ocular physiology, ocular pathology, and a change thereof,
(b) calculating the first set of the at least two variables to yield a first compo site biomarker;
(c) administering to the subject a dosage of the treatment;
(d) performing a second assay on the subject to obtain a second set of the at least two variables;
(e) calculating the second set of the at least two variables to yield a second composite biomarker; and
(f) determining that the dosage of the treatment is therapeutically effective to treat the subject when the difference between the first and second composite biomarkers exceeds a threshold value, or optionally, determining that the dosage of the treatment is not therapeutically effective to treat the subject when the difference between the first and second composite biomarkers does not exceed the threshold value.

25. The method of any one of the preceding claims, wherein the ocular disease comprises maculopathy, retinopathy, retina atrophy, macular atrophy, macular degeneration, age-related macular degeneration (AMD), Stargardt's Disease (STGD), RP (Retinitis pigmentosa), an ABCA4 gene mutation, or a combination thereof.

26. The method of any one of the preceding claims, wherein a sample from the subject with the ocular disease has an expression level of retinol binding protein 4 (RBP4) of at least a threshold value.

27. The method of claim 26, wherein the threshold value is about 25 μg/ml.

28. The method of claim 26, wherein the threshold value is about 35 μg/ml.

29. The method of claim 26, wherein the threshold value is between about 25 μg/ml and about 100 μg/ml.

30. The method of any one of claims 26-29, wherein the expression level of RBP4 is measured by an assay comprising an antibody assay, an electrophoresis assay, an immunoassay, a radioimmunoassay, a chromatographic assay, a mass spectrometry assay, a microarray-based detection assay, a polymerase chain reaction assay, a sequencing assay, an immunohistochemistry assay, or any combination thereof.

31. The method of claim 30, wherein the antibody assay comprises ELISA.

32. The method of any one of claims 26-31, wherein a sample from the subject with the ocular disease has an expression level of vitamin A of at least a threshold value.

33. The method of claim 32, wherein the threshold value is about 150 ng/mL.

34. The method of claim 32, wherein the threshold value is about 225 ng/mL or about 390 ng/mL.

35. The method of claim 32, wherein the threshold value is between about 150 ng/mL and about 500 ng/mL.

36. The method of any one of claims 32-35, wherein the expression level of vitamin is measured by an assay comprising an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof.

37. The method of claim 26 or 32, wherein the sample comprises a blood sample.

38. The method of claim 37, wherein the expression level of RBP4 or vitamin A is measured from plasma or serum derived from the blood sample.

39. The method of any one of claims 1-38, wherein the presence or absence of one or more genomic variants indicates that the subject has the ocular disease.

40. The method of any one of claims 1-38, wherein the presence or absence of one or more genomic variants indicates that the subject has Stargardt disease.

41. The method of any one of claims 1-40, wherein the presence or absence of one or more genomic variants selected from the group comprising rs4147863, rs2275029, rs1800739, rs4147857, rs4147856, rs1801555, and rs1801574 indicates that the subject has the ocular disease.

42. The method of claim 39 or 40, wherein the one or more genomic variants comprises at least four of rs4147863, rs2275029, rs1800739, rs4147857, rs4147856, rs1801555, or rs1801574.

43. The method of claim 39 or 40, wherein the one or more genomic variants comprises at least five of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555, rs1762114, rs55860151, rs1800549, rs3112831, rs4147830, rs2297634, or rs4847281.

44. The method of any one of claims 1-43, wherein the treatment comprises a pharmaceutical composition comprising an RBP4 inhibitor or a compound that is configured to reduce blood RBP4 concentration in the subject.

45. The method of claim 44, wherein the pharmaceutical composition comprises a compound having the structure of Formula (I): 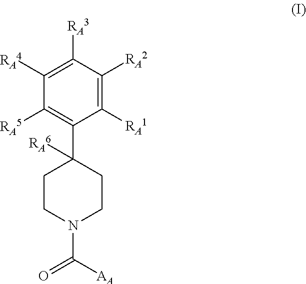
wherein:
R_{A}¹, R_{A}², R_{A}³, R_{A}⁴, and R_{A}⁵ are each independently H, halogen, CF₃, or C₁-C₄ alkyl, wherein two or more of R_{A}¹, R_{A}², R_{A}³, R_{A}⁴, and R_{A}⁵ are other than H;
R_{A}⁶ is H, OH, or halogen; and
A_{A} has the structure: 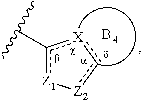
wherein
α, β, χ, and δ are each independently absent or present, and when present each is a bond;
X is C or N;
Z₁ is N;
Z₂ is N or NR_{A}⁹,
wherein R_{A}⁹ is H, C₁-C₄ alkyl, or oxetane;
B_{A} is a substituted or unsubstituted 5, 6, or 7 membered ring structure; or
a pharmaceutically acceptable salt thereof.

46. The method of claim 45, wherein the compound has the structure 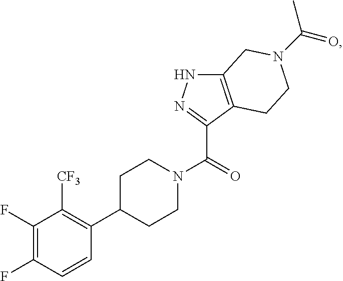 [SMILES: CC(=O)N1CCc2c(C(=O)N3CCC(c4ccc(F)c(F)c4C(F)(F)F)CC3)n[nH]c2C1]
or a pharmaceutically acceptable salt thereof.

47. The method of claim 45, wherein the compound has the structure 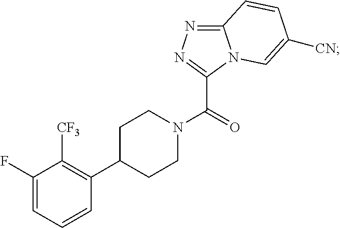 [SMILES: N#Cc1ccc2nnc(C(=O)N3CCC(c4cccc(F)c4C(F)(F)F)CC3)n2c1]
or a pharmaceutically acceptable salt thereof.

48. The method of claim 44, wherein the pharmaceutical composition comprises a compound having the structure of Formula (II): 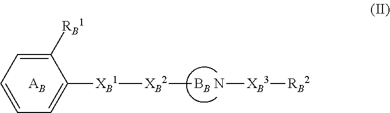
wherein:
ring A_{B} is benzene optionally further substituted;
R_{B}¹ is an optionally substituted branched C₃-C₆ alkyl group;
X_{B}¹ is O, S, SO, SO₂, or NH;
X_{B}² is a bond or a C₁-C₃ alkylene group;
ring B_{B} is azetidine or piperidine;
X_{B}³ is CO or SO₂;
R_{B}² is an optionally substituted hydrocarbon group, an optionally substituted heterocyclic group, an optionally substituted hydroxy group, an optionally substituted mercapto group, a cyano group, a nitro group, an acyl group, or a halogen atom; or
a pharmaceutically acceptable salt thereof.

49. The method of claim 48, wherein the compound is 4-(3-(2-tert-butylphenoxy)azetidin-1-yl)-4-oxobutanoic acid, 3-{3-[(2-tert-butyl-4-fluorophenoxy)methyl]azetidin-1-yl}-3-oxopropanoic acid, 2-{[3-(2-tert-butyl-4-chlorophenoxy)azetidin-1-yl]carbonyl}pyridine, 4-[3-(2-tert-butyl-4-chlorophenoxy)azetidin-1-yl]-4-oxobutanoic acid, {3-[(2-tert-butyl-4-chlorophenoxy)methyl]azetidin-1-yl}(oxo)acetic acid, {3-[(2-tert-butylphenoxy)methyl]azetidin-1-yl}(oxo)acetic acid, 3-{3-[(2-tert-butylphenoxy)methyl]azetidin-1-yl}-3-oxopropanoic acid, {4-[(2-tert-butyl-4-chlorophenoxy)methyl]piperidin-1-yl}(oxo)acetic acid, [4-(2-tert-butylphenoxy)piperidin-1-yl](oxo)acetic acid, or {4-[(2-tert-butylphenoxy)methyl]piperidin-1-yl}(oxo)acetic acid, or a pharmaceutically acceptable salt thereof.

50. The method of claim 44, wherein the pharmaceutical composition comprises a compound having the structure of Formula (III): 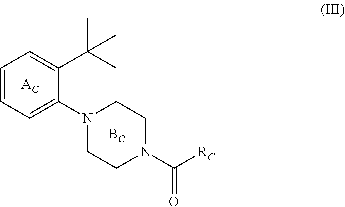
wherein:
ring A_{C} is a benzene ring optionally substituted by 1 to 3 substituents selected from the group consisting of (a) a halogen atom, and (b) a C₁-C₆ alkyl group;
ring B_{C} is a piperazine ring optionally substituted by 1 to 3 substituents selected from the group consisting of (a) a halogen atom, (b) a C₁-C₆ alkyl group optionally substituted by 1 to 3 halogen atoms, and (c) a C₁-C₆ alkoxy group optionally substituted by 1 to 3 halogen atoms; and
R_{C} is (1) an optionally substituted C₁-C₁₀ alkyl group, (2) an optionally substituted C₆-C₁₄ aryl group, (3) an optionally substituted 5- or 6-membered aromatic heterocyclic group, (4) an optionally substituted amino group, (5) an optionally substituted carboxy group, or (6) an optionally substituted carbamoyl group; or
a pharmaceutically acceptable salt thereof.

51. The method of claim 50, wherein the compound is N-{[4-(2-tert-butylphenyl)piperazin-1-yl]carbonyl}glycine, 3-[4-(2-tert-butylphenyl)piperazin-1-yl]-3-oxopropanoic acid, [4-(2-tert-butyl-4-chlorophenyl)piperazin-1-yl](oxo)acetic acid, 5-{2-[4-(2-tert-butylphenyl)piperazin-1-yl]-2-oxoethyl}imidazolidine-2,4-dione, [(5-{[4-(2-tert-butylphenyl)piperazin-1-yl]carbonyl}isoxazol-3-yl)oxy]acetic acid, or a pharmaceutically acceptable salt thereof.

52. The method of claim 44, wherein the pharmaceutical composition comprises a compound having the structure of Formula (IV): 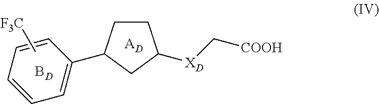
wherein:
ring A_{D} is a 5-membered non-aromatic heterocycle optionally further substituted by one oxo group;
ring B_{D} is a benzene ring optionally further substituted by 1 to 4 substituents; and
X_{D} is O, CH₂O, OCH₂, CH₂, (CH₂)₂, S, CH₂S, SCH₂, S(O), CH₂S(O), S(O)CH₂, S(O)₂, CH₂S(O)₂, or S(O)₂CH₂;
or a pharmaceutically acceptable salt thereof.

53. The method of claim 52, wherein the compound is ({(3S)-1-[3,5-bis(trifluoromethyl)phenyl]pyrrolidin-3-yl}oxy)acetic, ({1-[4-chloro-3-(trifluoromethyl)phenyl]pyrrolidin-3-yl}sulfanyl)acetic acid, 3-{(2R,5S)-5-[3,5-bis(trifluoromethyl)phenyl]tetrahydrofuran-2-yl}propanoic acid, or a pharmaceutically acceptable salt thereof.

54. The method of claim 44, wherein the pharmaceutical composition comprises a compound having the structure of Formula (V): 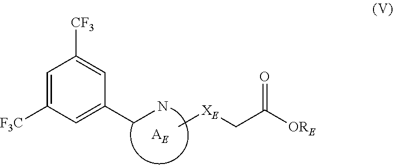
wherein;
ring A_{E} is a pyrazole ring, a pyridine ring, an oxazole ring, an imidazole ring, or a pyrimidine ring;
X_{E} is S, optionally substituted alkylene, or O; and
R_{E} is a hydrogen atom or a C₁-C₆ alkyl group; or
a pharmaceutically acceptable salt thereof.

55. The method of claim 54, wherein the compound is ((4-(3,5-bis(trifluoromethyl)phenyl)-1,3-oxazol-2-yl)sulfanyl)acetic acid, ethyl ((6-(3,5-bis(trifluoromethyl)phenyl)-pyridin-3-yl)sulfanyl)acetic acid, ((6-(3,5-bis(trifluoromethyl)-phenyl)pyridine-3-yl)sulfanyl)acetic acid, or 3-(3-(3,5-bis(trifluoromethyl)phenyl)-1H-pyrazol-1-yl)butanoic acid, or 3-{3-[3,5-bis(trifluoromethyl)phenyl]-1H-pyrazol-1-yl}propanoic acid (also known as STG-001).

56. The method of any one of claims 1-55, wherein the at least one of the at least two variables relates to one or more selected from the group comprising visual acuity, average score of functional visual questionnaire, and a measurement obtained from an imaging technology.

57. The method of any one of claims 1-55, wherein the at least one of the at least two variables relates to one or more selected from the group comprising a change in visual acuity, a change in average score of functional visual questionnaire, and a change in a measurement obtained from an imaging technology for detecting or quantifying a change in ocular structure or visual function.

58. The method of any one of claims 1-55, wherein the at least one of the at least two variables relates to a change in visual acuity, a change in average score of functional visual questionnaire, and a change in a measurement obtained from an imaging technology for detecting or quantifying a change in optic atrophy or visual function.

59. The method of any one of claims 1-55, wherein the at least two variables relate to a change in visual acuity, a change in average score of functional visual questionnaire, and a change in a measurement obtained from an imaging technology for detecting or quantifying a change in optic atrophy or visual function.

60. The method of any one of claims 1-55, wherein the at least two variables relate to a change in visual acuity and a change in average score of functional visual questionnaire.

61. The method of any one of claims 1-55, wherein the at least two variables relate to a change in visual acuity and a change in a measurement obtained from an imaging technology for detecting or quantifying a change in optic atrophy or visual function.

62. The method of any one of claims 1-55, wherein the at least two variables relate to a change in average score of functional visual questionnaire and a change in a measurement obtained from an imaging technology for detecting or quantifying a change in optic atrophy or visual function.

63. The method of any one of claims 1-55, wherein the at least two variables all relate to a change in visual acuity.

64. The method of any one of claims 1-55, wherein the at least two variables all relate to a change in average score of functional visual questionnaire.

65. The method of any one of claims 1-55, wherein the at least two variables all relate to a change in a measurement obtained from an imaging technology for detecting or quantifying a change in optic atrophy or visual function.

66. The method of any one of claims 1-55, wherein the at least one of the at least two variables relates a change in ocular structure, a change in ocular pathology, a change in retinal atrophy, a change in macular atrophy, a change in macular degeneration, or a combination thereof.

67. The method of any one of claims 1-66, wherein two of the at least two variables correlate with each other.

68. The method of any one of claims 1-66, wherein one of the at least two variables correlates with visual acuity.

69. The method of any one of claims 1-66, wherein one of the at least two variables correlates with a change in visual acuity.

70. The method of any one of claims 1-66, wherein all of the at least two variables correlate with a change in visual acuity.

71. The method of any one of claims 1-66, wherein one of the at least two variables correlates with a state of the ocular disease.

72. The method of any one of claims 1-66, wherein one of the at least two variables correlates with progression of the ocular disease.

73. The method of any one of claims 1-66, wherein the at least two variables comprise a value obtained from a logarithm of the minimum angle of resolution (log MAR) chart, a Snellen chart, a best corrected visual acuity (BCVA) test, an early treatment diabetic retinopathy study (ETDRS) letter test, or a combination thereof.

74. The method of any one of claims 1-66, wherein the at least two variables comprise a value obtained from a functional visual questionnaire best comprising one or more questions related to the following:
(a) the subject's confidence of moving to different places in the daytime or nighttime;
(b) the subject's ability to recognize people, to take part in sport or to get information; and
(c) the subject's ability for social interaction or work efficiency,
wherein each of the one or more questions is scored with respect to the subject.

75. The method of claim 74, wherein the each of the one or more questions is scored in a severity scale of 0-4 with respect to the subject according to the following:
(a) an answer of “NEVER” is scored 0;
(b) an answer of “ALMOST NEVER” is scored 1;
(c) an answer of “SOMETIMES” is scored 2;
(d) an answer of “ALMOST ALWAYS” is scored 3; and
(e) an answer of “ALWAYS” is scored 4.

76. The method of any one of claims 1-75, wherein the at least two variables comprise a value obtained from microperimetry (MP), perimetry, or both.

77. The method of claim 76, wherein the MP comprises measuring a spatial map of retinal sensitivity of the subject.

78. The method of claim 76, wherein the MP comprises measuring a mean retina sensitivity of the subject, or a change thereof.

79. The method of any one of claims 1-78, wherein the at least two variables comprise a value obtained from one or more selected from the group comprising fundus photography, fundus angiography (FA), fundus autofluorescence (FAF), spectral domain-optical coherence tomography (SD-OCT), quantitative Autofluorescence (qAF), infrared (IR) imaging, optical coherence tomography-angiography (OCT-A), and widefield imaging.

80. The method of any one of claims 1-79, wherein the at least two variables comprise one or more selected from the group comprising an area of questionable decreased autofluorescence (QDAF), an area of definite decreased autofluorescence (DDAF), an area of decreased autofluorescence (DAF), a retina thickness, an ellipsoid zone (EZ) defect width, a central subfield retina thickness (CST), an outer subfield retina thickness (OST), a middle subfield retina thickness (MST), and a change thereof.

81. The method of claim 80, wherein the area of decreased autofluorescence (DAF) comprises the area of questionable decreased autofluorescence (QDAF) and the area of definite decreased autofluorescence (DDAF).

82. The method of any one of claims 1-81, wherein the at least two variables comprise two or more selected from the group comprising visual acuity, QDAF, DAF, EZ defect width, or a change thereof.

83. The method of any one of claims 1-81, wherein the at least two variables comprise visual acuity, QDAF, DAF, EZ defect width, or a change thereof.

84. The method of claim 83, wherein the EZ defect width correlates with the visual acuity.

85. The method of claim 83, wherein the EZ defect width correlates with the QDAF.

86. The method of claim 83, wherein the EZ defect width correlates with the DAF.

87. The method of claim 83, wherein the visual acuity correlates with the QDAF.

88. The method of claim 83, wherein the EZ defect width correlates with the visual acuity, QDAF, and DAF.

89. The method of claim 83, wherein the visual acuity correlates with the EZ defect width and QDAF.

90. The method of claim 83, wherein the EZ defect width correlates with the visual acuity, QDAF, and DAF with a correlation coefficient of more than about 0.8 and a p value of less than about 0.05.

91. The method of claim 83, wherein the visual acuity correlates with the EZ defect width and QDAF with a p value of less than about 0.05.

92. The method of any one of claims 1-91, wherein the at least two variables of the subject are obtained from both the right and left eyes of the subject.

93. The method of any one of claims 1-92, wherein the method further comprises predicting whether prognostic potentials of treatment improve based on the one more composite biomarkers.

94. The method of any one of claims 1-93 wherein the treatment is therapeutically effective to treat the subject when the composite biomarker exceeds the threshold value.

95. The method of any one of claims 1-93 wherein the treatment is not therapeutically effective to treat the subject when the composite biomarker does not exceed the threshold value.
